(19) United States
(12) Patent Application Publication
(10) Pub. No.: US 2024/0212845 A1
(43) Pub. Date: Jun. 27, 2024

(54) ANIMAL DATA-BASED IDENTIFICATION AND RECOGNITION SYSTEM AND METHOD

(71) Applicant: SPORTS DATA LABS, INC., Royal Oak, MI (US)

(72) Inventors: Vivek KHARE, Cupertino, CA (US); Mark GORSKI, Royal Oak, MI (US); Stan MIMOTO, Bethel Island, CA (US)

(73) Assignee: SPORTS DATA LABS, INC., Royal Oak, MI (US)

(21) Appl. No.: 18/288,554

(22) PCT Filed: Apr. 27, 2022
(86) PCT No.: PCT/US2022/026532

§ 371 Date: Oct. 26, 2023

Related U.S. Application Data
(60) Provisional application No. 63/180,322, filed on Apr. 27, 2021.
(60) Provisional application No. 63/213,523, filed on Jun. 22, 2021.

(51) Int. Cl. G16H 50/20; G16H 10/60

(52) U.S. Cl. CPC G16H 50/20; G16H 10/60

(57) ABSTRACT

An animal data-based identification and recognition system includes one or more source sensors that gather animal data from an assumed or unknown subject wherein the animal data is transmitted electronically. One or more computing devices collect the animal data from the one or more source sensors. The one or more computing devices gather reference animal data related to a targeted subject, targeted medical condition, or targeted biological response. The one or more computing devices create, modify, or enhance at least one unique asset related to the targeted subject, the targeted medical condition, or the targeted biological response based upon the reference animal data. A comparison between the at least one created, modified, or enhanced unique asset and the animal data derived from the one or more source sensors, or its one or more derivatives, identifies the targeted subject, the targeted medical condition, or the targeted biological response.

### CROSS-REFERENCE TO RELATED APPLICATIONS

This application claims the benefit of U.S. provisional application Ser. No. 63/213,523 filed Jun. 22, 2021 and U.S. provisional application Ser. No. 63/180,322 filed Apr. 27, 2021, the disclosures of which are hereby incorporated in their entirety by reference herein.

### TECHNICAL FIELD

In at least one aspect, the present invention is related to systems and methods for identifying one or more animals, medical conditions, or biological responses based upon the combination of unique biological features of an animal-derived from sensor-based animal data.

### BACKGROUND

Animals have distinct biological features, with distinct biological-based patterns, rhythms, and characteristics for a variety of their biological functions, including heartbeats, ECG patterns, breathing rates, speech, and other processes that can identify animals, their medical conditions, and biological states. However, these unique patterns, rhythms, and characteristics can change based upon a number of factors, including activity, age, medical fitness, time, environmental conditions, and the like. This can make identification using biological-based data inaccurate and unreliable.

Accordingly, there is a need for a system that can create unique biological-based identifiers utilizing sensor-based animal data that can identify an animal, their one or more medical conditions, their one or more biological responses, or a combination thereof, from other animals, other medical conditions, and other biological responses.

### SUMMARY

In at least one aspect, an animal data-based identification and recognition system is described. The system includes one or more source sensors that gather animal data from an assumed or unknown subject (i.e., targeted subject), wherein the animal data is transmitted electronically. One or more computing devices collect the animal data from the one or more source sensors. The one or more computing devices gather reference animal data related to a targeted subject, targeted medical condition, or targeted biological response. In a variation, the one or more computing devices gather reference animal data related to a plurality of targeted subjects, targeted medical conditions, or targeted biological responses. In another variation, the one or more computing devices gather reference animal data related to a targeted subject and one or more targeted medical conditions, one or more targeted biological responses, or a combination thereof. The one or more computing devices create, modify, or enhance at least one unique asset related to the targeted subject, the targeted medical condition, or the targeted biological response based upon the reference animal data. The one or more computing devices evaluate (e.g., compare, analyze) the at least one created, modified, or enhanced unique asset with at least a portion of the animal data derived from the one or more source sensors, or its one or more derivatives, from the assumed or unknown subject. The evaluation (e.g., comparison, analysis) between the at least one created, modified, or enhanced unique asset and the animal data derived from the one or more source sensors, or its one or more derivatives, identifies the assumed or unknown subject as the targeted subject, identifies the assumed or unknown subject as having the targeted medical condition, identifies the assumed or unknown subject as having (i.e., exhibiting) the targeted biological response, or a combination thereof. In variations related to identifying one or more targeted medical conditions or targeted biological responses, the subject may be known.

In another aspect, an animal data-based identification and recognition system is described. The system includes a collecting computing device that gathers animal data derived from one or more source sensors from an assumed, known, or unknown subject (i.e., a targeted subject). The animal data is transmitted electronically. The collecting computing device gathers reference animal data related to a targeted subject (e.g., which can include gathering reference animal data from multiple subjects), one or more medical conditions, or one or more biological responses, the collecting computing device being operable to derive at least one unique asset from the reference animal data and related to the targeted subject, the one or more medical conditions, or the one or more biological responses. The collecting computing device compares the at least one unique asset derived from the reference animal data with at least a portion of the gathered animal data derived from the one or more source sensors, or the animal data's one or more derivatives (e.g., another unique asset). The comparison between the at least one unique asset and the gathered animal data derived from the one or more source sensors, or its one or more derivatives, identifies the assumed, known, or unknown subject as the targeted subject, identifies the assumed, known, or unknown subject as having the one or more medical conditions, identifies the assumed, known, or unknown subject as having (i.e., exhibiting) the one or more biological responses, or a combination thereof.

In another aspect, an animal data-based identification and recognition system is described. The system includes one or more computing devices that gather reference animal data derived from, at least in part, one or more sensors wherein the one or more computing devices are operable to create, modify, or enhance at least one unique asset from the reference animal data for one or more known subjects that identify each of the one or more known subjects. One or more source sensors gather animal data from a targeted subject wherein the animal data is transmitted electronically. A collecting computing device (1) gathers the animal data from the targeted subject via the one or more source sensors, (2) creates, modifies, or enhances at least one unique asset from at least a portion of the animal data derived from the targeted subject via the one or more source sensors for the purpose of identifying the targeted subject as a known subject, and either (i) gathers the at least one created, modified, or enhanced unique asset derived from the reference animal data for the one or more known subjects, or (ii) provides the at least one unique asset derived from the targeted subject via the one or more source sensors, at least in part, to the one or more computing devices. The collecting computing device or the one or more computing devices evaluate (e.g., compare) the at least one created, modified, or enhanced unique asset from the one or more known subjects with the at least one created, modified, or enhanced unique asset from the targeted subject. The evaluation (e.g., comparison) between the two or more unique assets enables the collecting computing device or the one or more computing devices to identify the targeted subject as a known subject. Furthermore, the identification of the targeted subject as a known subject enables the system to verify the association between one or more source sensors, the gathered animal data, and the targeted subject.

In another aspect, an animal data-based identification and recognition system is described. The system includes one or more computing devices that gather reference animal data derived from, at least in part, one or more sensors wherein the one or more computing devices are operable to create, modify, or enhance at least one unique asset for one or more known medical conditions or biological responses from the reference animal data that identify each of the one or more known medical conditions or biological responses. One or more source sensors gather animal data from a targeted subject wherein the animal data is transmitted electronically. A collecting computing device (1) gathers the animal data from the targeted subject via the one or more source sensors, (2) creates, modifies, or enhances at least one unique asset from at least a portion of the animal data derived from the one or more source sensors to identify one or more medical conditions or biological responses associated with (e.g., related to, derived from, of) the targeted subject, and either (i) gathers the at least one created, modified, or enhanced unique asset derived from the reference animal data for the one or more known medical conditions or known biological responses, or (ii) provides the at least one unique asset derived from the targeted subject via the one or more source sensors, at least in part, to the one or more computing devices. The collecting computing device or the one or more computing devices evaluate (e.g., compare) the at least one created, modified, or enhanced unique asset for the one or more known medical conditions or biological responses with the at least one unique asset from the targeted subject. The evaluation (e.g., comparison) between the two or more unique assets enables the collecting computing device or the one or more computing devices to identify one or more of the known medical conditions or biological responses associated with the targeted subject.

In another aspect, a sensor authentication and verification system related to animal data is described. The system is designed to verify the association between a targeted subject and at least one source sensor. The system includes two or more sensors, at least one of which is a primary source sensor and at least one of which is a secondary sensor. Each of the two or more sensors is operable to receive one or more signals (e.g., receive instructions to perform an action) from a collecting computing device. Characteristically, the one or more signals are transmitted electronically. The at least one primary source sensor is operable to gather animal data from a targeted subject and provide at least a portion of the animal data to the collecting computing device. The secondary sensor is operable to provide information related to the targeted subject, the at least one primary source sensor, or a combination thereof, to the collecting computing device. The collecting computing device sends one or more signals to the at least one primary source sensor to take one or more actions. The one or more actions taken by the at least one primary source sensor are captured (e.g., identified, observed) by the one or more secondary sensors, enabling the one or more secondary sensors to provide information to the collecting computing device related to the one or more actions, the targeted subject, or a combination thereof, to identify the at least one primary source sensor. The collecting computing device identifies the at least one primary source sensor. The collecting computing device is further operable to (1) authenticate the identity of the targeted subject associated with the at least one primary source sensor via at least a portion of animal data gathered from the one or more secondary sensors, at least a portion of animal data gathered from the at least one primary source sensor, or a combination thereof, and (2) verify the association between the at least one primary source sensor and the targeted subject. Upon verification, the system is operable to assign the at least one primary source sensor to the targeted subject.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements and wherein:

FIG. 1 provides a schematic illustration of a system that enables the identification of one or more animals via their sensor-based animal data, as well as the identification of one or more medical conditions or biological responses related to one or more animals via their sensor-based animal data.

FIG. 2 provides a schematic illustration of a system that enables authentication and verification of one or more sensors associated with one or more animals.

### DETAILED DESCRIPTION

Reference will now be made in detail to presently preferred compositions, embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

It is also to be understood that this invention is not limited to the specific embodiments and methods described herein, as specific components, parameters, and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The phrase “data is” is meant to include both “datum is” and “data are,” as well as all other possible meanings, and is not intended to be limiting in any way.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

The term “one or more” means “at least one” and the term “at least one” means “one or more.” The terms “one or more” and “at least one” include “plurality” and “multiple” as a subset. In a refinement, “one or more” includes “two or more.”

The term “or its one or more derivatives” can be interchangeable with “and its one or more derivatives” depending on the use case and is not intended to be limiting in any way.

With respect to the terms “bet” and “wager,” both terms mean an act of taking a risk (e.g., which can be monetary or non-monetary in nature) on the outcome of a future event. Risk includes both financial (e.g., monetary) and non-financial risk (e.g., health, life). A risk can be taken against another one or more parties (e.g., an insurance company deciding whether to provide insurance; a security system deciding whether to provide access to information to, or authenticate, another individual; a healthcare system deciding whether to administer one drug versus another drug, or one treatment plan versus another treatment plan, to an individual in a healthcare setting, and the like) or against oneself (e.g., an individual deciding whether to obtain insurance), on the basis of an outcome, or the likelihood of an outcome, of a future event. Examples include gambling (e.g., sports betting), insurance, security, healthcare, and the like. Where one of these two terms are used herein, the presently disclosed and claimed subject matter can use either of the other two terms interchangeably.

Throughout this application, where publications are referenced, the disclosures of these publications in their entireties are hereby incorporated by reference into this application to more fully describe the state of the art to which this invention pertains.

The term “server” refers to any computer or computing device (including, but not limited to, desktop computer, notebook computer, laptop computer, mainframe, mobile phone, smart watch, hearables, smart contact lens, head-mountable units such as smart-glasses, headsets such as augmented reality headsets, virtual reality headsets, mixed reality headsets, and the like, augmented reality devices, virtual reality devices, mixed reality devices, and the like), distributed system, blade, gateway, switch, processing device, or a combination thereof adapted to perform the methods and functions set forth herein.

The term “computing device” refers generally to any device that can perform at least one function, including communicating with another computing device. In a refinement, a computing device includes a central processing unit that can execute program steps and memory for storing data and a program code.

When a computing device is described as performing an action or method step, it is understood that the one or more computing devices are operable to and/or configured to perform the action or method step typically by executing one or more lines of source code. The actions or method steps can be encoded onto non-transitory memory (e.g., hard drives, optical drive, flash drives, and the like).

The term “derivative” wherein referring to data means that the data is mathematically transformed to produce the derivative as an output. In a refinement, a mathematic function receives the data as input and outputs the derivative as an output.

The term “electronic communication” means that an electrical signal is either directly or indirectly sent from an originating electronic device to a receiving electronic device. Indirect electronic communication can involve processing of the electrical signal, including but not limited to, filtering of the signal, amplification of the signal, rectification of the signal, modulation of the signal, attenuation of the signal, adding of the signal with another signal, subtracting the signal from another signal, subtracting another signal from the signal, and the like. Electronic communication can be accomplished with wired components, wirelessly-connected components, or a combination thereof.

The processes, methods, or algorithms disclosed herein can be deliverable to or implemented by a computer, controller, or other computing device, which can include any existing programmable electronic control unit or dedicated electronic control unit. Similarly, the processes, methods, or algorithms can be stored as data and instructions executable by a computer, controller, or other computing device in many forms including, but not limited to, information permanently stored on non-writable storage media such as ROM devices and information alterably stored on writeable storage media such as floppy disks, magnetic tapes, CDs, RAM devices, other magnetic and optical media, and shared or dedicated cloud computing resources. The processes, methods, or algorithms can also be implemented in an executable software object. Alternatively, the processes, methods, or algorithms can be embodied in whole or in part using suitable hardware components, such as Application Specific Integrated Circuits (ASICs), Field-Programmable Gate Arrays (FPGAs), state machines, controllers or other hardware components or devices, or a combination of hardware, software, and firmware components.

The terms “subject” and “individual” are synonymous, interchangeable, and refer to a human or other animal, including birds, reptiles, amphibians, and fish, as well as all mammals including, but not limited to, primates (particularly higher primates), horses, sheep, dogs, rodents, pigs, cats, rabbits, and cows. The one or more subjects or individuals may be, for example, humans participating in athletic training or competition, horses racing on a race track, humans playing a video game, humans monitoring their personal health, humans providing their animal data to a third party (e.g., insurance system, health system, monetization system), humans participating in a research or clinical study, humans participating in a fitness class, and the like. A subject or individual can also be a derivative of a human or other animal (e.g., lab-generated organism derived at least in part from a human or other animal), one or more individual components, elements, or processes of a human or other animal (e.g., cells, proteins, biological fluids, amino acid sequences, tissues, hairs, limbs) that make up the human or other animal, one or more digital representations that share at least one characteristic with a human or other animal (e.g., data set representing a human that shares at least one characteristic with a human representation in digital form—such as sex, age, biological function as examples—but is not generated from any human that exists in the physical world; a simulated individual or digital individual that is based on, at least in part, a real-world human or other animal, such as a digital representation of an individual or avatar in a virtual environment or simulation such as a video game or metaverse), or one or more artificial creations that share one or more characteristics with a human or other animal (e.g., lab-grown human brain cells that produce an electrical signal similar to that of human brain cells). In a refinement, the subject or individual can be one or more programmable computing devices such as a machine (e.g., robot, autonomous vehicle, mechanical arm) or network of machines that share at least one biological function with a human or other animal and from which one or more types of biological data can be derived, which may be, at least in part, artificial in nature (e.g., data from artificial intelligence-derived activity that mimics biological brain activity; biomechanical movement data derived a programmable machine that mimics biomechanical movement of an animal).

The term “animal data” refers to any data obtainable from, or generated directly or indirectly by, a subject that can be transformed into a form that can be transmitted to a server or other computing device. Typically, the animal data is electronically transmitted via a wired or wireless connection. Animal data includes, but is not limited to, any subject-derived data, including any signals or readings, that can be obtained from one or more sensors or sensing equipment/systems, and in particular, biological sensors (biosensors), as well as its one or more derivatives. Animal data also includes any biological phenomena capable of being captured from a subject and converted to electrical signals that can be captured by one or more sensors, descriptive data related to a subject (e.g., name, age, height, eye color, gender, anatomical information), auditory data related to a subject, visually-captured data related to a subject (e.g., image, likeness, observable information related to the subject), neurologically-generated data (e.g., brain signals from neurons), evaluative data related to a subject (e.g., skills of a subject), data that can be manually entered or gathered related to a subject (e.g., medical history, social habits, feelings of a subject, mental health data, financial information, subjective data), and the like (e.g., attributes/characteristics of the individual). The term “animal data” can be meant to include one or more types of animal data. In a refinement, the term “animal data” is inclusive of any derivative of animal data, including one or more computed assets, unique assets, insights, predictive indicators, artificial data (e.g., simulated animal data in a virtual environment, video game, or other simulation derived from the digital representation of the subject), or a combination thereof. In another refinement, animal data includes one or more inputs (e.g., signals, readings, other data) from one or more non-animal data sources. In another refinement, animal data includes at least a portion of non-animal data that provides contextual information related to the animal data. In another refinement, animal data includes any metadata gathered or associated with the animal data. In another refinement, animal data includes at least a portion of simulated data. In another refinement, animal data is inclusive of simulated data.

The term “reference animal data” refers to any animal data used as a reference to classify, categorize, or evaluate (e.g., compare, analyze) other animal data, as well as to derive information from other data. It can include any available, accessible, or gathered data, including any type of animal data and/or non-animal data either directly or indirectly related to (or derived from) one or more targeted subjects, medical conditions, or biological responses that enable the identification (e.g., including partial identification, non-identification) of the one or more targeted subjects, medical conditions, or biological responses. It can also include any previously-collected animal data (e.g., historical animal data), including previously-collected animal data derived from one or more sensors as well as previously collected (e.g., historical) non-animal data either directly or indirectly related to (or associated with) the previously-collected animal data. Reference animal data can be gathered from any number of subjects (e.g., tens, hundreds, thousands, millions, billions, and the like) and data sources (e.g., it can be gathered from sensors or computing devices, manually inputted, artificially created, derived from one or more actions, and the like). It can be structured (e.g., created, curated) in a way to facilitate one or more evaluations (e.g., comparisons) of (or between) data sets and/or derivatives of data sets (e.g., unique assets). In a refinement, reference animal data includes at least a portion of non-animal data (e.g., including non-animal contextual data to provide additional context to the animal data). In another refinement, reference animal data includes at least a portion of simulated animal data (e.g., the system may generate artificial animal data as reference animal data; the system may run one or more simulations, the output of which can be reference animal data; one or more animal data sets may include simulated data; and the like). In another refinement, reference animal data includes metadata gathered or associated with animal data. In another refinement, reference animal data includes any animal data derived either directly or indirectly from any subject, with the animal data being structured in a way to facilitate one or more evaluations (e.g., comparisons) of data sets (e.g., including any derivatives) to enable identification of one or more targeted subjects, medical conditions, or biological responses. In a variation, reference animal data includes data that is not derived directly or indirectly from the targeted individual (e.g., data from another one or more individuals) but shares at least one attribute (e.g., characteristic) with the one or more targeted individuals, medical conditions, or biological responses. In another refinement, reference animal data is inclusive of any derivative of animal data, including one or more signals, readings, computed assets, unique assets, insights, predictive indicators, or artificial data. In another refinement, reference animal data can include identifiable, de-identified (e.g., pseudonymized), semi-anonymous, or anonymous data tagged with metadata (e.g., that has associated metadata) related to one or more biological responses or medical conditions. In another refinement, reference animal data includes data derived from the one or more biological responses and/or medical conditions derived from identifiable, anonymized, semi-anonymized, or de-identified (e.g., pseudonymized) sources. In another refinement, reference animal data can be categorized or grouped together to form one or more units of such data. In another refinement, reference animal data can be dynamically created, modified, or enhanced with one or more additions, changes, or removal of non-functioning data (e.g., data that the system will remove or stop using). In another refinement, at least a portion of the reference animal data may be weighted based upon one or more characteristics of (or related to) the one or more sensors (e.g., reference animal data from sensors that produce average quality data may have a lower weighted score than reference animal data from sensors that produce high-quality data), the one or more individuals or groups of individuals, the animal data, the non-animal data associated with the animal data, or a combination thereof. In another refinement, the system may be operable to conduct one or more data audits on reference animal data. For example, the system may recall reference animal data originating from one or more sensors based upon one or more sensor characteristics (e.g., a faulty data gathering functionality within the one or more sensors could cause the system to recall and remove the data from the reference animal data database to enable more accurate identification). In another refinement, the reference data includes previously collected animal data that are typically analyzed and characterized. In a further refinement, at least a portion of previously collected animal data is derived from one or more sensors.

The term “artificial data” refers to artificially-created data that is derived from, based on, or generated using, at least in part, animal data or one or more derivatives thereof. It can be created by running one or more simulations utilizing one or more artificial intelligence techniques or statistical models and can include one or more inputs (e.g., signals, readings, other data) from one or more non-animal data sources. In a refinement, artificial data includes any artificially-created data that shares at least one biological function with a human or another animal (e.g., artificially-created vision data, artificially-created movement data). The term “artificial data” is inclusive of “synthetic data,” which can be any production data applicable to a given situation that is not obtained by direct measurement. Synthetic data can be created by statistically modeling original data and then using the one or more models to generate new data values that reproduce at least one of the original data's statistical properties. In another refinement, the term “artificial data” is inclusive of any derivative of artificial data. In another refinement, artificial data is generated utilizing at least a portion of reference animal data. For the purposes of the presently disclosed and claimed subject matter, the terms “simulated data” and “synthetic data” are synonymous and used interchangeably with “artificial data” (and vice versa), and a reference to any one of the terms should not be interpreted as limiting but rather as encompassing all possible meanings of all the terms. In another refinement, the term “artificial data” is inclusive of the term “artificial animal data.”

The term “insight” refers to one or more descriptions or indicators that can be assigned to a targeted individual that describe a condition or status of, or related to, the targeted individual utilizing at least a portion of their animal data. Examples include descriptions or other characterizations related to an individual's stress levels (e.g., high stress, low stress), energy or fatigue levels, bodily responses, medical conditions, and the like. An insight may be quantified by one or more numbers (e.g., including a plurality of one or more numbers) and may be represented as a probability or similar odds-based indicator. An insight may also be quantified, communicated, or characterized by one or other metrics or indices of animal data-based performance that are predetermined (e.g., codes, graphs, charts, plots, colors or other visual representations, plots, readings, numerical representations, descriptions, text, physical responses such as a vibration, auditory responses, visual responses, kinesthetic responses, or verbal descriptions). An insight may also include one or more visual representations related to a condition or status of one or more targeted subjects (e.g., an avatar or virtual depiction of a targeted subject visualizing future weight loss goals on the avatar or depiction of the targeted subject). In a refinement, an insight is a personal score or other indicator related to one or more targeted individuals or groups of targeted individuals (e.g., including their one or more medical conditions and/or biological responses) that utilizes at least a portion of animal data to (1) evaluate, assess, prevent, or mitigate animal data-based risk; (2) evaluate, assess, or optimize animal data-based performance (e.g. biological performance); or a combination thereof. The personal score or other indicator can be utilized by the one or more targeted subjects from which the animal data or one or more derivatives thereof are derived from, as well as one or more third parties (e.g., insurance organizations, healthcare providers or professionals, sports performance coaches, medical billing organizations, fitness trainers, employers, virtual environment operators, sports betting companies, data monetization companies, and the like). In another refinement, an insight is derived from one or more computed assets. In another refinement, an insight is derived from one or more predictive indicators. In another refinement, an insight is derived from two or more types of animal data. In another refinement, an insight is derived related to a targeted subject or group of targeted subjects using at least a portion of animal data not derived from the targeted subject or group of targeted subjects. In another refinement, an insight includes one or more inputs (e.g., signals, readings, other data) from one or more non-animal data sources in one or more computations, calculations, measurements, derivations, incorporations, simulations, extractions, extrapolations, modifications, enhancements, creations, combinations, estimations, deductions, inferences, determinations, processes, communications, and the like. In another refinement, a unique asset includes information derived from one or more insights, and vice versa. In another refinement, an insight includes a plurality of insights. In another refinement, an insight is derived utilizing at least a portion of reference animal data. In another refinement, an insight is assigned to multiple targeted individuals. In yet another refinement, an insight is assigned to one or more groups of targeted individuals.

The term “computed asset” refers to one or more numbers, a plurality of numbers, values, metrics, readings, insights, graphs, charts, or plots that are derived from at least a portion of the animal data or one or more derivatives thereof (e.g., which can be inclusive of simulated data). For example, in the context of sensor-derived animal data, the one or more sensors used herein initially provide an electronic signal. The computed asset is extracted or derived, at least in part, from the one or more electronic signals or one or more derivatives thereof. The computed asset can describe or quantify an interpretable property of the one or more targeted individuals or groups of targeted individuals. For example, a computed asset such as electrocardiogram readings can be derived from analog front end signals (e.g., the electronic signal from the sensor), heart rate data (e.g., heart rate beats per minute) can be derived from an electrocardiogram or PPG sensors, body temperature data can be derived from temperature sensors, perspiration data can be derived or extracted from perspiration sensors, glucose information can be derived from biological fluid sensors, DNA and RNA sequencing information can be derived from sensors that obtain genomic and genetic data, brain activity data can be derived from neurological sensors, hydration data can be derived from in-mouth saliva or sweat analysis sensors, location data can be derived from GPS/optical/RFID-based sensors, biomechanical data can be derived from optical or translation sensors, and breathing rate data can be derived from respiration sensors. In a refinement, a computed asset includes one or more inputs (e.g., signals, readings, other data) from one or more non-animal data sources in one or more computations, calculations, measurements, derivations, incorporations, simulations, extractions, extrapolations, modifications, enhancements, creations, combinations, estimations, deductions, inferences, determinations, processes, communications, and the like. In another refinement, a unique asset includes information derived from one or more computed assets, and vice versa. In another refinement, a computed asset is derived from two or more types of animal data. In another refinement, a computed asset includes a plurality of computed assets. In another refinement, a computed asset may be derived utilizing at least a portion of simulated data.

The term “unique asset” refers to one or more biological-based signatures (e.g., unique digital signatures; in some variations, non-unique digital signatures), identifiers (e.g., unique identifiers; in some variations, non-unique identifiers), patterns (e.g., any type of pattern including time slice, spatial, spatiotemporal, temporospatial, and the like), rhythms, trends, features, measurements, outliers, abnormalities, anomalies, readings, signals, data sets, characteristics/attributes (e.g., unique characteristics), or a combination thereof, derived from one or more calculations, computations, measurements, derivations, extractions, extrapolations, simulations, creations, combinations, modifications, enhancements, estimations, evaluations, inferences, establishments, determinations, conversions, deductions, or observations from (or of) animal data, at least in part, that enable the identification of one or more targeted individuals, medical conditions, or biological responses. Characteristically, the at least one unique asset includes at least a portion of animal data. In many variations, the at least one unique asset enables identification of an individual, medical condition, or biological response based upon their one or more biological processes (e.g., the one or more unique biological signals, systems, processes, and the like that comprise the bodily functions of the individual), one or more characteristics/attributes of—or associated with—the individual, or a combination thereof. In these variations, the identification of the one or more biological processes are derived from at least a portion of the individual's animal data gathered, at least in part, via one or more sensors. In a refinement, the at least one unique asset uses animal data derived from two or more source sensors to create, modify, or enhance the at least one unique asset. In another refinement, the at least one unique asset uses two or more types of animal data to create, modify, or enhance the at least one unique asset. In another refinement, the at least one unique asset uses two or more types of animal data derived from the same sensor (e.g., source sensor) to create, modify, or enhance the at least one unique asset. In another refinement, the at least one unique asset uses two or more types of animal data derived from two or more sensors (e.g., source sensors) to create, modify, or enhance the at least one unique asset. In a further refinement, the at least one unique asset uses two or more types of animal data derived from the same source sensor to create, modify, or enhance the at least one unique asset. In another refinement, the at least one unique asset can be applied as an identification asset for multiple targeted subjects, medical conditions, or biological responses. In another refinement, the at least one unique asset includes at least a portion of non-animal data. In another refinement, the at least one unique asset is derived from reference animal data. In another refinement, the at least one unique asset is derived from simulated data. In another refinement, the creation, modification, or enhancement of the at least one unique asset occurs utilizing at least a portion of animal data, artificial data, reference animal data, non-animal data, or a combination thereof. In another refinement, the at least one unique asset enables the verification and/or classification (e.g., categorization) of one or more targeted individuals, medical conditions, or biological responses. In another refinement, the at least one unique asset enables authentication of one or more sensors (e.g., authenticating that the one or more sensors are, in fact, being used to collect animal data from the targeted subject) and the associated animal data (e.g., to ensure the animal data is associated with the correct targeted subject). In another refinement, the at least one unique asset is created, modified, or enhanced from two or more types of animal data that are captured across one or more time periods and one or more activities. For example, a unique asset such as a unique biological signature may be created for an individual based upon information derived from multiple computed assets or insights, captured across multiple time periods and multiple activities. In another refinement, the at least one unique asset is created, modified, or enhanced using two or more types of animal data, collected across two or more time periods, collected when the targeted subject is engaged in one or more activities, or a combination thereof. In another refinement, the at least one unique asset can be unique to a targeted individual, medical condition, or biological response, or a subset of targeted individuals, medical conditions, and biological responses. In another refinement, the at least one unique asset is not unique to a specific targeted individual, medical condition, or biological response, but rather can be applied to multiple targeted individuals, medical conditions, or biological responses. In another refinement, the at the least one unique asset is applicable to two or more identifications amongst one or more individuals, medical conditions, biological responses, or a combination thereof (e.g., a single unique asset may identify the individual and a biological response, or a medical condition and biological response, or the targeted individual and a medical condition, and the like). In another refinement, the at least one unique asset is applicable to two or more identifications, at least one of which is the targeted individual. In another refinement, the at least one unique asset is created, modified, or enhanced using one or more artificial intelligence techniques. In another refinement, the at least one unique asset is created, modified, or enhanced using one or more artificial intelligence techniques that produce one or more biological representations of the targeted individual (e.g., interpretable information related to the targeted individual's biological responses—derived from their animal data—in a variety of contexts) for the purposes of understanding one or more biological functions or processes of the targeted individual based upon their animal data to create, modify, or enhance the at least one unique asset. In another refinement, a unique asset includes of a plurality of unique assets.

The term “predictive indicator” refers to a metric or other indicator (e.g., one or more colors, codes, numbers, values, graphs, charts, plots, readings, numerical representations, descriptions, text, physical responses, auditory responses, visual responses, kinesthetic responses) derived either directly or indirectly from the comparison of (i) two or more unique assets, or (ii) at least one unique asset derived from reference animal data and gathered animal data derived from the one or more sensors (or its one or more derivatives), from which one or more forecasts, predictions, probabilities, assessments, possibilities, projections, determinations or recommendations related to one or more events (e.g., current events, outcomes for one or more future events such as health or medical events) that includes one or more targeted individuals, or one or more groups of targeted individuals, can be calculated, computed, derived, extracted, extrapolated, quantified, simulated, created, modified, assigned, enhanced, estimated, evaluated, inferred, established, converted, deduced, observed, communicated, or actioned upon. In a refinement, a predictive indicator is a calculated computed asset. In another refinement, a predictive indicator includes one or more inputs (e.g., signals, readings, other data) from one or more non-animal data sources as one or more inputs in the one or more calculations, computations, measurements, derivations, extractions, extrapolations, simulations, creations, combinations, modifications, assignments, enhancements, estimations, evaluations, inferences, establishments, determinations, conversions, deductions, observations, or communications of its one or more forecasts, predictions, probabilities, possibilities, assessments, projections, or recommendations. In another refinement, a predictive indicator includes at least a portion of simulated data as one or more inputs in the one or more calculations, computations, measurements, derivations, extractions, extrapolations, simulations, creations, combinations, modifications, assignments, enhancements, estimations, evaluations, inferences, establishments, determinations, conversions, deductions, observations, or communications of its one or more forecasts, predictions, probabilities, possibilities, assessments, projections, or recommendations. In another refinement, a unique asset includes information derived from one or more predictive indicators, and vice versa. In another refinement, a predictive indicator is derived utilizing at least a portion of reference animal data. In another refinement, a predictive indicator is derived from two or more types of animal data. In yet another refinement, a predictive indicator includes a plurality of predictive indicators.

For the purposes of this invention, any reference to the collection or gathering of animal data from one or more sensors from a subject includes gathering the animal data from one or more computing devices associated with the one or more sensors (e.g., a cloud or other computing device associated with the one or more sensors where the data is stored or accessible). Additionally, the terms “gathering” and “collecting” can be used interchangeably, and reference to any one of the terms should not be interpreted as limiting but rather as encompassing all possible meanings of both terms. In a refinement, the terms “gathering” and “collecting” can be used interchangeably with the term “receiving” (and vice versa), and reference to any one of the terms should not be interpreted as limiting but rather as encompassing all possible meanings of all the terms.

The term “modify” can be inclusive of “revise,” “amend,” “update,” “adjust,” “change,” and “refine.” Additionally, the term “create” can be inclusive of “derive” and vice versa. Similarly, “create” can be inclusive of “generate” and vice versa. In a refinement, “create” can also include an action that is calculated, computed, derived, extracted, extrapolated, simulated, combined, modified, enhanced, estimated, evaluated, inferred, established, determined, converted, or deduced. The term “enhance” refers to an improvement of quality or value in data and in particular the animal data or its one or more derivatives (e.g., unique asset, predictive indicator, insight).

A modification or enhancement of data can occur (1) as new data (e.g., animal data, non-animal data) is gathered by the system; (2) based upon one or more evaluations of existing data (e.g., one or more new signatures, identifiers, patterns, rhythms, trends, features, measurements, outliers, abnormalities, anomalies, readings, signals, data sets, characteristics/attributes, or a combination thereof are identified in existing data sets by the system); (3) as existing data is removed or replaced in the system; (4) as the system learns one or more new methods of transforming existing data into new data sets or deriving new data sets from existing data (e.g., the system learns to derive respiration rate data from raw sensor data that is traditionally used to extrapolate ECG data); (5) as new data is generated artificially; (6) as a result of one or more simulations; and the like. For example, new data entering the system may enhance the distinctiveness of the unique asset for a targeted individual or increase the ways in which a unique asset can be created. In another example, new data entering the system may enhance the accuracy of the system's predictive indicator. In another example, a data set or animal data derivative may be modified if data is removed from, or replaced in, the system (e.g., the system's removal of data from the reference animal data database may enable a more accurate identification of a targeted individual). In some variations, modification may result in a decrease in quality or value of the animal data or its one or more derivatives (e.g., decrease in identification accuracy of the unique asset or prediction accuracy).

The term “or a combination thereof” can mean any subset of possibilities or all possibilities. In a refinement, “or a combination thereof” includes both “or combinations thereof” and “and combinations thereof.”

The term “neural network” refers to a machine learning model that can be trained with training input to approximate unknown functions. In a refinement, neural networks include a model of interconnected digital neurons that communicate and learn to approximate complex functions and generate outputs based on a plurality of inputs provided to the model.

In a refinement, one or more comparisons or a step of comparing occur when the system utilizes one or more programs, which may incorporate one or more techniques (e.g., machine learning techniques, deep learning techniques, or statistical techniques), to measure, observe, calculate, derive, extract, extrapolate, simulate, create, combine, modify, enhance, estimate, evaluate, infer, establish, determine, convert, or deduce one or more similarities, dissimilarities, or a combination thereof, between two or more animal data sets (e.g., which can include one or more derivatives of animal data and its associated metadata), at least one of which is derived from reference animal data and at least one of which is derived—at least in part—from one or more source sensors, the two or more animal data sets each incorporating one or more biological-based signatures, identifiers, patterns, rhythms, trends, features, measurements, outliers, abnormalities, anomalies, readings, or characteristics/attributes, or a combination thereof that enable the identification of one or more targeted individuals, medical conditions, or biological responses. In one scenario, a comparison occurs when the system utilizes a sophisticated ensemble clustering algorithm that uses a combination of clustering algorithms that can include Density-Based Spatial Clustering Of Applications With Noise (DBSCAN), BIRCH, Gaussian Mixture Model (GMM), Hierarchical Clustering Algorithm (HCA) and Spectral-based clustering while using metrics of similarity grouping that can include inertia and silhouette scoring, as well as information criteria scores to identify the group or cluster. The output of the above methodology map gives data to a cluster or group. Within the identified group, one or more additional machine learning algorithms can be used that measure the nearness of data to similar sub-groups to identify, at least in part, the potential target the given data belongs to.

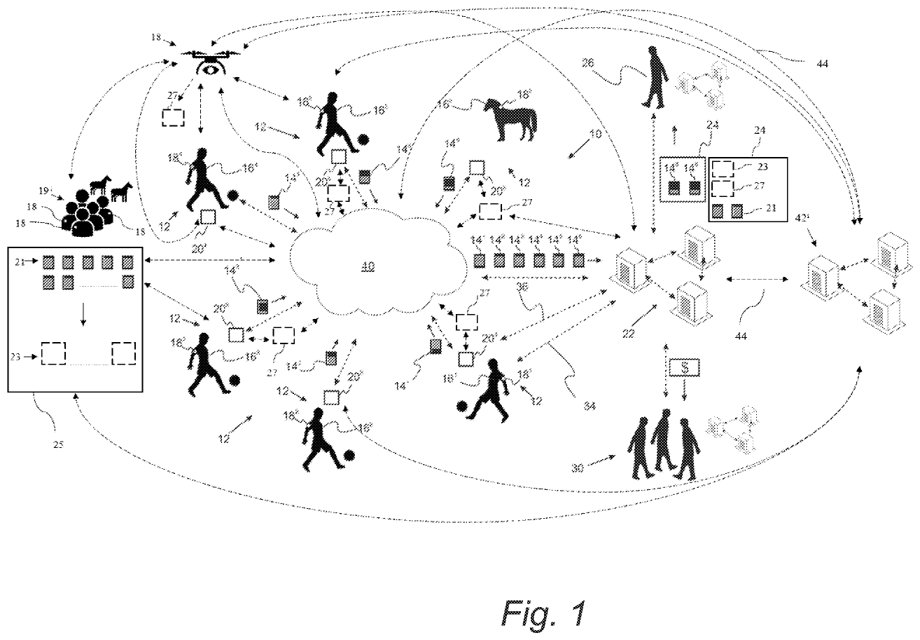

With reference to FIG. 1, a schematic of a system for an animal data-based identification and recognition system is provided. Animal data-based identification and recognition system 10 includes one or more sources 12 of animal data 14^{k} that can be transmitted electronically. Label k is merely an integer label from 1 to kₘₐₓ associated with each instance of the animal data where kₘₐₓ is the total number of instances of animal data. In this context, transmitted electronically includes being provided in an electronic form (e.g., digital form). In some variations, source 12 of animal data 14 refers to data related to targeted individual 16ⁱ. Targeted individual 16ⁱ is the subject from which corresponding animal data 14 is collected. Label i is merely an integer label from 1 to iₘₐₓ associated with each targeted individual where iₘₐₓ is the total number of targeted individuals, which can be 1 to several thousand to several million or more. In this context, animal data can refer to any data related to a subject. In a refinement, targeted individual 16ⁱ can be a known individual, an assumed individual (e.g., presumed individual, an individual who has identified themselves as a particular individual without verification of that individual's identity), or an unknown individual, with the identity of the targeted individual determined by the comparison between at least one created, modified, or enhanced unique asset and the animal data derived from the one or more source sensors, or its one or more derivatives (e.g., one or more unique assets). In another refinement, a known subject can be an assumed subject and vice versa (i.e., an assumed subject can be a known subject). In some embodiments, animal data refers to data related to a subject's body derived, at least in part, from one or more sensors and, in particular, biological sensors (also referred to as biosensors). Therefore, the one or more sources 12 of animal data includes one or more sensors. In many useful applications, targeted individual 16ⁱ is a human (e.g., an athlete, a soldier, a healthcare patient, a research subject, a participant in a fitness class, a video gamer) and the animal data 14^{k} is human data.

Animal data 14^{k} can be derived from a targeted individual 16 or multiple targeted individuals 16ⁱ (e.g., including a targeted group of multiple targeted individuals, multiple targeted groups of multiple targeted individuals). In the case of sensors that collect data from one or more targeted individuals 16ⁱ, the animal data 14^{k} can be obtained from a single sensor gathering information from each targeted individual 16ⁱ, or from multiple sensors gathering information from each targeted individual 16ⁱ. Each sensor 18 gathering animal data from source 12 of animal data 14^{k} from targeted individual 16ⁱ can be classified as a source sensor. In some cases, a single sensor can capture data from multiple targeted individuals, a targeted group of multiple targeted individuals, or multiple targeted groups of multiple targeted individuals (e.g., an optical-based camera sensor that can locate and measure distance run for a targeted group of targeted individuals, interpret biomechanical movements, capture visual images of the targeted individuals, use facial recognition software to identify individuals, and the like). Each sensor can provide a single type of animal data or multiple types of animal data. In a variation, sensor 18 can include multiple sensing elements to measure one or more parameters within a single sensor (e.g., heart rate and accelerometer data). One or more sensors 18 can collect data from a targeted individual 16ⁱ engaged in a variety of activities including strenuous activities that can change one or more biological signals or readings in a targeted individual such as blood pressure, heart rate, or biological fluid levels. Activities may also include sedentary activities such as sleeping or sitting where changes in biological signals or readings may have less variance. One or more sensors 18 can also collect data after one or more other activities (e.g., after a run, after waking up, after ingesting one or more substances or medications, and any other activity suitable for data collection from one or more sensors). In a refinement, one or more sensors 18 can be classified as a computing device with one or more computing capabilities. In a variation, animal data-based identification and recognition system 10 can also gather (e.g., receive, collect) animal data not obtained from sensors (e.g., animal data that is inputted or gathered via a computing device; animal data sets that include artificial data values not generated directly from a sensor; animal data received from another computing device). This can occur via computing device 20 or via one or more other computing devices that gather animal data. In a refinement, at least one sensor of the one or more source sensors captures two or more types of animal data. In another refinement, one or more sensors 18 are operable to collect at least a portion of non-animal data. In another refinement, one or more sensors can capture information related to one or more other sensors. In another refinement, at least one sensor of the one or more source sensors 18 is comprised of two or more sensors. In another refinement, the one or more sensors 18 can collect data over a continuous period of time or at regular or irregular intervals.

One or more sensors 18 can include one or more biological sensors (also referred to as biosensors). Biosensors collect biosignals, which in the context of the present embodiment are any signals or properties in, or derived from, animals that can be continually or intermittently measured, monitored, observed, calculated, computed, or interpreted, including both electrical and non-electrical signals, measurements, and artificially-generated information. A biosensor can gather biological data (including readings and signals) such as one or more of physiological data, biometric data, chemical data, biomechanical data, genetic data, genomic data, glycomic data, location data, or other biological data (i.e., animal data) from one or more targeted individuals. Moreover, it should be appreciated that, unlike typical biometric analysis, the data analysis (e.g., comparison) provided herein can be used for multiple purposes and not just for identification and verification of the targeted subject. For example, the analysis provided herein additionally allow for risk mitigation, detection of fraudulent behavior, optimization of a target individual's performance and health, and the other examples provided herein. In particular, a biosensor can gather biological data (including readings and signals) such as physiological data and/or biological fluid data (e.g., blood) and/or chemical data, which may vary over time or under a variety of conditions for a targeted individual thereby making the association of such data with the individual difficult. The methods herein allow the identification of the targeted subject, the targeted medical condition, or the targeted biological response in such situations, and in particular, that the targeted subject is the person actually wearing such sensors. In a refinement, sensors prone to vary over time can be used with biological data that tends not to vary so much over time (e.g., fingerprint). For example, some biosensors may measure, or provide information that can be converted into or derived from, biological data such as eye tracking & recognition data (e.g., pupillary response, movement, pupil diameter, iris recognition, retina scan, eye vein recognition, EOG-related data), blood flow data and/or blood volume data (e.g., PPG data, pulse transit time, pulse arrival time), biological fluid data (e.g., analysis derived from blood, urine, saliva, sweat, cerebrospinal fluid), body composition data (e.g., bioelectrical impedance analysis, weight-based data including weight, body mass index, body fat data, bone mass data, protein data, basal metabolic rate, fat-free body weight, subcutaneous fat data, visceral fat data, body water data, metabolic age, skeletal muscle data, muscle mass data), pulse data, oxygenation data (e.g., SpO2), core body temperature data, galvanic skin response data, skin temperature data, perspiration data (e.g., rate, composition), blood pressure data (e.g., systolic, diastolic, MAP), glucose data (e.g., fluid balance I/O, glycogen usage), hydration data (e.g., fluid balance I/O), heart-based data (e.g., heart rate, average HR, HR range, heart rate variability, HRV time domain, HRV frequency domain, autonomic tone, ECG-related data including PR, QRS, QT, RR intervals, echocardiogram data, thoracic electrical bioimpedance data, transthoracic electrical bioimpedance data), neurological data and other neurological-related data (e.g., EEG-related data), genetic-related data, genomic-related data, skeletal data, muscle data (e.g., EMG-related data including surface EMG, amplitude, adenosine triphosphate (ATP) data, muscle fiber types, muscle contraction velocity, muscle elasticity, soft-tissue strength), respiratory data (e.g., respiratory rate, respiratory pattern, inspiration/expiration ratio, tidal volume, spirometry data), and the like. Some biosensors may detect biological data such as biomechanical data which may include, for example, angular velocity, joint paths, kinetic or kinematic loads, gait description, step count, reaction time, or position or accelerations in various directions from which a subject's movements may be characterized. Some biosensors may gather biological data such as location and positional data (e.g., GPS, ultra-wideband RFID-based data; posture data), facial recognition data, posterior profiling data, audio data, kinesthetic data (e.g., physical pressure captured from a sensor located at the bottom of a shoe), other biometric authentication data (e.g., fingerprint data, hand geometry data, voice recognition data, keystroke dynamics data—including usage patterns on computing devices such as mobile phones, signature recognition data, ear acoustic authentication data, eye vein recognition data, finger vein recognition data, footprint and foot dynamics data, body odor recognition data, palm print recognition data, palm vein recognition data, skin reflection data, thermography recognition data, speaker recognition data, gait recognition data, lip motion data), or auditory data (e.g., speech/voice data, sounds made by the subject) related to the one or more targeted individuals. Some biological sensors may be image or video-based and collect, provide and/or analyze video or other visual data (e.g., still or moving images, including video, MRIs, computed tomography scans, ultrasounds, echocardiograms, X-rays) upon which biological data can be detected, measured, monitored, observed, extrapolated, calculated, or computed (e.g., biomechanical movements or location-based information derived from video data, a fracture detected based on an X-Ray, or stress or a disease of a subject observed based on a video or image-based visual analysis of a subject). Some biosensors may derive information from biological fluids such as blood (e.g., venous, capillary), saliva, urine, sweat, and the like including (but not limited to) triglyceride levels, red blood cell count, white blood cell count, adrenocorticotropic hormone levels, hematocrit levels, platelet count, ABO/Rh blood typing, blood urea nitrogen levels, calcium levels, carbon dioxide levels, chloride levels, creatinine levels, glucose levels, hemoglobin A1c levels, lactate levels, sodium levels, potassium levels, bilirubin levels, alkaline phosphatase (ALP) levels, alanine transaminase (ALT) levels, and aspartate aminotransferase (AST) levels, albumin levels, total protein levels, prostate-specific antigen (PSA) levels, microalbuminuria levels, immunoglobulin A levels, folate levels, cortisol levels, amylase levels, lipase levels, gastrin levels, bicarbonate levels, iron levels, magnesium levels, uric acid levels, folic acid levels, vitamin B-12 levels, and the like. In a variation, some biosensors may collect biochemical data including acetylcholine data, dopamine data, norepinephrine data, serotonin data, GABA data, glutamate data, hormonal data, and the like. In addition to biological data related to one or more targeted individuals, some biosensors may measure non-biological data (e.g., ambient temperature data, humidity data, elevation data, and barometric pressure data, and the like). In a refinement, one or more sensors provide biological data that include one or more calculations, computations, measurements, predictions, probabilities, possibilities, estimations, evaluations, inferences, determinations, deductions, observations, or forecasts that are derived, at least in part, from animal data. In another refinement, the one or more biosensors are capable of providing at least a portion of artificial data. In another refinement, the one or more biosensors are capable of providing two or more types of data, at least one of which is biological data (e.g., heart rate data and VO2 data, muscle activity data, and accelerometer data, VO2 data and elevation data).

In a refinement, the animal data derived from the one or more source sensors and utilized to identify and verify the targeted subject is further utilized for another one or more purposes that provide consideration (e.g., monetary, non-monetary) or another form of value to the targeted subject or other gatherer of animal data (e.g., animal data acquirer). For example, the animal data derived from the one or more source sensors used to identify and verify the targeted individual is also used to mitigate one or more risks, detect fraudulent activity, as information to create one or more insurance products or adjust a premium, as one or inputs to monitor and optimize human body-based performance, as an asset that can be exchanged for consideration, and the like. Characteristically, this is different from other systems (e.g., facial recognition authentication systems, fingerprint authentication systems) that utilize the animal data for a single purpose (e.g., identify or verify the individual) without any value creation occurring from the animal data itself.

In another refinement, at least one sensor 18 and/or its one or more appendices thereof can be affixed to, are in contact with, or send one or more electronic communications in relation to or derived from, one or more targeted subjects including the one or more targeted subjects' body, skin, eyeball, vital organ, muscle, hair, veins, biological fluid, blood vessels, tissue, or skeletal system, embedded in one or more targeted subjects, lodged or implanted in one or more targeted subjects, ingested by one or more targeted subjects, or integrated to include at least a subset of one or more targeted subjects. For example, a saliva sensor affixed to a tooth, a set of teeth, or an apparatus that is in contact with one or more teeth, a sensor that extracts DNA information derived from a targeted subject's biological fluid or hair, sensor that is wearable (e.g., on a human or other animal body), a sensor in a computing device (e.g., phone) that is tracking a targeted individual's location information or collecting other biometric information (e.g., facial recognition, voice, fingerprint), one or more sensors integrated within a head-mountable unit such as smart glasses or a virtual/augmented/mixed reality headset that track eye movements and provide eye tracking data and recognition data, one or more sensors that are integrated into one or more computing devices that analyze biological fluid data, a sensor affixed to or implanted in the targeted subject's brain that may detect brain signals from neurons, a sensor that is ingested by a targeted subject to track one or more biological functions, a sensor attached to, or integrated with, a machine (e.g., robot) that shares at least one characteristic with an animal (e.g., a robotic arm with an ability to perform one or more tasks similar to that of a human; a robot with an ability to process information similar to that of a human), and the like. Advantageously, the machine itself can include one or more sensors, and may be classified as both a sensor and a subject. In another refinement, the one or more sensors 18 are integrated into or as part of, affixed to, or embedded within, a textile, fabric, cloth, material, fixture, object, or apparatus that contacts or is in communication with a targeted individual either directly or via one or more intermediaries or interstitial items. Examples include, but are not limited to, a sensor attached to the skin via an adhesive, a sensor integrated into a watch or head-mountable or wearable unit (e.g., augmented reality or virtual reality headset, smart glasses, hat, headband), a sensor integrated or embedded into clothing (e.g., shirt, jersey, shorts, wristband, socks, compression gear), a sensor integrated into a steering wheel, a sensor integrated into a computing device controller (e.g., video game or virtual environment controller, augmented reality headset controller, remote control for media), a sensor integrated into a ball that is in contact with the targeted subject's hands (e.g., basketball), a sensor integrated into a ball that is in contact with the targeted subject's feet (e.g., soccer), a sensor integrated into a ball that is in contact with an intermediary being held by the targeted subject (e.g., bat), a sensor integrated into a hockey stick or a hockey puck that is in intermittent contact with an intermediary being held by the targeted subject (e.g., hockey stick), a sensor integrated or embedded into the one or more handles or grips of fitness equipment (e.g., treadmill, bicycle, row machine, bench press, dumbbells), a sensor that is integrated within a robot (e.g., robotic arm) that is being controlled by the targeted individual, a sensor integrated or embedded into a shoe that may contact the targeted individual through the intermediary sock and adhesive tape wrapped around the targeted individual's ankle, and the like. In another refinement, one or more sensors may be interwoven into, embedded into, integrated with, or affixed to, a flooring or ground (e.g., artificial turf, grass, basketball floor, soccer field, a manufacturing/assembly-line floor, yoga mat, modular flooring), a seat/chair, helmet, a bed, an object that is in contact with the targeted subject either directly or via one or more intermediaries (e.g., a subject that is in contact with a sensor in a seat via a clothing intermediary), and the like. In another refinement, one or more sensors may be integrated with or affixed to one or more aerial apparatus such as an unmanned aerial vehicle (e.g., drone, high-altitude long-endurance aircraft, a high-altitude pseudo satellite (HAPS), an atmospheric satellite, a high-altitude balloon, a multirotor drone, an airship, a fixed-wing aircraft, or other altitude systems) or other aerial computing device that utilize one or more sensors (e.g., optical, infrared) to collect animal data (e.g., skin temperature, body temperature, heart rate, heart rate variability, respiratory rate, facial recognition, gait recognition, location data, image data, one or more subject characteristics or attributes, and the like) from one or more targeted subjects or groups of targeted subjects. In another refinement, the sensor and/or its one or more appendices may be in contact with one or more particles or objects derived from the targeted subject's body (e.g., tissue from an organ, hair from the subject) from which the one or more sensors derive, or provide information that can be converted into, biological data. In yet another refinement, one or more sensors may be optically-based (e.g., camera-based) and provide an output from which biological data can be detected, measured, monitored, observed, extracted, extrapolated, inferred, deducted, estimated, determined, combined, calculated, or computed. In yet another refinement, one or more sensors may be light-based and use infrared technology (e.g., temperature sensor or heat sensor) to gather or calculate biological data (e.g., skin or body temperature) from an individual or the relative heat of different parts of an individual. In yet another refinement, the one or more sensors gather animal data related to one or more medical conditions, biological responses, or attributes/characteristics of an individual (e.g., an optical sensor that gathers animal data such as skin color, facial hair, eye color, conditions of the skin, and the like).

In a variation depicted in FIG. 1, at least one sensor 18 gathers animal data 14^{k} from each targeted individual 16ⁱ. The at least one sensor 18 can provide the information (e.g., animal data 14^{k}) to one or more computing devices 20 or another computing device (e.g., intermediary server 22, cloud server 40). In a variation, computing device 20 can operate one or more programs to gather animal data 14^{k} (e.g., import animal data, enable one or more subjects to input animal data, communicate with at least one sensor 18 to gather animal data, and the like), one or more characteristics/attributes related to the one or more targeted individuals 16ⁱ (e.g., characteristics/attributes such as age, weight, height, eye color, skin color, hair color (if any), birthdate, race, nationality, habits, medical history, family history, medication history, financial history, and the like), non-animal data, or a combination thereof (e.g., a subset, any combination of subsets, or all). For the purposes of this invention, any reference to either the term “characteristic” or “attribute” should be interpreted as encompassing all possible meanings of both terms. In some variations, computing device 20 can be operable to gather information from a single targeted individual or multiple targeted individuals (e.g., including one or more groups of targeted individuals), as in the case of a hospital that uses a computing device to manage multiple patients, an insurance company or fitness organization that uses a computing device to manage multiple individuals, a sports team utilizing a computing device to manage its players, a holding company utilizing a computing device to manage groups of employees across one or more portfolio companies, and the like. In another variation, one or more intermediary servers 22 or cloud servers 40 can operate one or more programs to gather animal data 14^{k} related to the one or more targeted individuals 16ⁱ, one or more characteristics/attributes related to the one or more targeted individuals 16ⁱ, non-animal data, or a combination thereof. The one or more intermediary servers 22 or cloud servers 40 can be operable to gather animal data 14^{k} or other information from one or more sensors 18, one or more computing devices 20, each other (e.g., intermediary server 22 can be operable to gather information from cloud server 40 and vice versa), other computing devices (e.g., computing device 25), or a combination thereof. Therefore, computing device 20, intermediary server 22, and cloud server 40 can each be the collecting computing device(s) described herein. One or more intermediary servers 22 or cloud servers 40 can be operable to gather information from a single targeted individual or multiple targeted individuals (e.g., including one or more groups of targeted individuals).

Still referring to FIG. 1, animal data 14^{k} gathered by the one or more computing devices can include attached thereto individualized metadata, which may include one or more characteristics/attributes related to the animal data, including characteristics/attributes related to the one or more sensors, (e.g., sensor type, sensing type, sensor model, sensor brand, firmware information, sensor positioning on or related to a subject, operating parameters, sensor properties, sampling rate, mode of operation, data range, gain, time stamps, other sensor settings, and the like), characteristics/attributes of the one or more targeted individuals, origination of the animal data, type of animal data, source computing device of the animal data, data format, algorithms used, quality of the animal data, speed at which the animal data is provided, and the like. Metadata can also be associated with (e.g., attached to, included as part of, affiliated with, grouped with, linked to) the animal data after it is collected. Metadata can also include any set of data that describes and provides information about other data, including data that provides context for other data (e.g., the activity a targeted individual is engaged in while the animal data is collected, the location in which the animal data was collected, and the like; in some examples, animal data provides context for other animal data, such as the cadence at which a subject was pedaling their stationary bicycle for an acquirer who wants heart rate data for stationary-based cycling activities), rules related to the data (e.g., how the data can be used, permissions and/or restrictions related to use of the data, other terms and/or conditions related to use of the data), and the like. It can also include information such as how the animal data has been previously used, previous acquirers of the animal data, where and when the animal data has been previously sent, previous acquisition costs or values of the animal data, guidelines related to use of the data, information related to the one or more targeted individuals, and the like. In some variations, such information may be contained in one or more digital records directly or indirectly associated with the animal data, the one or more targeted individuals, or both. Depending on the type of data, metadata may also be classified as animal data or non-animal data. In a refinement, animal data includes metadata that incorporates one or more attributes related to the targeted individual.

Other information, including one or more characteristics/attributes of one or more targeted individuals from which the animal data originated or other characteristics/attributes related to the one or more sensors or animal data, can be added to the metadata (e.g., included as metadata) or associated with the animal data (e.g., as metadata) upon collection of the animal data or at a later time after the animal data is collected (e.g., upon identification and/or verification of the one or more individuals). It can also be gathered by one or more programs operated by computing device 20 or associated computing devices (e.g., intermediary server 22, cloud server 40). Examples of a targeted individual's one or more attributes can include, but are not limited to, name, age, weight, height, birthdate, race, eye color, skin color, hair color (if any), reference identification (e.g., social security number, national ID number, digital identification) country of origin, area of origin, ethnicity, current residence, addresses, phone number, gender of the targeted individual from which the animal data originated, data quality assessment, and the like. In a refinement, the targeted individual's attributes can also include information (e.g., animal data) gathered from medication history, medical history, medical records, health records, genetic-derived data, genomic-derived data, (e.g., including information related to one or more medical conditions, traits, health risks, inherited conditions, drug responses, DNA sequences, protein sequences and structures), biological fluid-derived data (e.g., blood type), drug/prescription records, allergies, family history, health history (including mental health history), manually-inputted personal data, physical shape (e.g. body shape), historical personal data, and the like. The targeted individual's one or more attributes can also include one or more activities the targeted individual is engaged in while the animal data is collected, one or more associated groups (e.g., if the individual is part of a sports team, or assigned to a classification based on one or more medical conditions), one or more habits (e.g., tobacco use, alcohol consumption, exercise habits, nutritional diet, the like), education records, criminal records, financial information (e.g., bank records, such as bank account instructions, checking account numbers, savings account numbers, credit score, net worth, transactional data), social data (e.g., social media accounts, social media history, records, internet search data, social media profiles, metaverse profiles, metaverse activities/history), employment history, marital history, relatives or kin history (in the case the targeted subject has one or more children, parents, siblings, and the like), relatives or kin medical history, relatives or kin health history, manually inputted personal data (e.g., one or more locations where a targeted individual has lived, emotional feelings, mental health data, preferences), historical personal data, and/or any other individual-generated data. In a refinement, one or more characteristics/attributes associated with another one or more subjects can be associated with one or more targeted individuals. For example, in the event the targeted individual has children, the subject's (i.e., child's) health condition may be associated with the one or more targeted individuals as a characteristic associated with the one or more targeted individuals' data (e.g., if the child is sick, the parent may be under considerable stress or have deteriorating mental health which may impact their animal data). In another example, the one or more characteristics/attributes of the targeted individual's avatar or representation in a virtual environment, video game, or other simulation (e.g., including their actions, experiences, conditions, preferences, habits, and the like) may be associated with the targeted individual and may be included as part of the targeted individual's animal data. In another refinement, animal data is inclusive of the targeted individual's one or more attributes and/or characteristics (i.e., the one or more characteristics/attributes can be categorized as animal data). In another refinement, the one or more characteristics/attributes provides context for other data (e.g., animal data). In another refinement, at least a portion of gathered data can be classified as both animal data and metadata. In another refinement, the system may associate metadata with one or more types of animal data prior to its collection (e.g., the system may collect one or more attributes related to the targeted individual prior to the system collecting animal data and associate the one or more attributes in the targeted individual's profile to the one or more types of animal data prior to its collection).

It should be appreciated that the animal data and/or various attributes related to of the animal data can be anonymized or de-identified (e.g., pseudonymized) by the system. De-identification involves the removal or alteration of personal identifying information in order to protect personal privacy. In the context of the present invention, a reference to one of the terms (i.e., anonymized or de-identified) should include reference to both terms and similar terms (e.g., semi-anonymized, partially-anonymized) where applicable, and a reference to one of the terms should not be interpreted as limiting but rather as encompassing all possible meanings of the terms where applicable. In a refinement, the system does not require identification of the targeted subject in order to receive sensor-based animal data from one or more sensors over a continuous or intermittent time period (e.g., regular or irregular intervals; point-in-time readings). Advantageously, the system can anonymize (or semi-anonymize), or receive/utilize anonymized (or semi-anonymized) reference animal data that can identify one or more medical conditions or biological responses without subject identification. For example, the system may receive anonymized or partially-anonymized data in which it is unable to identify the targeted individual yet still be operable to identify one or more biological responses or medical conditions via the data. Similarly, individuals may not want their identity known as part of a reference animal data set, yet allow their animal data and/or one or more attributes/characteristics to be shared in order to enhance the system's ability to identify one or more medical conditions or biological responses based upon the reference animal data. This can be advantageous for individuals who do not want to share their identity but would still like to know if their bodies are showing signs of a medical condition or biological response, or individuals who want to provide their de-identified data to a database of reference animal data that can provide information for other subjects to identify a medical condition or biological response.

Still referring to FIG. 1, computing device 20 includes an operating system that coordinates interactions between one or more types of hardware and software. In a refinement, computing device 20 mediates the sending of animal data 14^{k} to intermediary server 22 or cloud server 40, i.e., it collects the animal data from one or more sensors 18, as well as from programs operating on computing device 20 that gather animal data, and transmits the animal data to (or makes available to) intermediary server 22, cloud server 40, or a combination thereof. For example, computing device 20 can be a smartphone, wrist mountable unit (e.g., smart watch), a head-mountable unit (e.g., smart glasses, virtual reality or augmented reality headset), smart glasses, a desktop computer, a laptop computer, or any other type of computing device. In some cases, computing device 20 is local to the targeted individual, although not required. In another refinement, one or more sensors 18 may be housed within, attached to, affixed to, or integrated with, computing device 20 (e.g., as in the case of a computing device such as a smart watch, smart glasses, smart clothing, hearables, smart contact lens, augmented or virtual reality headset, any other bodily-mountable unit, and the like which include one or more sensors 18 that collects animal data). In this variation, computing device 20 may also be categorized as sensor 18 (e.g., one or more camera-based sensors in a mobile computing device such as a smartphone; one or more sensors collecting physiological, location, and/or biomechanical data in a mobile computing device such as a smart watch; and the like). In some variations, the functionality of computing device 20 can be deployed across multiple computing devices (e.g., multiple computing devices execute the one or more functionalities, actions, programs, or a combination thereof, of computing device 20). In a refinement, computing device 20 can include multiple computing devices 20.

It should be appreciated that both cloud server 40 and intermediary server 22 can include a single computer server or a plurality of interacting computer servers. In this regard, intermediary server 22 and cloud server 40 can communicate with one or more other systems—including each other—to monitor, receive, and record the one or more requests or distributions related to animal data, as well as gather, action upon, and distribute animal data, non-animal data, or a combination thereof. In a refinement, intermediary server 22 and cloud server 40 can be operable to communicate with one or more other systems—including each other—to monitor, receive, and record one or more uses or requested uses related to animal data. In a refinement, one or more computing devices 20, intermediary servers 22, or cloud servers 40 may include be one or more unmanned aerial vehicles that perform one or more of the functions or actions of computing device 20, intermediary server 22, cloud server 40, or a combination thereof. Additional details related to an unmanned aerial vehicle-based animal data collection and distribution system are disclosed in U.S. Pat. No. 10,980,218 filed Jul. 19, 2019 and U.S. patent Ser. No. 16/977,570 filed Sep. 2, 2020; the entire disclosures of which are hereby incorporated by reference.

In a variation, intermediary server 22 (e.g., local server or other type of server) communicates directly with the source of animal data 14^{k}, as shown by one or more communication links 34 with one or more sensors 18 or by one or more communication links 36 with one or more computing devices 20. In another variation, cloud server 40 communicates directly with the source of animal data 14^{k}, as shown by one or more communication links with one or more sensors 18 or by one or more communication links with one or more computing devices 20. In a refinement, intermediary server 22 communicates with the source 12 of animal data 14^{k} through a cloud server 40 or other local server. Cloud server 40 can be one or more servers that are accessible via the internet or other network. Cloud server 40 can be a public cloud, a hybrid cloud, a private cloud utilized by the organization operating intermediary server 22, a localized or networked server/storage, localized storage device (e.g., n terabyte external hard drive or media storage card), or distributed network of computing devices. In a refinement, cloud server 40 includes multiple cloud servers. In another refinement, intermediary server 22 includes multiple intermediary servers. In another refinement, intermediary server 22 operates as cloud server 40. In another refinement, cloud server 40 operates as intermediary server 22. In another refinement, both cloud server 40 and intermediary server 22 are utilized in animal data-based identification and recognition system 10. In another refinement, at least one cloud server 40 or intermediary server 22 is utilized in animal data-based identification and recognition system 10.

Still referring to FIG. 1, one or more individuals 19ⁱ (e.g., reference individuals) are the one or more subjects from which reference animal data 21 corresponds with. One or more individuals 19ⁱ can include one or more targeted individuals 16ⁱ, as well as other individuals with associated animal data. In the case of targeted individual 16ⁱ, once their animal data 14^{k} is collected (or accessible) and identified and/or verified by the system as being derived from (or associated with) the targeted individual, or associated with a medical condition or biological response, animal data 14^{k} can become reference animal data 21, with the system operable to collect data in real-time, in near real-time, or over a period of time (e.g., minutes, hours, days, weeks months, years, and the like) from one or more source sensors and/or one or more computing devices. Other data associated with the animal data 14^{k} (e.g., contextual data, other metadata) may be provided with animal data 14^{k} when it is gathered by computing device 25 to be included as reference animal data 21. In a variation, other data associated with the animal data 14^{k} may be added to the reference animal database and associated with the animal data after it is provided (e.g., the database of reference animal data 21 is updated as new information is comes in). In some variations, animal data can be classified as both animal data 14^{k} and reference animal data 21.

Reference animal data 21 can be any reference animal data that is directly or indirectly related to one or more individuals 19ⁱ. Data from the one or more individuals 19ⁱ comprise the database of reference animal data 21 (e.g., accessible via one or more computing devices 25) from which the one or more known, assumed, or unknown individuals are identified. Characteristically, reference animal data 21 for each individual 19ⁱ can include one or more changes or variations in the animal data (e.g., in the signals or readings of the animal data), enabling the system to create, modify, or enhance one or more digital records (e.g., profiles) for each individual 19ⁱ that provides information related to their biological-based patterns, rhythms, signatures, identifiers, trends, features, measurements, outliers, abnormalities, anomalies, characteristics, and the like based on one or more variables. The one or more variables can include contextual data (e.g., metadata) such as age, medical fitness, time, environmental conditions, location, activity (e.g., is the subject sitting of standing; did the subject recently finish a run), characteristics/attributes, stimuli provided to the individual 19ⁱ that results in one or more animal data signals or readings (e.g., is the individual responding to a specific stimulus or stimuli that results in the signals or readings derived from the animal data), and the like. In a refinement, the one or more variables induce one or more changes or variations in the individual's animal data. In another refinement, the one or more variables influence (e.g., have a material impact on) one or more biological phenomena in, or derived from, the individual capable of being converted to electrical signals that can be captured by one or more sensors. In another refinement, the one or more variables induce one or more unique responses (e.g., physiological responses or other type of biological data-based responses) by the body that can be calculated, computed, derived, extracted, measured, extrapolated, quantified, simulated, estimated, evaluated, inferred, established, deduced, or observed via the animal data and captured via one or more sensors. The combination of the one or more variables, as well as the combination of animal data based upon the one or more variables, enables more unique patterns, rhythms, signatures, identifiers, trends, features, measurements, outliers, abnormalities, anomalies and characteristics (i.e., unique assets) to be derived. This information enables the system to create, modify, or enhance one or more baselines (e.g., known comparison data) for each individual 19ⁱ, one or more medical conditions, and/or one or more biological responses from which one or more unique assets can be derived.

The one or more digital records are included as part of the reference animal data 21 database and can include each individual 19ⁱ's animal data and other information (e.g., attributes, other metadata), representing that individual's reference animal data 21. In a refinement, the one or more digital records may be created, modified, or enhanced for one or more medical conditions and/or biological responses (e.g., a digital record is created which includes reference animal data for all individuals who suffered a heart attack within n number of days of having a stroke, enabling the system to identify characteristics in stroke patients that could predict if the individual is likely to have a heart attack or not). In another refinement, the same reference animal data 21 is included as part of two or more digital records.

In another refinement, the database of reference animal data 21 can be distributed across one or more databases on one or more computing devices. In another refinement, reference animal data from each individual 19ⁱ or subset of individuals 19ⁱ may comprise one or more databases, the totality of which comprises the database of reference animal data 21. In another refinement, a plurality of databases comprise the database of reference animal data 21. In another refinement, one or more previously-created unique assets for an individual or group of individuals may be included as part of the reference animal database and gathered by the system to make one or more comparisons with the animal data derived from one or more source sensors, or its one or more derivatives.

Reference animal data 21 can be gathered (e.g., inputted, imported, collected) from one or more individuals 19ⁱ by one or more computing devices 25. One or more computing devices 25 can be one or more computing devices from which the reference animal data 21 is gathered, stored, transformed, or made available (e.g., distributed, accessed). One or more computing devices 25 can operate as a separate one or more computing devices with different functionalities as one or more computing devices 20, clouds 40, or intermediary servers 22, or it can operate as separate computing device with one or more shared functionalities as one or more computing devices 20, clouds 40, or intermediary servers 22. In a refinement, the one or more computing devices 25 can operate as one or more computing devices 20, clouds 40, or intermediary servers 22. In another refinement, the one or more computing devices 25 are one or more computing devices 20, clouds 40, or intermediary servers 22.

The gathered reference animal data 21 can be derived from one or more sensors 18 or from one or more computing device 25 via one or more other computing devices (e.g., one or more computing devices 20, clouds 40, or intermediary servers 22, or third-party systems 42). For example, computing device 20 may operate an application that enables a targeted individual to input animal data into the application (e.g., how the targeted individual is feeling, symptoms, daily routine information, nutrition information, other attributes, and the like), which can be gathered by one or more computing device 25 to become reference animal data 21. Reference animal data 21 can be accessed by a single computing device or multiple computing devices. In many variations, access from multiple computing devices can occur simultaneously. In a refinement, reference animal data 21 can be gathered by one or more computing devices 25 from one or more data acquirers 26 or other external sources (e.g., one or more third-party computing devices 42). In another refinement, the reference animal data 21 gathered from one or more computing devices has attached metadata that enables the reference animal data 21 to be associated with one or more individuals 19ⁱ, medical conditions, biological responses, or a combination thereof (e.g., via one or more digital records).

In a variation, reference animal data 21 can be gathered by one or more computing devices 25 from one or more other computing devices, one or more sensors, or a combination thereof. Reference animal data 21 can be gathered, stored, transformed, made available, or a combination thereof, by a single computing device 25 or across multiple computing devices 25. In some variations, the one or more computing devices that gather reference animal data 21 may be different from the one or more computing devices that store the reference animal data 21 or make available the reference animal data 21 (e.g., to create, modify, or enhance the at least one unique asset). In other variations, the one or more computing devices that gather the reference animal data 21 may be same as the one or more computing devices that store and/or make available the reference animal data.

Characteristically, animal data 14^{k} from one or more individuals 16ⁱ and one or more sensors 18 can be collected by one or more computing devices 20, intermediary servers 22, clouds 40, or a combination thereof, and provided to one or more computing devices 25 as reference animal data 21 once the animal data is associated with the one or more individuals 16ⁱ. Reference animal data 21 can also include other data related to one or more individuals 19ⁱ provided by one or more computing devices (e.g., computing device 20, intermediary server 22, cloud 40, third-party computing device 42). In a refinement, animal data 14^{k} is accessible as reference animal data 21 only after one or more identifications or verifications occur (e.g., the system first identifies or verifies that animal data 14^{k} is derived from or associated with one or more targeted individuals, medical conditions, or biological responses prior to including it as reference animal data 21).

Still referring to FIG. 1, at least one unique asset 23 is created, modified, or enhanced from reference animal data 21. The at least one unique asset 23 can be created, modified, or enhanced from reference animal data 21 by one or more computing devices 25. One or more computing devices 25 are operable to create, modify, or enhance the at least one unique asset 23 from one or more calculations, computations, measurements, derivations, extractions, extrapolations, simulations, creations, combinations, modifications, enhancements, estimations, evaluations, inferences, establishments, determinations, conversions, deductions, or observations based upon the reference animal data 21 that enable the identification of one or more targeted individuals, medical conditions, or biological responses. In a refinement, the at least one unique asset 23 enables identification of one or more sensors or sensor characteristics. In another refinement, the at least one unique asset 23 is created, modified, or enhanced from reference animal data 21 on a single computing device 25. In another refinement, the at least one unique asset 23 is created, modified, or enhanced from reference animal data 21 on two or more computing devices. In another refinement, the at least one unique asset 23 is created, modified, or enhanced via one or more sensors (e.g., one or more unmanned aerial vehicles or other computing apparatus with one or more sensors integrated or attached and computing capabilities to create, modify, or enhance the at least one unique asset). In another refinement, one or more unique assets 23 are included as part of reference animal data 21. In this example, the unique asset can be associated with the one or more individuals 19ⁱ, medical conditions, biological responses, or a combination thereof, in the database of reference animal data via inclusion in their corresponding one or more digital records. In a variation, the one or more unique assets 23 included as part of reference animal data 21 can be modified (e.g., updated based upon new data entering the system), enhanced, or removed by the system. In another refinement, the one or more computing devices 25 take one or more of the following actions (e.g., processing steps) on the collected reference animal data 21 to transform the reference animal data into at least one unique asset 23: normalize, timestamp, aggregate, clean, tag, store, manipulate, denoise, process, enhance, organize, analyze, visualize, simulate, anonymize, synthesize, summarize, replicate, productize, or synchronize the data. In another refinement, one or more computing devices 20, intermediary servers 22, clouds 40, or a combination thereof, access reference animal data 21 in order to create, modify, or enhance one or more unique assets 23.

The at least one unique asset 27 can be created, modified, or enhanced from animal data 14^{k} by one or more computing devices 20, intermediary servers 22, or clouds 40 via one or more calculations, computations, measurements, derivations, extractions, extrapolations, simulations, creations, combinations, modifications, enhancements, estimations, evaluations, inferences, establishments, determinations, conversions, deductions, or observations. Characteristically, the at least one unique asset 27 is created, modified, or enhanced from animal data 14^{k} without inclusion of reference animal data 21 as part of the unique asset. However, in some cases, the one or more computing devices may direct how the at least one unique asset 23 and/or the at least one unique asset 27 are derived based upon what one or more types of animal data 14^{k} are collected from the one or more sensors and/or what type of reference animal data 21 has been gathered by computing device 25 (e.g., the system may make a determination regarding the type of unique asset to create based upon the commonality of animal data collected from one or more sensors 18 and gathered by computing device 25 as part of the database of reference animal data, as well as other data characteristics that may include quality of data, quantity of data, and the like). In a refinement, the system determines the type of unique asset created based upon one or more requests, an evaluation of the reference animal data 21 (e.g., evaluation of the data available), an evaluation of animal data 14^{k}, an evaluation of the one or more sensors that derive animal data 14^{k}, an evaluation of the metadata, or a combination thereof. The one or more requests can be determined or defined by the use case (e.g., an insurance company or animal data marketplace company may want to verify the identity of the individual wearing the source sensor, while a hospital may want to determine whether an individual has a specific medical condition or any medical condition contained in the reference animal data). In another refinement, the at least one unique asset 27 can be created, modified, or enhanced from animal data 14^{k} via one or more sensors. In another refinement, the at least one unique asset 27 is created, modified, or enhanced from animal data 14^{k} on a single computing device (e.g., computing device 20, cloud 40, intermediary server 22). In another refinement, the at least one unique asset 27 is created, modified, or enhanced from animal data 14^{k} on two or more computing devices. In another refinement, the one or more computing devices take one or more of the following actions (e.g., processing steps) on the collected animal data 14^{k} to transform the animal data into at least one unique asset 27: normalize, timestamp, aggregate, clean, tag, store, manipulate, denoise, process, enhance, organize, analyze, visualize, simulate, anonymize, synthesize, summarize, replicate, productize, or synchronize the data.

In a refinement, the one or more unique assets are comprised of one or more identifiable patterns, rhythms, trends, features, measurements, outliers, abnormalities, anomalies, data sets, characteristics/attributes, or a combination thereof, that are derived from one or more biological phenomena that occur within a subject, the one or more biological phenomena capable of being converted to electrical signals that can be captured, at least in part, by one or more sensors and converted into animal data. Characteristically, the one or more biological phenomena can change or vary based upon one or more variables, leading to the same type of animal data to have different readings based upon one or more variables. The one or more variables are included with the animal data as metadata. In this context, metadata can include contextual data, which provides the context for the collected data (e.g., where was the data collected, in what activity was the data collected in, what were the environmental conditions, and the like), characteristics/attributes of the individual, characteristics related to the one or more sensors, and the like. The system identifies and records the one or more changes or variations in the animal data based upon the one or more variables and creates one or more digital records that enables the system to associate the animal data, the metadata (e.g., the one or more variables), and the one or more changes or variations with the individual, a medical condition, and/or a biological response. The collection of the one or more changes and variations in the animal data based upon information found in the metadata can create one or more identifiable patterns, rhythms, trends, features, measurements, outliers, abnormalities, anomalies, data sets, characteristics/attributes, or a combination thereof, that are unique to the individual, the medical condition, or biological response. This information is included in the database of reference animal data (i.e., reference animal database). Furthermore, the system is operable to identify the one or more changes or variations in the animal data derived, at least in part, from one or more source sensors in real-time or near real-time. Upon gathering animal data from the one or more source sensors and associated metadata (e.g., contextual data which can include the context in which the animal data was collected and information related to the one or more variables), the system can identify the individual, the medical condition, or the biological response by matching the one or more changes or variations found in the one or more digital records in the reference animal database (with each change or variation including metadata associated with it to provide context to the change or variation) with the changes or variations found in the animal data derived from the one or more source sensors based upon the metadata.

For example, the system may collect a plurality of animal data simultaneously from one or more sensors and identify one or more patterns in a subject's ECG, heart rate variability data, and breathing rate data in light of the one or more variables (e.g., context in which the data was collected, the one or more characteristics/attributes of the individual, and the like, the information of which is included in the metadata). The system collects the animal data and metadata and identifies the one or more patterns—which may be different based on the one or more variables—in a variety of contexts to create one or more digital records for the individual, a medical condition, and/or biological response as part of the reference animal database. The collection of the one or more patterns in each of the animal data types comprises the one or more unique assets created for the individual, the medical condition, or the biological response. The system then collects data from one or more source sensors in a real-time or near real-time manner. Depending on what the system is looking for (e.g., is the system identifying an individual, a medical condition, a biological response, or a combination there), the system identifies (or looks for) one or more patterns in the collected animal data, and evaluates the metadata. The one or more patterns derived from the animal data comprises the one or more unique assets. The system then takes the one or more patterns from the animal data collected from the one or more source sensors (e.g., via the unique asset) and matches them with the one or more patterns in the reference animal database (e.g., via the reference unique asset). The match between the two unique assets identifies the individual, the medical condition, the biological response, or a combination thereof. In a refinement, the system may derive one or more insights, predictive indicators, or a combination thereof, from the at least one unique asset, the animal data derived from the one or more source sensors (or its one or more derivatives), or a combination thereof, from which one or more identifications or verifications occur (e.g., the system may evaluate the one or more patterns from the two or more unique assets and derive an insight that enables identification of verification to occur; the system may evaluate the one or more patterns from the two or more unique assets and make a prediction based upon the likelihood of positive identification or verification occurring).

Still referring to FIG. 1, one or more intermediary servers 22, cloud servers 40, or a combination thereof can communicate either directly or indirectly with one or more third-party computing devices 42 via one or more communication links 44. Third-party computing device 42 is any computing device (e.g., which includes systems/programs operating on that computing device) that can gather information (e.g., receive or collect animal data) provided by another computing device either directly or indirectly, or provide information (e.g., animal data, metadata) related to one or more subjects. The one or more third-party computing devices 42 are typically the acquirers of the animal data. One or more third-party computing devices 42 can include, but are not limited to, sports media systems (e.g., for displaying the collected data), sports wagering or other wagering-affiliated systems, e-sports and video gaming systems, insurance provider/underwriting systems, telehealth systems, health analytics systems, risk analytics systems (e.g., insurance, finance), performance analytics systems, corporate wellness systems, health and wellness monitoring systems (e.g., including systems to monitor viral infections, electronic medical record systems, electronic health records systems, and the like), research systems, security systems, subject verification systems (e.g., digital passport systems, media or content platforms), authentication systems, fitness systems, military systems, hospital systems, pharmaceutical systems, emergency response systems, financial systems, banking systems, social media platforms, relationship management systems (e.g., dating application), simulation systems (e.g., virtual environment systems), and the like. It can also include systems located on the one or more targeted individuals (e.g., a wearable sensor with a display such as a smart watch, smart glasses, or virtual reality/augmented reality headset) or other individuals interested in accessing the one or more targeted individuals' data (e.g., a sports bettor interested in accessing the animal data from one or more targeted individual athletes on their computing device such as their mobile computing device, or a sports betting operator interested in verifying the integrity of the one or more competing athletes based upon their animal data via the one or more unique assets). In a refinement, one or more sensors 18 are operable to communicate either directly or indirectly (e.g., via computing device 20, intermediary server 22, or cloud 40) with one or more third-party computing devices 42. In another refinement, one or more computing devices 20 are operable to communicate either directly or indirectly (e.g., via intermediary server 22, cloud 40, or sensor 18) with one or more third-party computing devices 42. In another refinement, one or more computing devices 25 are operable to communicate either directly or indirectly with one or more third-party computing devices 42. In another refinement, one or more third-party computing devices 42 operate in conjunction with computing device 20, cloud server 40, intermediary server 22, computing device 25, or a combination thereof, as part of a single animal data-based identification and recognition system. In another refinement, one or more third-party computing devices 42 operate one or more programs on computing device 20, cloud server 40, intermediary server 22, computing device 25, or a combination thereof, to gather and/or evaluate animal data, reference animal data, or a combination thereof.

In another refinement, intermediary server 22 provides animal data 24 (e.g., which can include one or more insights, predictive indicators, computed assets, and derivatives of animal data including one or more unique assets, metadata and/or non-animal data associated with the animal data, and the like) to a third party such as data acquirer 26 (e.g., via one or more computing devices 26) for consideration (e.g., payment, a reward, a trade for something of value which may or may not be monetary in nature, which can include adjustment on insurance premiums, healthcare services costs, and the like. A non-monetary example is a free or discounted insight or predictive indicator that has value to the provider in exchange for the provider's animal data or a free or discounted sensor in exchange for the provider's animal data, or digital tokens with no cash value but valuable to the provider, or other benefit). For example, a sports betting operator offering bets on a sports competition may acquire an insight that verifies the integrity of the one or more competing athletes in a professional sports competition based upon (at least in part) their animal data—via the one or more unique assets—for consideration to ensure the one or more athletes are not intentionally “throwing a match.” Animal data 24 can include any data derived from, or associated with, one or more individuals included as part of reference animal data 21, animal data 14^{k}, or other associated data (e.g., metadata or non-animal data associated with animal data 14^{k}, the one or more individuals, or both). In another refinement, the intermediary server 22 distributes at least a portion of the consideration to at least one stakeholder 30 (e.g., computing device 30). The one or more stakeholders can be a user that produced (e.g., generated) the data (e.g., the targeted subject from which the animal data is derived), the owner of the data (which may be different from the individual that generated the data), the data collection company, authorized distributor of the animal data, a sensor company (e.g. a sensor company that collected the acquired animal data), an analytics company (e.g., an analytics company that provided analytics on the acquired data or curated the data), an application company, a data visualization company, an intermediary server company that operates the intermediary server, a cloud server company that operates the cloud server, a company that operates one or more computing devices that stores or provides access to the reference animal data (which is used to verify the association between the targeted individual and their animal data), or any other entity (e.g., typically one that provides value to any of the aforementioned stakeholders or the data acquirer). In another refinement, cloud 40 or computing device 20 operate as intermediary server 22. In another refinement, one or more data acquirers 26 or stakeholders 30 are also one or more third-party computing devices 42 and vice versa. In another refinement, the one or more computing devices associated with data acquirer 26 are represented by one or more third-party computing devices 42 (i.e., the one or more third-party computing devices 42 operate as the one or more computing devices utilized by data acquirer 26 to acquire animal data).

Still referring to FIG. 1, computing device 20 can gather animal data 14^{k} from source 12 via one or more communication links either wirelessly, via one or more wired connections, or a combination thereof. Computing device 20 may include a hardware transmission subsystem that enables electronic communication with one or more sources 12 of animal data 14^{k}. In some variations, the hardware transmission subsystem can include one or more receivers, transmitters, transceivers, and/or supporting components (e.g., dongle) that utilize a single antenna or multiple antennas, which may be configured as part of a mesh network and/or utilized as part of an antenna array. The transmission subsystem and/or its one or more components may be housed within the one or more computing devices or may be external to the computing device (e.g., a dongle connected to the computing device which includes one or more hardware and/or software components that facilitates wireless communication and is part of the transmission subsystem). In a refinement, one or more components of the transmission subsystem and/or one or more of its components are integral to, included within, or attached to, the one or more sensors 18. Computing device 20 may also include one or more network connections, such as an internet connection or cellular network connection, which may include hardware and software aspects, or pre-loaded hardware and software aspects that do not necessitate an internet connection. In a refinement, one or more sensors 18 or intermediary servers 22 operate as computing device 20. In a variation, the one or more users interact with one or more sensors 18 or intermediary servers 22 in replace of at least a portion of the functionality of computing device 20. In another refinement, one or more sensors 18 or intermediary servers 22 take on one or more functions or features of computing device 20. In another refinement, one or more sources 12 of animal data 14^{k} transmits the animal data to a computing device (e.g., computing device 20, intermediary server 22, cloud 40) via the hardware transmission subsystem. In another refinement, computing device 20 is operable to collect animal data from multiple sensors. In another refinement, one or more computing devices are operable to collect animal data (e.g., including reference animal data) from one or more other computing devices.

In a variation, the hardware transmission subsystem can communicate electronically with the one or more sensors 18 from the one or more targeted individuals 16ⁱ using one or more wireless methods of communication via one or more communication links 34. In this regard, animal data-based identification and recognition system 10 can utilize any number of communication protocols and conventional wireless networks to communicate with one or more sensors 18 including, but not limited to, Bluetooth Low Energy (BLE), ZigBee, cellular networks, LoRa, ultra-wideband, Ant+, WiFi, and the like. The present invention is not limited to any type of technologies or electronic communication links (e.g., radio signals) the one or more sensors 18 or any other computing device utilized to transmit and/or receive signals. Advantageously, the transmission subsystem enables the one or more sensors 18 to transmit data wirelessly for real-time or near real-time communication. In this context, near real-time means that the transmission is not purposely delayed except for necessary processing by the sensor and any of the one or more computing devices taking one or more actions on or with the data. In another variation, one or more apparatus with one or more onboarded computing devices (e.g., such as an aerial apparatus like an unmanned aerial vehicle or other remote computing device) may act as a transmission subsystem to collect and distribute animal data from one or more sensors or other information from one or more targeted subjects or groups of targeted subjects. In a refinement, the one or more apparatus may have one or more sensors attached, or integrated, as part of the apparatus to collect animal data.

Still referring to FIG. 1, animal data-based identification and recognition system 10 may gather information from the one or more sensors (e.g., animal data) in one of two ways: (1) the system communicates directly with a sensor, thereby bypassing any native system that is associated with the sensor; or (2) the system communicates with the cloud or native system associated with the sensor, or other computing device that provides access to the sensor data, via an API or other data transfer mechanism in order to provide the data to the system. However, the present invention is not limited by the multitude of ways in which animal data may be gathered from the one or more sensors by the system. In a variation, communication between the system and the one or more sensors may be a two-way communication where the system can receive one or more signals (e.g., biosignals, other readings) from, and send one or more signals (e.g., commands, instructions, information) to, the one or more sensors. For example, the system may send one or more commands to the one or more sensors to change one or more functionalities of a sensor (e.g., change the gain, power mode, or sampling rate, start/stop streaming, update the firmware) or operate in defined ways (e.g., a user can define the data collection period and communicate such operating parameters to the sensor; the computing device may be programmed to automatically select the type, volume, and/or frequency of animal data the system wants to collect from a subject based upon the one or more sensors being utilized in order to create the one or more unique assets, which may in part be based upon an assessment of the reference animal data available; the system can send a command to the sensor to take an action, such as vibrate on the body of the individual, in order to induce one or more biological-based responses from the body of the individual that can be captured via one or more sensors to uniquely identify/verify the individual). In some cases, a sensor may have multiple sensors within a device (e.g., accelerometer, gyroscope, ECG, etc.) which may be controlled, at least in part, by the system. This includes one or more sensors being turned on or off, and increasing or decreasing sampling frequency or sensitivity gain. Advantageously, the system's ability to communicate directly with the one or more sensors also enables real-time or near real-time collection of the sensor data from the sensor to the system. Direct sensor communication can be achieved by either creation or modification of one or more lines of code to communicate with the sensor or the sensor manufacturer writes code to function with the system. It can be achieved via a wireless or wired connection. The system may create a standard for communication with the system that one or more sensor manufacturers may follow. Furthermore, the system may have the ability to control any number of sensors, any number of functionalities, and stream any number of sensors on any number of targeted individuals through the single program. In a refinement, the system is operable to create and send one or more commands simultaneously to multiple sensors.

In a refinement, the system may establish two-way communication with the one or more sensors and communicate directly with the one or more sensors that are capturing animal data from the one or more subjects to confirm that the one or more characteristics of the one or more source sensors matches at least one characteristic of the animal data (e.g., to verify that the animal data was derived from the one or more source sensors), with the one or more characteristics of the one or more source sensors including at least one of: identity of the sensor, sensor type, sensing type, sensor model, sensor brand, firmware information, sensor positioning, operating parameters, sensor properties, sensor settings, sampling rate, mode of operation, data range, or gain.

Still referring to FIG. 1, computing device 20 can include a display device that enables a user (e.g., a subject utilizing one or more sensors from which animal data is collected; an administrator operating the system on behalf of a subject utilizing one or more sensors from which animal data is collected, and the like) to take one or more actions within the display (e.g., touch-screen enabling an action; use of a scroll mouse that enables the user to navigate and make selections; voice-controlled action via a virtual assistant or other system that enables voice-controlled functionality; eye-tracking within spatial computing systems that enables an eye-controlled action; a neural control unit that enables one or more controls based upon brain waves; and the like). In a refinement, a gesture controller that enables limb (e.g., hand) or body movements to indicate an action may be utilized to take one or more actions. In another refinement, the display may act as an intermediary to communicate with another one or more computing devices to execute the one or more actions requested by the user.

Typically, a display device communicates information in visual form. Information may include information related to the animal data, instructions provided to a user to take one or more actions as part of one or more software programs (e.g., instructions to enable the system to collect animal data from the user and create one or more unique assets), one or more stimuli that induce a biological-based response that can be captured via the one or more sensors to identify/verify the individual, and the like. The display device can also provide an ability for the user to communicate information with the system (e.g., ability for a user to provide one or more inputs to operate the program, provide requested information to the system, and the like). However, a display device may communicate and/or receive information to a user utilizing one or more other mechanisms including via an audio or aural format (e.g., verbal communication of information such as biological readings), via a physical gesture (e.g., a physical vibration which provides information related to the one or more biological readings, a physical vibration which indicates when the data collection period is complete, or a physical gesture to induce a biological-based response from the individual's body can be captured as animal data via one or more sensors), or a combination thereof. In some variations, the information communicated to or provided by a user may be animal data-based information such as the type of animal data, activity associated with the animal data or other metadata, insights or predictive indicators, and the like. For example, the display device may not communicate the signals or readings associated with the animal data for the user to interact with but may communicate the type of animal data (e.g., the display may not provide a user's actual heart rate values but may display the term “heart rate” or “HR” or a symbol related to heart rate—such as a heart—which the user can select and define terms related to their heart rate data). In another refinement, the display may not include any visual component in its communication or receipt of information (e.g., as in the case of a smart speaker, hearables, or similar computing device that does not include any visual screen to interact with and is operable via a virtual or audio-based assistant to receive one or more commands and take one or more actions. In this example, the smart speaker or hearables may be in communication with another computing device to visualize information via another display if required).

A display device may include a plurality of display devices (or displays) that comprise the display device. In addition, a display that is not included as part of computing device 20 may be in communication with computing device 20 (e.g., attached or connected to, from which communication occurs either via wired communication or wirelessly). Furthermore, the display device may take one or more forms. Examples of where one or more types of animal data may be displayed include via one or more monitors (e.g., via a desktop, laptop computer, projector), holography-based computing devices, smart phone, tablet, a smart watch or other wearable with an attached or associated display, smart speakers (e.g., including earbuds/hearables), smart contact lens, smart clothing, smart accessories (e.g., headband, wristband), or within a head-mountable unit (e.g., smart glasses or other eyewear/headwear including virtual reality/augmented reality headwear) where the animal data (e.g., computed asset, insight, predictive indicator, and the like) or other animal data-related information can be visualized or communicated. In a refinement, the display may be operating as part of, or displaying/receiving animal data or animal data-related information (or other information requested by the system) via of one or more programs that include or are related to, but not limited to, a fitness system (e.g., a home fitness or gym application that enables users to view or access their animal data), health passport system, animal data monetization system (e.g., including systems for providing loans using animal data as collateral, at least in part, or as part of an animal data-based digital currency system), insurance system, wagering system (e.g., sports wagering system), animal performance system (e.g., human performance optimization system), telehealth system, health analytics system, electronic medical records system, electronic health records system, risk analytics system (e.g., insurance, insurance underwriting, finance, security), pharmaceutical-based system (e.g., drug administration system), performance analytics system, health and wellness monitoring system (e.g., including systems to monitor viral infections), research system, security/integrity system (e.g., subject or sensor identification/verification/authentication for security purposes; system that identifies and/or verifies fraudulent behavior), military system, hospital system, emergency response system, financial system, banking system, relationship management system, social media system, simulation/video game system (e.g., virtual world, metaverse), media & entertainment system, and the like. In another refinement, the display may include one or more other media streams (e.g., live-stream video, digital objects). For example, a home fitness machine (e.g., cycling machine) may include an integrated display that enables both the visualization of media (e.g., video of a fitness instructor) along with the real-time animal data, or a computing device may be operating health monitoring program (e.g., telehealth application) which may include an integrated media module (e.g., real-time video of a doctor or medical professional with two-way video and voice communication) within the display alongside the real-time animal data being communicated (e.g., visualized) by the system, or a virtual environment may that includes a variety of digital objects may also incorporate animal data or animal data-based information in the virtual world, and the like.

In one variation, the one or more computing devices can provide a display for a user (e.g., which may be the targeted subject or another user such as an administrator operating the system on behalf of the targeted subject) to notify the system of the assumed (e.g., presumed) identity of a targeted subject and/or their associated one or more source sensors. In some variations, the identity of the targeted subject may not be assumed but rather known or unknown. It may also be assumed or unknown that one or more source sensors are associated with one or more targeted individuals (e.g., collecting animal data from the one or more targeted individuals), thus requiring a form of identification and/or verification (e.g., the targeted individual may inform the system that the one or more sensors are collecting data from the targeted individual when in fact the one or more sensors are collecting data from another individual, thus requiring the system to make one or more identifications and/or verifications). The display device can enable a user to provide information (e.g., via search function, input function, or other mechanism that provides information to a computing device) related to the one or more targeted subjects and/or source sensors to the one or more computing devices. For illustration purposes, information can include one or more characteristics/attributes related to the one or more targeted subjects (e.g., name) the source sensors (e.g., identity of each sensor associated with each targeted individual), the animal data (e.g., one or more symptoms or health-related inputs related to one or more medical conditions—which may be known, assumed, or unknown), the metadata (e.g. a biological response such as the activity the targeted subject is engaged in while animal data is being collected), and the like. In a refinement, the one or more computing devices includes the collecting computing device that gathers animal data from the one or more source sensors. In another refinement, the collecting computing device is configured to source the reference animal data. In another refinement, the collecting computing device also collects the animal data from the one or more computing devices.

The user can access an animal data collection program (e.g., which may be an insurance-based program, healthcare-based program, data monetization program, security program, any of the aforementioned use cases, or any similar use cases) via the display using one or more identification techniques (e.g., a login page requiring a password; biometric authentication such as a fingerprint scan, facial/retina recognition, voice recognition, and the like) to identify the user (and/or targeted subject, if different). In some variations, the user accessing the animal data collection program may be the targeted subject. In other variations, the user may not be the targeted subject but an administrator operating the system on behalf the targeted subject, with the targeted subject in proximity of the system or the one or more sensors to initiate data collection. Upon accessing the program, the user or system may initiate the system's collection of animal data from the one or more source sensors associated with the one or more targeted subject whereby the one or more source sensors are communicating with the system to provide animal data from a subject—presumably the targeted subject. In order to establish communication, the user can select or confirm—via the animal data collection program—the one or more source sensors being utilized by the targeted subject to initiate animal data collection via one or more input or selection functions that enable the user to associate the one or more source sensors with the targeted individual. In some variations, the selection or confirmation of the one or more source sensors may occur based upon automatic detection by the system (e.g., the system may identify one or more source sensors within proximity of the computing device for the user to confirm or select as being associated with the targeted subject). Upon confirmation or selection, the system may establish communication with the one or more source sensors that are associated with the targeted subject to receive animal data. At this point, while the user (e.g., which may or may not be the targeted subject) has identified themselves through one or more identification techniques to access the program, the system has no way of verifying that the one or more source sensors associated with the targeted subject are, in fact, collecting data from the targeted subject. It is assumed (or presumed) by the system that the subject utilizing (e.g., wearing, using) the one or more source sensors is in fact the targeted subject; however, using a wearable sensor as an example, the targeted subject may have placed the wearable sensor on another one or more subjects after verifying their identity to log into the system (if the targeted subject is also the user) and prior to initiating the system's collection of animal data. A targeted subject may take this action, for example, if providing their data to a system (e.g., insurance-based, health-based, animal data-monetization based) may be detrimental to their economic or social benefit (e.g., associating someone else's data with their profile may be more economically beneficial, particularly in the case of insurance adjustments, health-based checkups, and the like). In this example, with the wearable source sensor on another subject, the other subject's animal data would be associated with the targeted subject within the system instead of animal data collected from the targeted subject. This can cause issues for systems (e.g., insurance-based, health-based, animal data-monetization based) wanting to collect animal data and accurately associate it with (e.g., assign it to) the correct targeted individual.

In a variation, a targeted subject (e.g., targeted subject x) may log in or provide one or more identifiable information inputs into a program for collecting animal data and initiate communication with the one or more source sensors associated with the targeted subject (e.g., which may be one or more wired source sensors connected to the one or more computing devices, one or more wireless source sensors, or a combination thereof) to initiate data collection. At this point, it is assumed by the system that the subject is targeted subject x, but there is no mechanism to verify that the one or more source sensors that are in communication with the system are, in fact, collecting data from targeted subject x. In the case of on-body source sensors, there is no mechanism to verify that the one or more source sensors that are in communication with the system are, in fact, on the body of targeted subject x or collecting animal data from targeted subject x (e.g., another subject alongside targeted subject x may be utilizing the one or more source sensors for animal data collection instead of targeted subject x; another subject may have the same one or more sensors as targeted subject x and targeted subject x may have assigned the other subject's one or more sensors as the one or more source sensors associated with targeted subject x; targeted subject x may have two of the same sensors—one of which is collecting animal data and providing it to the system and one of which is non-functional—and wear the non-functional sensor to provide visual confirmation to the system while providing the data-collecting sensor to another subject, enabling association of the other subject's animal data with the targeted subject).

As a solution, an animal data identification and recognition system enables identification of a targeted subject, medical condition (e.g., including any disease, illness or injury; any pathologic, mental or psychological condition or disorder; non-pathologic conditions that normally receive medical treatment), or biological response based upon their gathered animal data from one or more source sensors. In one embodiment, identification of a subject, medical condition or biological response (e.g., the activity the subject is undertaking, bodily response or biological phenomenon capable of being converted to electrical signals that can be captured by one or more sensors including a biological state—such as stress—or activities in the body; a medical event such as a heart attack, stroke; anomalies in biological patterns or rhythms; an injury; and the like) occurs with a targeted subject in mind (e.g., a subject has been inputted into or provided to the system as the targeted subject the system is identifying/verifying). Upon the system implementing one or more data collection programs to collect animal data via one or more source sensors from a subject (e.g., which may or may not be the targeted subject, the identity of which may be known or unknown; in a variation, the identity may be assumed or presumed—e.g., we assume it is subject x), the system creates, modifies, or enhances at least one unique asset for the targeted subject, the targeted medical condition, or the targeted biological response derived from reference animal data. The system then evaluates (e.g., compares, analyzes) the at least one created, modified, or enhanced unique asset derived from the reference animal data with the animal data derived from one or more source sensors, or its one or more derivatives (e.g., a unique asset created, modified, or enhanced by the system and derived from the collected sensor-based animal data) from the subject—who or which may be assumed or unknown—to identify whether the subject is in fact the targeted subject or not, to identify whether the subject has the targeted medical condition or not, or to identify the biological response related to the subject. The identification of one or more medical conditions or biological responses may occur with a known subject. In the case of an initial unknown subject, medical condition, or biological response, the evaluation (e.g., comparison) between the at least one created, modified, or enhanced unique asset derived from the reference animal data and the animal data derived from the one or more source sensors, or its one or more derivatives (e.g., one or more unique assets), identifies the targeted subject, targeted medical condition, or targeted biological response. In the case of an initial assumed targeted subject (e.g., assumed subject, stated subject, presumed subject without verification that is, in fact, the targeted subject), the comparison between the at least one created, modified, or enhanced unique asset and the animal data derived from the one or more source sensors, or its one or more derivatives, verifies the identification of the targeted subject, targeted medical condition, or targeted biological response.

In a refinement, in the case of an unknown condition of a targeted subject, the system creates one or more unique assets for each known condition based upon the reference animal data. The system then utilizes the gathered animal data from the one or more source sensors and creates one or more unique assets to identify whether the subject has any of the one or more known conditions to identify the unknown condition as a known condition. In another refinement, with an assumed or known condition of a targeted subject, the system creates one or more unique assets for the assumed or known condition based upon the reference animal data. The system then utilizes the gathered animal data from the one or more source sensors and creates one or more unique assets to identify and/or verify whether the subject has the assumed condition. It should be appreciated that the same (or materially similar) methodologies can be applied for identifying and/or verifying unknown, assumed, or known identifies of targeted subjects, as well as unknown, assumed, or known biological responses.

It should be also appreciated that one or more described features for any one of the embodiments may be included as a feature for any of the one of the other embodiments. In addition, “identification of a targeted subject” can be inclusive of identifying (e.g., determining) one or more characteristics or attributes related to the targeted subject, including but not limited to, age, weight, height, eye color, skin color, hair color (if any), gender, ethnicity, race, country of origin, area of origin, one or more habits (e.g., tobacco use, alcohol consumption, sleep, lifestyle, exercise habits, nutritional diet, food habits, technology consumption), and the like. The term “identify” is inclusive of the term “recognize” and vice versa. In some embodiments, identifying can also include “determining.” In a refinement, “identify” can also mean “not recognize” or “not identify” as in the case of a system not identifying a targeted subject, medical condition, or biological response (e.g., the system communicating that the two or more unique assets do not match; therefore the subject is not the targeted subject, the targeted subject does or does not have the medical condition, there is no match for the medical condition, there is no match for the biological response, or the inputted biological response is not accurate). Additionally, the term “related to” in the context of a targeted subject includes directly or indirectly derived from a targeted subject (e.g., animal data captured from a subject using one or more sensors, derivatives created based upon the captured animal data from the targeted subject), animal data that may not be derived from a targeted subject but can be applied to the targeted subject based upon one or more shared attributes or characteristics with the targeted subject (or other animal) or their animal data, and the like. Similar criteria can be applied for the one or more medical conditions and biological responses.

In another embodiment, identification of a subject, medical condition, or biological response occurs with a targeted subject in mind. The system creates, modifies, or enhances at least one unique asset for the targeted subject, the one or more targeted medical conditions, or the one or more targeted biological responses from reference animal data related to the targeted subject, the one or more targeted medical conditions, or the one or more targeted biological responses. The system implements one or more data collection programs to collect animal data via one or more source sensors from a subject (e.g., which may or may not be the targeted subject; the identity may be known or unknown; the identify may be assumed, presumed, and the like). The system then evaluates (e.g., compares) the at least one created, modified, or enhanced unique asset with the collected animal data from one or more source sensors, or its one or more derivatives (e.g., one or more unique assets, insights, predictive indicators, computed assets, and the like), from the subject to identify: (1) whether the unknown or assumed subject is, in fact, the targeted subject (or not); (2) whether the known, unknown, or assumed subject has the one or more targeted medical conditions (or not); (3) the one or more biological responses related to the known, unknown, or assumed subject; or (4) a combination thereof. In the case of an initial unknown subject, medical condition, or biological response, the evaluation of (or comparison between) the at least one created, modified, or enhanced unique asset derived from the reference animal data and the animal data derived from the one or more source sensors, or its one or more derivatives, identifies the targeted subject, the one or more targeted medical conditions, or the one or more targeted biological responses. In the case of an initial assumed subject (e.g., assumed subject, presumed subject, stated subject), the comparison between the at least one created, modified, or enhanced unique asset and the animal data derived from the one or more source sensors, or its one or more derivatives, verifies the identification of the targeted subject, the one or more targeted medical conditions, or the one or more targeted biological responses. In the case of a known subject, the comparison between the at least one created, modified, or enhanced unique asset and the animal data derived from the one or more source sensors, or its one or more derivatives, verifies the identification of the one or more targeted medical conditions or the one or more targeted biological responses.

In a refinement, if the subject is an assumed targeted individual, the system may first create the unique asset from the reference animal data for that individual to verify the identity of the individual. If the subject is unknown individual, or in the event the system does not verify the identity the assumed targeted individual, the system may create the unique asset from the reference animal data for each of an initial subset of reference individuals (e.g., known individuals) to identify the individual. The subset of reference individuals may be selected by the system based upon (ii) one or more types of metadata available for both the reference animal data and the animal derived from the one or more source sensors; (ii) one or more characteristics/attributes of the individual and shared with the reference individuals; or (iii) one or more other searchable parameters. In the event the system does not identity of the individual based on an initial subset of reference individuals, the system may broaden the scope of animal data being evaluated within the initial subset of reference individuals, change the one or more parameters (e.g., removing parameters, broadening parameters, and the like) to broaden the subset of reference individuals, or a combination thereof. Similar methodologies can be applied for evaluating one or more medical conditions and/or biological responses.

In another refinement, the at least one unique asset derived from the one or more source sensors is created, modified, or enhanced with a medical condition or biological response in mind (i.e., the at least one unique asset is created, modified, or enhanced based upon one or more pre-identifiable traits, patterns, identifiers, and the like that are associated specifically with the medical condition or biological response, which are compared with one or more unique assets derived from the reference animal data and associated specifically with medical condition or biological response to determine whether the targeted individual in fact has that medical condition or is exhibiting that biological response). In a refinement, the at least one unique asset derived from the one or more source sensors is created, modified, or enhanced with a plurality of medical conditions or biological responses in mind. In this scenario, the unique asset derived from the one or more source sensors may include a plurality of pre-identifiable traits, patterns, identifiers, and the like for multiple medical conditions or biological responses that enable the system to identify one or more biological responses or medical conditions. In another refinement, the at least one unique asset derived from the one or more source sensors is created, modified, or enhanced without a specific medical condition or biological response in mind. In this scenario, the system may take the animal data derived from the one or more source sensors and create one or more unique assets (e.g., a collection of animal data and non-animal data-based information related to the targeted subject, which may include one or more insights, trends, patterns, identifiers, and the like) and cross-reference the targeted individual's information with information associated with one or more medical conditions or biological responses in the reference animal data database to identify the one or more medical conditions or biological responses. For example, the system may create the unique asset from the one of more source sensors based upon one or more generally accepted identifiers for a variety of diseases. The unique asset can then be used and searched against a variety of medical conditions in the reference animal database that may identify more of more medical conditions. It should be appreciated that similar methodologies as described above can be applied for identifying one or more subjects.

In some variations, the type of animal data being collected by the one or more source sensors, the one or more characteristics of the one or more sensors (e.g., type of sensor, sampling rate, and the like), the metadata, or a combination thereof, may dictate what reference animal data is being accessed and/or utilized by the system in order to create the one or more unique assets. The system may also provide one or more instructions to the user to ensure the right animal data or metadata is being collected (e.g., to ensure the subject is using the correct source sensor(s), to ensure the subject is using the correct sensor parameter(s)) in order to enable the one or more identifications or verifications to occur. In a refinement, the system may automatically provide one or more commands to the one or more source sensors (e.g., to configure the one or more sensors), other sensors, or other computing devices in order to collect the requisite data to identify the one or more targeted subjects, medical conditions, or biological responses.

In another variation, the identity of the targeted subject, medical condition, or biological response may be unknown. In these cases, the system can identify one or more subjects, medical conditions, or biological responses based on one or more evaluations (e.g., comparisons) of the two or more unique assets, at least one of which is derived from the animal data gathered by the one or more source sensors. In one embodiment, identification of one or more targeted individuals occurs by first creating, modifying, or enhancing at least one unique asset for n number of known individuals (e.g., hundreds, thousands, millions, billions, and the like) based upon collected reference animal data for the known individuals, which is accessible via their one or more digital records in the reference animal database. In most cases of identifying a targeted individual, there will only be one unique asset for each individual (e.g., the most accurate, reliable, and repeatable unique asset at any given time) based upon the available animal data at any given time. However, in some cases, there may potentially be multiple unique assets depending on the type of data that has been collected (e.g., to enable comparison across a broader range of subjects if different types of data has been collected), one or more characteristics related to the one or more source sensors used to collect the animal data, and the like. Therefore, the type of unique asset created can a tunable parameter. The system gathers the animal data from the one or more source sensors from the targeted subject and creates, modifies, or enhances at least one unique asset for the targeted subject. The system then compares the at least one created, modified, or enhanced unique asset from each of the one or more known subjects with the at least one created, modified, or enhanced unique asset from the targeted subject. Characteristically, the system is operable to make multiple comparisons simultaneously or in succession. The comparison between the two or more unique assets identifies the targeted subject. In a variation, identification can be characterized by at least one of: a percentage match, possibility, probability, prediction, confidence indicator (e.g., degree of confidence), score (e.g., accuracy score, precision score, and the like), or likelihood (e.g., 78% likelihood that the targeted individual is a specific known subject). Characteristically, an identification can include a positive identification (e.g., 100% match), partial positive identification, meaning the identification is not absolute (e.g., n % match that is less than 100%), or non-identification (e.g., the system verifies that the animal data is not derived from the targeted subject). For example, the system can be operable to create one or more unique assets for a plurality of known individuals (e.g., reference individuals)—at least one unique asset for each known individual—and compare the unique asset of the targeted individual derived from their animal data gathered from the one or more source sensors with the unique assets of other known individuals derived from the reference animal data to identify the targeted subject. In another variation, one or more common characteristics between the two or more unique assets identifies the targeted subject. In a refinement, at least a portion of the animal data from the identified targeted subject, or its one or more derivatives, is distributed by one or more computing devices to one or more other computing devices for consideration.

In some variations, the data in the reference animal database may not be uniform—each individual 19ⁱ may have different types of animal data collected from different types of sensors, including different metadata associated with the animal data (e.g., data captured in non-uniform environments, different characteristics/attributes for each reference individual), different quantities of data, and the like. In addition, subject 16ⁱ may be utilizing one or more source sensors or sensors operating parameters that provide animal data that is different—at least in part—from data located in the reference animal data. To solve for this problem, the system can utilize one or more artificial intelligence techniques to take one or more actions upon the animal data derived from the one or more source sensors, associated metadata, reference animal data, or a combination thereof, to create, modify, or enhance the one or more unique assets based upon: (1) the type of animal data being collected by the one or more source sensors; (2) the one or more types of sensors; (3) the one or more operating parameters or characteristics associated with each source sensor; (4) the metadata (e.g., one or more external factors including activity in which data is collected, time, environmental conditions, location, and the like); (5) the types of animal data in the reference animal database and its associated metadata; (6) the sources of animal data in the reference animal database; or (7) a combination thereof. In a refinement, the one or more actions including at least one of: normalize, timestamp, aggregate, clean, tag, store, manipulate, denoise, process, enhance, organize, analyze, visualize, simulate, anonymize, synthesize, summarize, replicate, productize, or synchronize the data. This will enable the system to derive one or more unique assets that are common across at least a portion of the n number of known individuals and the unknown targeted individual in order to facilitate comparison across individuals. In a refinement, the system may send one or more communications to the one or more source sensors associated with the unknown targeted individual, which may allow the system to gather different types of animal data, or gather animal data with different characteristics (e.g., at a different sampling rate or featuring different metadata), or other information to derive at least one unique asset.

In another embodiment, identification of one or more known medical conditions or biological responses (e.g., a biological response such as activity or behavior of an individual, or the “stress” level of an individual, or a biological phenomenon that is a precursor to a medical episode or event such as a heart attack) occurs by first creating, modifying, or enhancing at least one unique asset for n number of medical conditions or biological responses based upon collected reference animal data, with each condition or response having potentially multiple unique assets (e.g., STEMI, NSTEMI, coronary spasm, unstable angina as being types of heart attacks, each potentially having their own one or more unique assets), and sub-conditions within conditions (e.g., type 1 diabetes and type 2 diabetes as sub-conditions of diabetes) having one or more unique assets. In a refinement, the at least one unique asset can identify sub-conditions of the one or more medical conditions. In another refinement, the at least one unique asset can identify sub-responses of the one or more biological responses (e.g., degrees of a response—e.g., walking slow vs walking fast). The system gathers (i) the animal data from the one or more source sensors from the targeted subject, and (2) the at least one created, modified, or enhanced unique asset—derived from the reference animal data—for each of the one or more known medical conditions or biological responses. For clarification purposes, a known medical condition or biological response can include any medical condition or biological response with one or more unknown causes, features, or characteristics but having at least one identifiable pattern, trend, rhythm, feature, measurement, characteristic, outlier, anomaly, and the like from which at least one unique asset can be created, modified, or derived. Characteristically, the system creates, modifies, or enhances the at least one unique asset from the collected animal data based upon the pre-identified signatures, patterns, rhythms, trends, features, measurements, outliers, abnormalities, anomalies, data sets, characteristics/attributes, or other identifiers in animal data that enables identification of each of the one or more medical conditions or biological responses. The system then compares the at least one created, modified, or enhanced unique asset for the one or more known medical conditions or biological responses with the at least one unique asset from the targeted subject, and the comparison between the at least two unique assets identifies the one or more medical conditions or biological responses. In a variation, one or more common characteristics between the at least two unique assets identifies the one or more medical conditions or biological responses. In a refinement, at least a portion of the animal data or its one or more derivatives from the identified one or more medical conditions or biological responses (via the one or more source sensors) is distributed by one or more computing devices to one or more other computing devices for consideration.

In a variation, the comparison between the at least one created, modified, or enhanced unique asset and the animal data derived from the one or more source sensors, or its one or more derivatives, that identifies the targeted subject, targeted medical condition, or targeted biological response occurs between at least two unique assets, at least one of which is a created, modified, or enhanced unique asset from the animal data derived from the one or more source sensors. In this variation, at least two unique assets are compared to identify the targeted subject, targeted medical condition, or targeted biological response. In another variation, the comparison to identify the targeted subject, the targeted medical condition, or the targeted biological response occurs between two or more unique assets, at least one of which is a created, modified, or enhanced unique asset from the animal data derived from the one or more source sensors. In a refinement, the two or more unique assets identify the targeted subject, one or more medical conditions, or one or more biological responses. In another refinement, the two or more unique assets identify the targeted subject and one or more medical conditions, biological responses, or a combination thereof. For example, the system may generate a unique asset based upon the reference animal data for identification of a subject and generate a unique asset based upon the animal data gathered from the one or more source sensors to positively identify the targeted subject, as well as generate a unique asset based upon the reference animal data for a biological response (e.g., activity) and generate a unique asset based upon the animal data gathered from the one or more source sensors to positively identify the activity in which the targeted subject is engaged in (or was engaged in when the animal data was collected). In this example, multiple identifications can occur utilizing the same animal data derived from the one or more source sensors. In a refinement, two or more identifications (e.g., identifying a targeted individual and their biological response) may be contained within a single unique asset. In some cases, the reference animal data used to create, modify, or enhance the one or more unique assets may be the same or similar. In other cases, different reference animal data may be used to create, modify, or enhance the one or more unique assets. In another refinement, the system may utilize a plurality of unique assets to identify the targeted subject, the one or more medical conditions, or the one more biological responses. The combination of unique assets may more accurately/precisely identify the targeted subject, the one or more medical conditions, or the one or more biological responses.

In another refinement, the system can be operable to enable multiple identifications to occur at the same time. For example, the system may identify the targeted subject and the activity the targeted subject is engaged in. In another example, the system may identify the age of the targeted subject and an associated medical condition. The term “at the same time” can be synonymous with “simultaneous.” In another refinement, “at the same time” can include two or more actions taken concurrently to make two or more identifications that produce results at different times. In another refinement, “at the same time” can also include two or more actions not occurring concurrently so long as the two or more actions are delayed only for the necessary processing required by the one or more computing devices for the multiple identifications. In another refinement, identification occurs in succession or asynchronous. For example, the identification of the subject may take place in the initial stages of the data collection period while the identification of the activity in which the targeted subject is (or was) engaged in or an injury the targeted subjected experienced may occur once the data collection period concludes. In another refinement, one or more identifications can occur in real time or near real-time. In this context, near real-time means that the transmission is not purposely delayed except for necessary processing by the sensor and any other computing device.

In another refinement, two or more unique assets may be created that enable one or more targeted individuals, medical conditions, or biological responses to be identified in two or more ways. For example, the system may create a unique asset (e.g., a unique biological-based digital signature) based on the subject's daily routine using a variety of sensor-based animal data by analyzing how the subject's body responds over a period of time and in a variety of contexts and conditions, and create one or more unique assets that enable identification of the individual in each of the contexts and conditions. In another example, the system may also create a unique asset for the same subject based upon a combination of unique patterns identified in their animal data (e.g., ECG traces, body temperature, and breathing rate) based upon different contextual information (e.g., after waking up, after exercising, after consuming multiple substances such as cups of coffee). In another example, the system may create a unique asset for the same subject based upon their one or more step patterns (e.g., walking, running) and gait analysis, enabling identification of the individual in combination with their one or more attributes (e.g., hair color, body shape). In yet another example, the system may also create a unique asset based upon raw data collected from the same subject over a period of time and use the raw data and its derived metrics with one or more other variables (e.g., activity data, time of day) to create the unique asset (e.g., unique identifier) and identify the individual.

In another variation, identification can include matching the identity of the targeted individual, medical condition, or biological response with a targeted individual, medical condition, or biological response in the reference animal database. Identification occurs based upon an evaluation (e.g., comparison) of the at least one unique asset derived from the targeted subject's sensor-based animal data and at least one unique asset derived from the reference animal data. In a refinement, (1) the collection of animal data by the system from the one or more source sensors, (2) the creation, modification, or enhancement of the at least one unique asset derived from the reference animal data, (3) the comparison of the at least one created, modified, or enhanced unique asset with the animal data derived from the one or more source sensors, or its one or more derivatives, (4) the identification of the targeted subject, the targeted medical condition, or the targeted biological response, or (5) a combination thereof, occurs in real-time or near real-time. In another refinement, an identification (e.g., a match) is characterized by (e.g., includes) at least one of: a percentage match, possibility, probability, prediction, confidence indicator, score, or likelihood. A match can be a partial match (e.g., 90% match, 50% match, 10% match) or an absolute match (i.e., 100% match). In another refinement, a match can be no match (0% match).

In a variation, upon identification of the targeted subject, targeted medical condition, or targeted biological response by the one or more computing devices, the one or more computing devices make one or more verifications. In this context and other similar contexts, verify includes authenticates, validates, confirms, and the like. In a refinement, the one or more computing devices verify the identity of the targeted individual, the targeted medical condition, or the targeted biological response. In another refinement, the one or more computing devices verify the association between the targeted individual and the one or more source sensors. In this context, “association” means that the system confirms that the one or more source sensors are, in fact, collecting animal data from the targeted individual, and the animal data is, in fact, derived from the targeted individual (e.g., the system has correctly assigned the one or more source sensors to the correct targeted individual). While “in fact” can mean absolute (e.g., 100% certainty), in the context of this application, it can also include a likelihood (e.g., 85% likelihood, so the system verifies the association), probability, possibility, prediction, and the like. The verification threshold can be a tunable parameter. In another refinement, the one or more computing devices verify that the one or more source sensors are collecting data from the identified targeted individual. In another refinement, the process of identification includes verification and vice versa.

In another refinement, the one or more computing devices verify the association between the targeted individual and the animal data from the one or more source sensors. In one variation, upon verification, at least a portion of the animal data from the verified subject is distributed by the one or more computing devices to one or more other computing devices for consideration. In another variation, at least a portion of the identified and/or verified animal data or its one or more derivatives is distributed by the one or more computing devices to one or more other computing devices for consideration. In another variation, at least a portion of the identified and/or verified animal data (e.g., including its one or more derivatives) is distributed to one or more computing devices for consideration. In another variation, at least a portion of the animal data from the identified targeted subject or its one or more derivatives is distributed by the one or more computing devices to one or more other computing devices for consideration. In another variation, the animal data from the verified subject is distributed as part of an animal data monetization system whereby the at least a portion of the animal data is distributed to one or more computing devices for consideration (e.g., an animal data marketplace or exchange where acquirers and sellers of animal data or simulated data derived from animal data can participate in a consideration exchange; an animal data-based collateral system; a system which utilizes animal data as a form of digital currency to acquire goods, services, and/or other consideration). Additional details related to a systems and methods for monetizing animal data, including systems that utilize animal data as collateral or as consideration to acquire other consideration, are disclosed in U.S. patent Ser. No. 16/977,454 filed Nov. 5, 2020, and U.S. patent Ser. No. 16/242,708 filed Sep. 10, 2021; the entire disclosures of which is hereby incorporated by reference.

In another variation, animal data-based identification and recognition system 10 can be implemented as part of an animal data-based consideration system (e.g., animal data monetization system, animal data-based collateral system, or digital currency system that utilizes animal data as a form of currency to acquire other consideration). For example, if a third-party acquirer wants to acquire n number of sets of sensor-based animal data from one or more targeted subjects featuring one or more sensor and subject characteristics (e.g., specific age, weight, height, and the like) for consideration, the system can provide verification for the origin of the animal data (e.g., verifying that the animal data collected by the system is, in fact, coming from the desired one or more targeted subjects and from the desired sensor featuring the desired characteristics prior to acquiring the animal data). In this example, the system is implemented for the purpose of verifying the identity of the individual from which the sensor data is collected in order to distribute the data to one or more third parties for consideration. In a variation, the identified and/or verified animal data is distributed as part of an animal data consideration system (e.g., animal data marketplace where data providers such as individuals that generate animal data from one or more sensors can provide data for acquisition and data acquirers can acquire animal data for consideration; a system whereby animal data is used as collateral to obtain consideration such as a loan; a system whereby animal data is used as a form of currency to obtain consideration; and the like). In a refinement, the system verifies the one or more medical conditions of the one or more targeted subjects as part of a consideration system. In another refinement, the system verifies the one or more biological responses of the one or more targeted subjects as part of a consideration system. In another refinement, the system can verify the one or more characteristics related to the one or more source sensors, with the one or more characteristics of the one or more source sensors including at least one of: identity of the sensor, sensor type, sensing type, sensor model, sensor brand, firmware information, sensor positioning, operating parameters, sensor properties, sensor settings, sampling rate, data range, mode of operation, or gain. In a further refinement, verification of the one or more characteristics related to the one or more sensors can occur using at least one unique asset. In another refinement, once the animal data is verified (or validated) and included as part of the reference animal data, one or more searchable tags are created related to the targeted subject, medical condition, biological response, or a combination thereof. In another refinement, the system can verify at least a portion of the metadata (e.g., verify the activity in which the data was collected, time, location, targeted subject attributes, and the like). In another refinement, the system can verify the association between the metadata and the animal data collected via the one or more source sensors. In another refinement, the system can verify one or more characteristics related to the animal data (e.g., duration of data collection period, quality of data, size/volume of the data set, data format, algorithms used to derive or clean data if any, and the like).

In another refinement, a plurality of verifications occur based upon new animal data entering the system via the one or more computing devices. For example, the system may verify that the animal data is being gathered from the identified targeted individual at multiple times during the course of one or more data collection periods (e.g., once identified as the targeted subject, the system will want to re-verify that the data being collected is still being obtained from the targeted subject; once the activity is identified, the system will want to re-verify that the data being collected is still being obtained from the targeted activity; once it is identified that a targeted subject has exhibited a particular biological response—e.g., like flow state or reduced stress, the system will want to re-verify that the data being collected is still being obtained from the targeted subject exhibiting that biological response; once a medical condition is identified, the system will want to re-verify the status of the medical condition via the animal data over a period of time; once an injury is identified, the system will want to re-verify the status of the injury via the animal data over a period of time). The number of times one or more verifications can occur during a data collection period or across multiple data collection periods can be a tunable parameter, meaning a verification can occur every second, minute, hour, day, week, and the like). In the event of re-verification, the system may create one or more new unique assets for each verification, or modify (e.g., update) or modify/enhance one or more existing unique assets based on the new animal data for each verification. In a refinement, a verification occurs upon comparing at least two unique assets, at least one of which is derived from the animal data gathered from the one or more source sensors, to identify the targeted subject, the one or more targeted medical conditions, or the one or more targeted biological responses. In another refinement, the one or more computing devices generate one or more alerts based upon the one or more identifications or verifications. For example, an alert may be generated if a medical condition is detected, or the identity of a subject is identified or verified, or if a biological response is exhibited or achieved. The one or more alerts may be provided to the user via the display, or to another one or more computing devices.

In a refinement, upon identification of the targeted subject, targeted medical condition, or targeted biological response by the one or more computing devices, the one or more computing devices create, modify, assign, or a combination thereof, one or more tags. The one or more tags may also be created modified, assigned, or a combination thereof, for one or more verifications. For example, if the system identifies a new medical condition associated with the targeted subject or a biological response for the targeted subject, or confirms the identity of the targeted subject, or verifies one or more sensor characteristics/parameters, one or more new tags may be created and assigned to the targeted subject (e.g., if a targeted subject is identified to have a respiratory disease, that respiratory disease will be associated with the targeted individual via one or more tags). In a variation, the one or more tags are included as part of the animal data's metadata. In another refinement, the one or more tags are created, modified, assigned, removed, or a combination thereof, based on new data or other information entering the system. For example, if a targeted subject no longer has the respiratory illness, the tag may be modified to reflect the new information.

In a variation, one or more tags can be created based upon one or more characteristics related to the animal data (e.g., including contextual information and other metadata), the one or more targeted subjects (e.g., including their one or more medical conditions or biological responses), the one or more sensors, or a combination thereof. In a refinement, one or more tags can also be created based upon one or more characteristics related to the one or more medical conditions or biological responses. Tags (e.g., including classifications or groups that a targeted subject may be assigned to such as basketball team, individuals with a specific type of disease or blood type, and the like, or classifications or groups that medical conditions or biological responses may be assigned to) can be identifiers for data, can support the indexing and search process for one or more computing devices or data acquirers (e.g., tags can simplify the search process as one or more searchable tags), and may be based on data collection processes, practices, quality, or associations, as well as targeted individual characteristics. A characteristic may include specific personal attributes or characteristics of the one or more subjects or groups of subjects from which the animal data is derived (e.g., name, weight, height, corresponding identification or reference number, medical history, personal history, health history, medical condition, biological response, and the like), as well as information related to the animal data, its associated metadata, and the one or more sources of the animal data such as sensor type, sensor model, sensor brand, firmware information, sensor positioning, time stamps, sensor properties, classifications, specific sensor configurations, operating parameters (e.g., sampling rate, mode, gain, sensing type), mode of operation, data range, location, data format, type of data, algorithms used, quality of the data, size/volume/quantity of the data, analytics applied to the animal data, data value (e.g., actual, perceived, future, expected), when the data was collected, associated organization, associated activity, associated event (e.g., simulated, real world), latency information (e.g., speed at which the data is provided), environmental condition (e.g. if the data was collected in a dangerous condition/environment, rare or desired condition/environment, and the like), bodily condition (e.g., if a person has stage 4 pancreatic cancer or other bodily condition), context (e.g., data includes a monumental moment/occasion, such as achievement of a threshold or milestone within the data collection period may make the data more valuable), duration of data collection period, quality of data (e.g., a rating or other indices applied to the data, completeness of a data set, noise levels within a data set, data format), monetary considerations (e.g., cost to create or acquire, clean, and/or structure the animal data; value assigned to the data), non-monetary considerations (e.g., how much effort and time it took to create or acquire the data), and the like. It should be appreciated that any single characteristic related to animal data (e.g., including any characteristic related to the data, the one or more sensors, the metadata, the one or more targeted subjects, the one or more medical conditions, the one or more biological responses, and the like) can be assigned or associated with one or more tags. Characteristically, the one or more tags associated with the animal data can contribute to creating, modifying, or enhancing an associated value (e.g., monetary, non-monetary) for the animal data. In a refinement, one or more artificial intelligence techniques (e.g., machine learning, one or more neural networks) are utilized to assign, create, modify, remove, or a combination thereof, one or more tags related to the animal data (e.g., including its metadata), the one or more targeted subjects, the one or more source sensors, the one or more medical conditions, the one or more biological responses, or a combination thereof. In another refinement, the one or more computing devices verify the one or more tags associated with the targeted individual, the one or more source sensors, the animal data (e.g., including its metadata), the one or more medical conditions, the one or more biological responses, or a combination thereof.

Upon identification of the targeted subject, targeted medical condition, or targeted biological response by the system, the system can associate at least a portion of the animal data derived from the one or more source sensors, or its one or more derivatives, with the targeted subject, the targeted medical condition, or the targeted biological response. In another variation, the system can associate at least a portion of the animal data derived from the one or more source sensors, or its one or more derivatives, with the targeted subject, the targeted medical condition, or the targeted biological response after one or more verifications.

In a refinement, the comparison between the at least one created, modified, or enhanced unique asset and the animal data derived from the one or more source sensors, or its one or more derivatives, verifies the origin of the animal data derived from the one or more source sensors, or its one or more derivatives. In a variation, the origin can be the targeted subject. In another variation, the origin can be the one or more source sensors. In another variation, the origin can be one or more characteristics/attributes related to the animal data (e.g., the metadata associated with the animal data). In another variation, the origin can be a combination thereof.

In another refinement, the one or more computing devices are operable to assign (e.g., associate), and/or verify the assignment of, the gathered animal data from the one or more source sensors to the targeted individual. For example, upon verification that the animal data being collected from the one or more source sensors are in fact derived from the targeted individual, the system can verify that the sensor being utilized is correctly associated with the targeted individual in the system.

In another refinement, the one or more source sensors provide at least one characteristic related to the one or more source sensors prior to identifying or verifying the targeted individual, medical condition, or biological response, the at least one characteristic being provided from a group consisting of: identity of the sensor, sensor type, sensing type, sensor brand, sensor model, firmware information, sensor positioning, operating parameters, sensor properties, sensor settings, sampling rate, mode of operation, battery life, data range, or gain. In another refinement, one or more evaluations are made using at least one characteristic related to the one or more source sensors or the gathered animal data prior to identifying or verifying the targeted individual, medical condition, or biological response, the at least one characteristic being provided from a group consisting of: identity of the sensor, sensor type, sensing type, sensor brand, sensor model, sensor firmware information, sensor positioning, sensor operating parameters, sensor properties, sensor settings, sensor sampling rate, sensor mode of operation, sensor gain, data range, sensor battery life, time stamps, location, data format, type of data, algorithms used, quality of data, size/volume/quantity of the data, latency information, environmental condition, bodily condition, context related to the data collected (e.g., activity), duration of data collection period, quality of data, or when the data was collected. In another refinement, the at least one unique asset can be utilized to identify at least one sensor parameter, with the at least one sensor parameter being provided from a group consisting of: identity of the sensor, sensor type, sensing type, sensor brand, sensor model, firmware information, sensor positioning, operating parameters, sensor properties, sensor settings, sampling rate, mode of operation, battery life, data range, or gain.

In many variations, at least one of the one or more source sensors is a biosensor that gathers physiological, biometric, chemical, biomechanical, location, environmental, genetic, genomic, glycomic, or other biological data from one or more targeted individuals. In a refinement, the one or more biosensors gathers, or provides information that can be converted into, at least one of the following types of animal data: facial recognition data, eye tracking & recognition data, blood flow data, blood volume data, blood pressure data, biological fluid data, body composition data, biochemical data, pulse data, oxygenation data, core body temperature data, skin temperature data, galvanic skin response data, perspiration data, location data, positional data, audio data, biomechanical data, hydration data, heart-based data, neurological data, genetic data, genomic data, skeletal data, muscle data, respiratory data, kinesthetic data, ear acoustic authentication data, finger vein recognition data, fingerprint recognition data, footprint and foot dynamics data, hand geometry data, body odor recognition data, palm print recognition data, palm vein recognition data, skin reflection data, thermography recognition data, keystroke dynamics data (e.g., including usage patterns on computing devices such as mobile phones), signature recognition data, speaker recognition data, voice recognition data, gait recognition data, or lip motion data.

In a refinement, the at least one unique asset is derived from at least a portion of animal data gathered from the one or more biosensors. “Gathered from” is inclusive of “provided by,” meaning animal data that is gathered from the one or more biosensors can also be animal data that is provided by the one or more biosensors. In another refinement, the at least one unique asset is derived from two or more types of animal data gathered from the one or more biosensors. In a further refinement, the at least one unique asset is derived from animal data gathered from two or more biosensors. In a further refinement, the at least one unique asset includes at least a portion of non-animal data. In a further refinement, the at least one unique asset incorporates at least one of or any combination of: name, age, weight, height, eye color, skin color, hair color (if any), birthdate, race, reference identification (e.g., social security number, national ID number, digital identification) country of origin, area of origin, ethnicity, current residence, addresses, phone number, gender of the targeted individual from which the animal data originated, data quality assessment, information gathered from medication history, medical history, medical records, health records, genetic-derived data, genomic-derived data, medical conditions, traits, health risks, inherited conditions, drug responses, DNA sequences, protein sequences and structures, biological fluid-derived data, drug/prescription records, allergies, family history, health history (including mental health history), blood analysis, physical shape, manually-inputted personal data, historical personal data, the one or more activities the targeted individual is engaged in while the animal data is collected, ambient temperature data related to the animal data, humidity data related to the animal data, barometric pressure data related to the animal data, elevation data related to the animal data, one or more associated groups, one or more nutritional habits, one or more activity habits, one or more health habits, one or more technology habits, one or more social habits (e.g., tobacco use, alcohol consumption, exercise habits, nutritional diet, and the like), education records, criminal records, financial information (e.g., bank records, such as bank account instructions, checking account numbers, savings account numbers, credit score, net worth, transactional data), social data (e.g., social media accounts, social media history, records, internet search data, social media profiles, metaverse profiles, metaverse activities/history), employment history, marital history, relatives or kin history (in the case the targeted subject has one or more children, parents, siblings, and the like), relatives or kin medical history, relatives or kin health history, manually-inputted personal data (e.g., one or more locations where a targeted individual has lived, emotional feelings, mental health data, preferences), historical personal data, or individual-generated data.

In a refinement, the one or more computing devices dynamically create, modify, or enhance the at least one unique asset based upon the animal data collected by the one or more source sensors. For example, the system may recognize that only specific types of animal data are being collected by the one or more source sensors for a specific individual. In this case, the system may generate the at least one unique asset from the reference animal data utilizing only the one or more types of animal data being collected via the one or more source sensors while generating another at least one unique asset for the individual based upon their collected animal data from the one or more source sensors in order to identify the targeted subject, the medical condition, or the biological response. In a variation, the system dynamically creates, modifies, or enhances the at least one unique asset based upon the available animal data derived from the one or more source sensors. In another refinement, the one or more computing devices dynamically create, modify, or enhance the at least one unique asset based upon new animal data entering the system. In another refinement, the one or more computing devices dynamically create, modify, or enhance the at least one unique asset based upon new reference animal data collected by the system. In a refinement, the one or more computing devices dynamically create, modify, or enhance the at least one unique asset based upon the available metadata associated with the animal data (e.g., one or more characteristics related to the one or more source sensors, one or more variables that may have impacted the animal data being collected, and the like).

In a refinement, the at least one unique asset or the one or more derivatives from the animal data are created, modified, or enhanced upon the one or more computing devices gathering animal data, reference animal data, or both. For example, as new animal data—including reference animal data and associated metadata—enters the system, the at least one unique asset may be created, modified, or enhanced. In another refinement, the comparison between the at least one unique asset and the gathered animal data or its one or more derivatives occurs once, intermittently, or regularly to verify the targeted individual, the targeted medical condition, or the targeted biological response (e.g., every second, every minute, every hour, every day, and the like). The frequency of the one or more verifications is a tunable parameter.

In a refinement, the comparison between the at least one unique asset (e.g., derived from reference animal data) and the gathered animal data or its one or more derivatives identifies multiple medical conditions or biological responses. In another refinement, the comparison between the at least one unique asset and the gathered animal data or its one or more derivatives identifies multiple targeted subjects. In another refinement, the at least one unique asset can be utilized to identify or verify a single targeted subject, medical condition, or biological response. In another refinement, the at least one unique asset can be utilized to identify or verify multiple targeted subjects, medical conditions, or biological responses.

In a refinement, the at least one unique asset is created, modified, or enhanced using two or more types of animal data, at least one of which is physiological data. In another refinement, the at least one unique asset is created, modified, or enhanced using two or more types of animal data, at least one of which is biological fluid data. In another refinement, the at least one unique asset is created, modified, or enhanced using two or more types of animal data, at least one of which is biomechanical data. In another refinement, the at least one unique asset is created, modified, or enhanced using two or more types of animal data, at least one of which is genomic-based data. In another refinement, the at least one unique asset is created, modified, or enhanced using two or more types of animal data, at least one of which is genetic-based data. In another refinement, the at least one unique asset is created, modified, or enhanced using two or more types of animal data, at least one of which is location-based data. In another refinement, the at least one unique asset is created, modified, or enhanced using two or more types of animal data, at least one of which is chemical-based data. In another refinement, the at least one unique asset is created, modified, or enhanced using two or more types of animal data, at least one of which is biochemical-based data. In another refinement, the at least one unique asset is created, modified, or enhanced using two or more types of animal data, at least one of which is biometric data.

In a refinement, the comparison between the at least one unique asset (e.g., derived from reference animal data) and the gathered animal data or its one or more derivatives invalidates (e.g., dismisses, nullifies) the association (e.g., including the presumed association) of the targeted subject, targeted medical condition, or targeted biological response with the animal data derived from the one or more source sensors and/or the one or more sensors. In another refinement, the comparison (e.g., evaluation, analysis) between the at least one unique asset and the gathered animal data or its one or more derivatives invalidates the identification of the targeted subject, targeted medical condition, or targeted biological response. In a variation, the comparison between the at least one unique asset and the gathered animal data or its one or more derivatives results in a non-identification or negative identification of the targeted subject, targeted medical condition, or targeted biological response (e.g., “no match found”; excluding individuals, conditions, or responses from search results). In another refinement, identification of the targeted subject, targeted medical condition, or targeted biological response includes not recognizing the targeted subject, targeted medical condition, or targeted biological response. In another refinement, the comparison between the two or more unique assets identifies the targeted subject as two or more subjects (e.g., two or more known subjects).

In a refinement, the animal data derived from one or more source sensors is collected across multiple activities, from which one or more unique assets are derived. For example, a unique, repeatable pattern in the animal data may be derived for an individual using the same type of animal data or multiple types of animal data across multiple activities (e.g., sitting and standing up while responding to a visual, audio, or physical stimuli using only ECG data). The system may store this information as part of the one or more digital records in the reference animal database associated with that individual, and access the one or more digital records to generate at least one unique asset in order to identify and/or verify the individual based upon the patterns exhibited in their animal data from the one or more source sensors (e.g., which are transformed into a unique asset to enable identification and/or verification). In another refinement, one or more changes or variations in the same type of animal data collected by the one or more source sensors occur based upon one or more variables, enabling the system to generate one or more unique assets from which one or more identifications and/or verifications occur.

Characteristically, one or more artificial intelligence techniques (e.g., machine learning techniques, deep learning techniques) can be utilized to create, modify, or enhance one or more unique assets. One or more artificial intelligence techniques can also be utilized to compare the gathered animal data from the one or more source sensors or its one or more derivatives with (e.g., or against) the one or more unique assets by the one or more computing devices to identify the targeted subject, the medical condition, or biological response. In some cases, the use of one or more artificial intelligence techniques enables the AI to create a picture of the subject's body and its associated biological functions based upon animal data (e.g., create a digital map of biological functions associated with contextual data and other data that is unique and specific to an individual or a subset of individuals) in order to create a unique asset based upon that data. For example, by utilizing one or more artificial intelligence techniques, the system can analyze both reference animal data and current animal data from the one or more sensors to create, modify, or enhance one or more unique assets that identify the targeted subject, one or more medical conditions, or one or more biological responses. Given that machine learning and deep learning-based systems are set up to learn from collected data rather than require explicit programmed instructions, its ability to search for and recognize patterns that may be hidden within the reference animal data and the gathered sensor data from the one or more source sensors enable machine learning and other AI-based systems to uncover insights from collected data that allow unique assets (e.g., unique biological-based identifiers) to be uncovered for each individual based upon their animal data. Advantageously, because machine learning and deep learning-based systems use data to learn, it oftentimes takes an iterative approach to improve model prediction and accuracy as new data or preferences enter the system, as well as improvements to model prediction and accuracy derived from feedback provided from previous computations made by the system (which also enables production of reliable results). In such a scenario, new animal data from the one or more source sensors or new reference animal data entering the system at any given time enables a new, deeper understanding of the individual based upon a broader set of data.

By utilizing one or more artificial intelligence techniques such as machine learning or deep learning techniques, the system can identify one or more patterns in the reference animal data that make each individual data set unique or identifiable when compared to the other one or more reference animal data sets (thereby making each individual unique). With each individual having at least one unique asset (e.g., at least one unique biological-based identifier) based upon the reference animal data, the system can analyze the incoming sensor-based animal data (e.g., in conjunction with the one or more variables and other metadata, which may include other animal and/or non-animal data) to identify one or more unique characteristics within the targeted individual's animal data (e.g., one or more unique biological characteristics, which—either alone or in combination—can create one or more unique biological patterns or signatures or the like specific to that individual) to derive the one or more unique assets that identify the targeted individual. Advantageously, depending on the data being collected by the system from the one or more source sensors, the system may be operable to identify the one or more types of animal data currently being collected from the targeted subject by the system via the one or more source sensors, and create a unique identifier only based on the data currently being collected by the one or more source sensors, thus identifying the targeted subject, medical condition, or biological response based upon their available data. In a variation, the one or more computing devices create, modify, or enhance the at least one unique asset from animal data that is available as both reference animal data and animal data gathered by the one or more source sensors from the targeted subject. For example, if an ECG-based sensor is not being used by the targeted subject, then the system will not create a unique signature utilizing ECG-based data. In a variation, the one or more computing devices selectively use a subset of the one or more unique assets from the reference animal data such that the subset can be compared against the animal data that can be captured by the one or more source sensors from the targeted subject. In another variation, the one or more computing devices selectively use a subset of animal data from the reference animal data such that the subset can be compared against the animal data that can be captured by the one or more source sensors from the targeted subject.

In a refinement, the creation, modification, or enhancement of the animal data or its one or more derivatives (e.g., one or more unique assets) utilizes at least a portion of artificial data. Artificial data can be derived from one or more simulated events, concepts, objects, or systems, and can be generated using one or more statistical models or artificial intelligence techniques. In a variation, artificial data can be used to assess one or more biological-based occurrences of participants in a simulation, with the simulation being operable to enable the modification of one or more variables in order to generate simulated data with desired conditions (e.g., generating a specific type of animal data when the individual is participating in a specific activity in specific environmental conditions with specific medical conditions associated with the individual). Advantageously, artificial data can be used to predict one or more future biological outcomes for any given targeted individual based upon one or more characteristics related to the targeted individual, the one or more sensors, or the animal data (e.g., including other metadata such as the activity in which the animal data was collected). In this regard, the artificial data can be utilized as a baseline for any given individual, medical condition, or biological response to compare current animal data readings (and unique assets derived from it) with predicted readings. Artificial data may be incorporated as part of the reference animal data to derive the at least one unique asset, and/or as part of the one or more animal data sets gathered from the one or more source sensors to derive the at least one unique asset.

In another refinement, the at least one unique asset (e.g., biological signature) is created, modified, or enhanced using one or more artificial intelligence techniques based upon a subject's one or more biological patterns from one or more types of animal data. In this refinement, the system can leverage the one or more artificial intelligence techniques to enhance or predict what the subject's body will do in one or more modeled scenarios and create, modify, or enhance one or more unique assets in order to compare existing animal data with the subject's future animal data at any given point in time. For example, as a subject ages, the system can run one or more simulations to predict what the subject's one or more animal data readings should look like, and create one or more unique assets that enable the system to create comparisons with the subject at any moment in time. In another example, if the subject is traveling to a specific destination for a period of time, the system can model the subject's exposure to one or more environmental conditions (e.g., air pollution) or other conditions and predict one or more outcomes (e.g., lung or respiration issues) that will enable a more tailored baseline for the creation, modification, or enhancement of the one or more unique assets generated during and after that period of time, allowing for more accurate identification of the targeted subject, medical condition, or biological response.

In a refinement, the system makes one or more one or more identifications and/or verifications utilizing at least a portion of artificial animal data. In this scenario, the system may make one or more predictions related to what the individual's body will do in a future state in order to make the one or more identifications and/or verifications. For example, the system may only have ECG data from an individual that is outdated (e.g., the ECG data may be 3-5 years old). The system can utilize one or more artificial intelligence techniques to look at other ECG datasets in the reference animal database to learn how ECG patterns can change based upon a 3-5 year age increase while taking into account one or more characteristics of the individual (e.g., age, weight, associated medical conditions, health history, and the like). The system can then generate artificial animal data to predict what the individual's ECG will look like in a future state (e.g., today compared to 3-5 years ago), and utilize that artificial animal data to make one or more identifications and/or verifications utilizing the artificial animal data and the animal data being gathered by the system via the one or more source sensors. In another refinement, if the system gathers animal data from one or more source sensors that is not included in the reference animal data, the system may generate artificial animal data for one or more reference individuals to enable the creation, modification, or enhancement of the at least one unique asset.

In another refinement, the at least one unique asset or derivative of the animal data is created, modified, or enhanced utilizing one or more artificial intelligence techniques via the use of one or more neural networks. In general, a neural network can support the system with a variety of pattern recognition-based tasks (e.g., support in the identification, creation, modification, and/or enhancement of one or more unique assets) and other described functions that require a relational understanding of gathered data (e.g., animal data, non-animal data, reference animal data, and the like) to support the one or more identifications and/or verifications, as well as support the system in generating artificial animal data after being trained with real animal data. In the case of artificial data creation, animal data (e.g., ECG signals, heart rate, biological fluid readings) is collected from one or more sensors from one or more target individuals typically as a time series of observations. Sequence prediction machine learning algorithms can be applied to predict possible animal data values based on collected data. The collected animal data values will be passed on to one or more models during the training phase of the neural network. The neural network utilized to model the non-linear data set (or in some variations, linear data set) will train itself based on established principles of the one or more neural networks. In another refinement, the one or more artificial intelligence techniques includes execution of one or more trained neural networks. In another refinement, the one or more trained neural networks utilized to generate the at least one unique asset and/or support one or more system functions that enable one or more identifications and/or verifications consists of one or more of the following types of neural networks: Feedforward, Perceptron, Deep Feedforward, Radial Basis Network, Gated Recurrent Unit, Autoencoder (AE), Variational AE, Denoising AE, Sparse AE, Markov Chain, Hopfield Network, Boltzmann Machine, Restricted BM, Deep Belief Network, Deep Convolutional Network, Deconvolutional Network, Deep Convolutional Inverse Graphics Network, Liquid State Machine, Extreme Learning Machine, Echo State Network, Deep Residual Network, Kohenen Network, Support Vector Machine, Neural Turing Machine, Group Method of Data Handling, Probabilistic, Time delay, Convolutional, Deep Stacking Network, General Regression Neural Network, Self-Organizing Map, Learning Vector Quantization, Simple Recurrent, Reservoir Computing, Echo State, Bi-Directional, Hierarchal, Stochastic, Genetic Scale, Modular, Committee of Machines, Associative, Physical, Instantaneously Trained, Spiking, Regulatory Feedback, Neocognitron, Compound Hierarchical-Deep Models, Deep Predictive Coding Network, Multilayer Kernel Machine, Dynamic, Cascading, Neuro-Fuzzy, Compositional Pattern-Producing, Memory Networks, One-shot Associative Memory, Hierarchical Temporal Memory, Holographic Associative Memory, Semantic Hashing, Pointer Networks, Encoder-Decoder Network, Recurrent Neural Network, Long Short-Term Memory Recurrent Neural Network, or Generative Adversarial Network.

In a variation, the creation (e.g., formulation) of the one or more unique assets is directed by the one or more computing devices that store or have access to the reference animal data based upon one or more artificial intelligence techniques. For example, the one or more computing devices creating, modifying, or enhancing the one or more unique assets with the reference animal data may already have one or more unique assets created for each individual based on pre-established patterns or a pre-determined framework implemented by the system that can create one or more unique assets across multiple subjects. The system may then direct the user via the display device as to the type of animal data required to be collected via the one or more source sensors or computing devices, or may specify which one of the one or more sensors are the one or more source sensors to be utilized, to make the one or more identifications or verifications. In a refinement, the system may also specify the one or more variables required to create, modify, or enhance the unique asset and/or make one or more identifications and/or verifications (e.g., contextual data such as duration of collection, activity in which the data is being collected, and the like). Based upon the source sensor data entering the system and corresponding available information (e.g., contextual data, other metadata), the one or more computing devices can provide the relevant reference animal-based unique asset (e.g., unique identifier) based upon the source sensor data and available information to identify the individual. In another variation, the formulation of the one or more unique assets is directed by the one or more computing devices that are collecting the animal data from the one or more source sensors based upon one or more artificial intelligence techniques. For example, the system collecting the data from the one or more source sensors may only be collecting certain types of animal data and have available only certain animal data and non-animal data information available. In this scenario, the computing device may inform the system creating the one or more unique assets with the reference animal data that the one or more unique assets need to be created, modified, or enhanced based on the specific type(s) of animal data available and its corresponding available information (e.g., contextual data).

In another refinement, the system is operable to detect one or more outlier or missing data values (e.g., including signals) from the gathered animal data (e.g., the reference animal data, the animal data generated from one or more sensors, or both) and generate one or more artificial data values to replace the one or more outlier or missing values in order to enable identification of one or more subjects, medical conditions, or biological responses. In many cases, the one or more sensors produce measurements that are provided to a server, with the sensor or server applying methods or techniques to filter the data and generate one or more animal data values (e.g., ECG values, heart rate values). However, in cases where data has an extremely low signal-to-noise ratio, or in some cases when one or more values are missing, or in other cases where the sensor is not able to derive consistent data (e.g., due to incorrect placement of the sensor, activity that produces “bad” data), pre-filter logic may be required to generate artificial data values. In one aspect, a pre-filter method whereby the system takes a number of steps to “fix” the data generated from one or more sensors to ensure that the one or more data values generated are clean and fit within a predetermined range may be utilized. The pre-filter logic would ingest the data from the sensor, detect any outlier or “bad” values, replace these values with expected or “good” artificial values and pass along the “good” artificial values as its computation of the one or more animal data values (e.g., heart rate values). The term “fix” refers to an ability to create one or more alternative data values (i.e., “good” values) to replace values that may fall out of a preestablished threshold, with the one or more “good” data values aligning in the time series of generated values and fitting within a preestablished threshold. Advantageously, the pre-filter logic and methodology for identification and replacement of one or more data values can be applied to any type of sensor data collected, including both raw and processed outputs.

The pre-filter logic becomes important in a scenario whereby the signal-to-noise ratio in the time series of generated values from one or more sensors is at or close to zero, or numerically small. In this case, systems that gather animal data may ignore one or more such values, which in some cases may result in no value generated or a generated value that may fall outside the pre-established parameters, patterns and/or thresholds. Such values may result from the subject taking an action that is not optimal based upon the sensor (e.g., significant motion or movement for an ECG sensor when little to no movement is required), or in competing signals derived from the same sensor being introduced or deteriorating the connection, or from other variables. This in turn may make for an inconsistent animal data series. To solve for this problem, a method whereby one or more data values are created by looking at future values rather than previously generated values can be established. More specifically, the system may detect one or more outlier signal values and replace outlier values with one or more signal values that fall within an expected range (e.g., the established upper and lower bounds), thus having the effect of smoothing the series while at the same time decreasing the variance between each value. The established expected range may take into account a number of different variables including the individual, the type of sensor, one or more sensor parameters, one or more of the sensor characteristics, one or more variables (e.g., environmental factors), one or more characteristics of the individual, activity of the individual, and the like. The expected range may also be created by one or more artificial intelligence techniques that uses at least a portion of previously collected sensor data or one or more derivatives, as well as one or more variables, to predict what an expected range may be. The expected range may also change over a period of time and be dynamic in nature, adjusting based on one or more variables (e.g., the activity the person is engaged in or environmental conditions). In a variation, one or more artificial intelligence techniques may be utilized, at least in part, to generate one or more artificial signal values within the expected range (e.g., upper and lower bound) derived from at least a portion of gathered animal data from the one or more sensors, or one or more derivatives.

In a variation, one or more unique assets may be created, modified, or derived from animal data (e.g., raw animal data) that enables multiple types of animal data (e.g., computed assets) to be derived and used as part of the unique asset. For example, a sensor may collect raw data from which ECG data related to a targeted subject is derived. The ECG data may further provide heart rate data from the targeted subject. The system may identify one or more patterns or trends in the raw data, the ECG data, and the heart rate, combine the patterns or trends and associate the pattens or trends with one or more animal data-based attributes related to the targeted subject (e.g., age, weight, height, medical history, health habits, and the like), one or more types of other animal data information (e.g., activity) and non-animal data information (e.g., air temperature), to create, modify, or enhance the one or more unique assets (e.g., unique identifier). In this scenario, the system may notify one or more computing devices that store, or have access to, the reference animal data, or the one or more computing devices that create, modify, or enhance the one or more unique assets, of the one or more types of animal data being collected, the associated contextual data (e.g., the one or more variables), and the specific type of unique asset (e.g., unique biological signature, identifier) that needs to be created, modified, or enhanced in order to be able to identify the targeted subject. In another scenario, the one or more unique assets created, modified, or enhanced from the raw animal data may have been done so based upon a pre-defined digital signature, identifier, rhythm, pattern, trend, measurement, feature, characteristic/attribute, outlier, data set, or anomaly already determined based upon the one or more unique assets created from the reference animal data. The system can be operable to combine any number of animal data types, attributes, and other animal data and non-animal data information to create, modify, or enhance the at least one unique asset.

In a refinement, the at least one unique asset may be combined with animal data or its one or more derivatives, which can include animal data derived from one or more sensors, to identify and/or verify the targeted individual, the one or more medical conditions, or the one or more biological responses. The at least one unique asset may also be combined with non-animal data, which may be derived from one or more sensors, to execute the one or more identifications and/or verifications. For example, the at least one unique asset may identify a targeted individual with n % accuracy (e.g., 75%). However, the system may also capture another one or more types of animal data (e.g., facial recognition data of, or biomechanical data from, the identified subject), non-sensor animal data (e.g., age and weight data to confirm the physical appearance of the subject matches the expected age and appearance of the targeted subject), non-animal data (e.g., optical-based data that identifies the sensor on the body of the subject), or a combination thereof, to more accurately identify and/or verify the targeted individual and the associated one or more sensors.

In another refinement, the at least one unique asset evaluates (e.g., analyzes), or is utilized in conjunction with, at least one of the following types of data to identify and/or verify the targeted individual, the one or more medical conditions, or the one or more biological responses: facial recognition data, eye tracking & recognition data, blood flow data, blood volume data, blood pressure data, biological fluid data, body composition data, biochemical data, pulse data, oxygenation data, core body temperature data, skin temperature data, galvanic skin response data, perspiration data, location data, positional data, audio data, biomechanical data, hydration data, heart-based data, neurological data, genetic data, genomic data, skeletal data, muscle data, respiratory data, kinesthetic data, ear acoustic authentication data, finger vein recognition data, fingerprint recognition data, footprint and foot dynamics data, hand geometry data, body odor recognition data, palm print recognition data, palm vein recognition data, skin reflection data, thermography recognition data, keystroke dynamics data (e.g., including usage patterns on computing devices such as mobile phones), signature recognition data, speaker recognition data, voice recognition data, gait recognition data, or lip motion data, sensor recognition data, age, weight, height, eye color, skin color, hair color (if any), birthdate, race, reference identification, country of origin, area of origin, ethnicity, current residence, addresses, phone number, gender, data quality assessment, information gathered from medication history, medical history, medical records, heath records, health records, genetic-derived data, genomic-derived data, medical conditions, traits, health risks, inherited conditions, drug responses, DNA sequences, protein sequences and structures, biological fluid-derived data, drug/prescription records, allergies, family history, health history, blood analysis, physical shape, manually-inputted personal data, historical personal data, the one or more activities the targeted individual is engaged in while the animal data is collected, ambient temperature data related to the animal data, humidity data related to the animal data, barometric pressure data related to the animal data, elevation data related to the animal data, one or more associated groups, one or more nutritional habits, one or more activity habits, one or more health habits, one or more social habits, education records, criminal records, financial information, social data, employment history, marital history, relatives or kin history, relatives or kin medical history, relatives or kin health history, manually inputted personal data, historical personal data, or individual-generated data.

In another refinement, the system may establish two-way communication with the one or more sensors and communicate directly with the one or more sensors that are capturing animal data from the one or more subjects to initiate one or more actions on the one or more source sensors (e.g., make the sensor create a physical, visual, or audio effect such as vibrate, blink a light or change color, or make a noise) for the purposes of confirming the identity of the one or more source sensors, confirming the one or more source sensors are (in fact) collecting animal data from the targeted individual, or both. Such an action may be verified by one or more other sensors (e.g., optical sensor) that captures the visual confirmation of the one or more sensors collecting data from the targeted subject and/or the one or more actions of the one or more sensors. In another refinement, the system may initiate one or more stimuli originating from one or more sensors that can induce one or more biological phenomena in the animal data of the targeted subject (e.g., changes in their one or more biological readings based upon the one or more initiated stimuli) which enables the creation of one or more unique assets that can identify the targeted subject as the source of the animal data.

In another refinement, the at least one unique asset (e.g., biological signature) is created, modified, or enhanced based on a subject's biological response to one or more controlled stimuli (e.g., physical, visual, auditory), or one or more actions taken by the subject. The system can monitor how the subject's body responds to the one or more controlled stimuli and observe (and record) the biological phenomena that occur based upon the one or more controlled stimuli via the one or more source sensors (e.g., evaluating the one or more biological responses via the one or more source sensors, such as fluctuations or changes in physiological parameters, reaction time, and the like). Characteristically, the one or more controlled stimuli may be implemented (e.g., presented, introduced, enabled) by one or more computing devices (e.g., via one or more displays), by one or more sensors, or by one or more devices associated with the one or more computing devices. The system can create the at least one unique asset by comparing the deviation of the animal data in response to the stimuli or the one or more actions of the subject with the subject's baseline animal data in the same or similar situations without the stimuli or action(s) present. The at least one unique asset can then be utilized to identify the subject. In a variation, the one or more computing devices may introduce—via one or more displays, external hardware devices, sensors, or other sources—one or more variables (e.g., stimuli) as part of the data gathering process to collect animal data via one or more source sensors from the one or more targeted individuals. The targeted individual's biological response to the one or more variables can be utilized, at least in part, to create, modify, or enhance one or more unique assets from which one or more identifications and/or verifications occur.

In a refinement, the comparison between the at least one created, modified, or enhanced unique asset and the animal data derived from the one or more source sensors, or its one or more derivatives, identifies, mitigates, or prevents one or more risks (e.g., including fraudulent behavior). For example, in the context of sports betting (i.e., sports wagering), the comparison between the at least one unique asset and the animal data or its one or more derivatives may identify whether a subject participating in a sports competition is intentionally circumventing, altering, influencing, or inducing one or more biological responses in a given scenario or time period to intentionally influence, induce, elicit, or enable an outcome that is associated with one or more wagers. In many variations, the outcome is a negative outcome (e.g., subject misses a shot, subject loses a match, subject allows for another subject to score, and the like) and the subject's intentional circumvention, influence, or inducement of their one or more biological responses equates to cheating or engaging in fraudulent behavior. Characteristically, the system is configured to identify one or more patterns, trends, features, measurements, outliers, abnormalities, anomalies, readings, characteristics, or the like related to the subject's body (i.e., abnormalities or anomalies in the subject's biological responses) in any given situation (e.g., point in time, environmental conditions) based upon the subject's typical/natural/expected biological responses in that context—sourced from the reference animal data—and the captured animal data from the one or more source sensors (e.g., biological data) and other gathered data (e.g., other variables) that provide context to the one or more patterns, trends, features, measurements, outliers, abnormalities, anomalies, readings, characteristics, or the like in the animal data (e.g., a large bet placed or unusual betting patterns on or related to the one or more subjects in a given situation where the abnormalities or anomalies occur). Additional details related to a system for using animal data in sports betting and other risk mitigation systems are disclosed in U.S. patent Ser. No. 16/977,278 filed Sep. 1, 2020, the entire disclosure of which is hereby incorporated by reference. For example, the system can be configured to identify one or more patterns, trends, features, measurements, outliers, abnormalities, anomalies, readings, characteristics, or the like in the athlete's reference animal data (e.g., what the athlete's body typically does) in any given context/scenario (e.g., based upon contextual data such as number of miles run, the environmental factors, time in the game, score, the outcome that was associated with the animal data, and the like), from which one or more unique assets are created. A unique asset in this scenario can include (1) the athlete's sensor-based animal data readings in light of the context in which the data is collected and the outcomes associated with that data, (2) the one or more patterns, trends, features, measurements, outliers, abnormalities, anomalies, and/or characteristics within the sensor-based animal data readings in the reference animal database (i.e., in light of the context in which the data is collected and the outcomes associated with that data), or (3) a combination thereof. The one or more unique assets—as part of the reference animal data—can be included as part of one or more digital records in the reference animal database associated with the athlete, the data type, and/or other contextual data. The system can be further configured to (1) readily access the reference animal data (e.g., the athlete's reference animal data including their one or more unique assets; other reference animal data that may feature other athletes or data types with one or more similar characteristics to the targeted athlete), (2) collect the live, in-competition animal data from the one or more source sensors (e.g., in a real-time or near real-time setting), and (3) gather metadata from the live competition (e.g., to provide context to the collected animal data) in order to compare reference animal data with the athlete's live, in-competition animal data. The system can be further configured to identify one or more similarities, dissimilarities, or a combination thereof, between the one or more patterns, trends, features, measurements, outliers, anomalies, readings, characteristics, or the like in the reference animal data and the athlete's live, in-competition animal data (e.g., collected via one or more source sensors) in light of other collected information (e.g., contextual data). Characteristically, the system is operable to identify one or more uncharacteristic or unusual (e.g., abnormal) patterns, trends, features, measurements, outliers, anomalies, readings, characteristics, or the like (e.g., deviations or changes in typical biological behavior based on the context; abnormalities in the animal data from the one or more source sensors based upon the context) in the live, in-competition animal data readings based upon one or more comparisons of the in-competition animal data and the reference animal data. The one or more comparisons can occur in real-time or near real-time. Based upon the one or more comparisons, the system can make one or more determinations (e.g., probability, percentage match, possibility, probability, prediction, likelihood, or the like) as to whether the athlete is intentionally influencing, circumventing, altering, or inducing their one or more biological responses in the live competition to intentionally influence, induce, elicit, or enable a specific outcome (e.g., lose one or more games, miss one or more shots) or whether the one or more uncharacteristic or unusual patterns, trends, features, measurements, outliers, anomalies, readings, characteristics, or the like are a result of the context or other information (e.g., the athlete may not need to be exerting the energy required to win the match given the competition; the athlete may be playing an opponent who is superior in skill and therefore the athlete is likely to lose the match, so physiological output may be minimal given the skill of the other opponent; environmental conditions may create conditions where the athlete does not need to exert as much physical energy; the athlete may be having a medical or health issue which can be identified based upon the other source sensor-based animal data readings and a comparison with the reference animal data; and the like). The one or more comparisons may be converted into one or more insights or predictive indicators and utilized as one or more indicators (e.g., integrity indicators) capable of identifying, verifying, assessing, projecting, determining, and/or predicting risk (e.g., fraudulent behavior), or utilized as part of a system related to monitoring and/or maintaining the integrity of a competition and the integrity of the one or more bets associated with the competition (e.g., to prevent competition fraud, wagering fraud, and the like, as well as to identify fraudulent behavior by the one or more individuals involved in the competition). Similarly, the comparison between the at least one created, modified, or enhanced unique asset and the animal data derived from the one or more source sensors, or its one or more derivatives, may be used to determine whether an individual or group of individuals intentionally (e.g., purposefully) change or modify one or more biological responses (e.g., biological-based behaviors or phenomena) to intentionally influence, induce, elicit, or enable an outcome (e.g., purposefully lose a competition such as a sports match, which may be detected based upon comparison of the unique asset and the animal data, or from the animal data itself). In a variation, the comparison between the at least one created, modified, or enhanced unique asset and the animal data derived from the one or more source sensors, or its one or more derivatives, enables one or more insights or predictive indicators to be created, modified, or enhanced based upon the one or more biological responses. In another variation, the system generates and provides one or more alerts to one or more computing devices (e.g., sports wagering systems or other third-party systems) based on the one or more identifications and/or verifications of the one or more abnormalities or anomalies in the one or more biological responses (e.g., the one or more actions, which may indicate fraudulent behavior) of the one or more subjects. In another variation, the system may identify that there are no abnormalities or anomalies related to one or more biological responses, allowing the system to verify the integrity of the competition. Multiple verifications may occur during the course of a competition to verify the integrity of the competition, with the number of verifications being a tunable parameter.

In a refinement, a predictive indicator or insight is created, modified, or enhanced to make one or more forecasts, predictions, probabilities, assessments, possibilities, projections, determinations or recommendations based upon the identification of the one or more targeted subjects, medical conditions, or biological responses. For example, in the context of a sporting event, the system may compare at least one unique asset derived from reference animal data and gathered animal data derived from the one or more source sensors, or its one or more derivatives (e.g., which may be another unique asset), and identify one or more abnormalities or anomalies related to one or more biological responses in the one or more patterns, trends, features, measurements, outliers, readings, characteristics, or the like (e.g., that a targeted individual is not exerting the amount of energy typically exerted by the targeted individual at that point in a match. Characteristically, the system can be configured to evaluate a plurality of animal data, non-animal data, or a combination thereof, simultaneously or concurrently via one or more artificial intelligence techniques in order to (1) create, modify, or enhance at least one unique asset, (2) enable one or more comparisons using the gathered data as contextual data for other animal data, or (3) a combination thereof. For example, in the context of a sport like tennis, the system may be configured to evaluate a plurality of data (e.g., types, sets) including, but not limited to, sensor-based animal data readings (e.g., positional data, location data, distance run, physiological data readings, biological fluid data readings, biomechanical movement data), non-animal data sensor data (e.g., humidity, elevation, and temperature for current conditions; humidity, elevation, and temperature for previous match conditions), length of points, player positioning on court, opponent, opponent's performance in specific environmental conditions, winning percentage against opponent, winning % against opponent in similar environmental conditions, current match statistics, historical match statistics based on performance trends in the match, head-to-head win/loss ratio, previous win/loss record, ranking, a player's performance in the tournament in previous years, a player's performance on court surface (e.g., grass, hard court, clay), length of a player's previous matches, current match status of a tennis player (e.g., athlete A is in Game 3 of Set 1 and is losing 5-2) and their historical data in the context of the current match status (e.g., all of athlete A match results when athlete A is in Game 3 of Set 1 and is losing 5-2, first serve percentage in second sets after playing n number of minutes, unforced errors percentage on the backhand side after hitting three n topspin backhands), and the like. Based upon the identification of the one or more biological responses, the system may make a prediction via the predictive indicator to predict whether the individual is purposefully engaging in abnormal behavior to affect the outcome of the match (e.g., it is 90% likely that the player is intentionally fixing the match). The system may also make a recommendation based upon the prediction (e.g., recommend that no more bets are to be taken on the match given the likelihood of match fixing) or insight.

In a refinement, examples of contextual data in the context of a sporting event that may be utilized by the system to 1) create, modify, or enhance at least one unique asset, (2) enable one or more comparisons using the gathered data as contextual data for other animal data, or (3) a combination thereof, can include, but are not limited to, traditional sports statistics collected during a competition/event (e.g., any given outcome data, including game score, set score, match score, individual quarter score, halftime score, final score, points, rebounds, assists, shots, goals, pass accuracy, touchdowns, minutes played, and other similar traditional statistics), in-competition data (e.g., whether the player is on-court vs off-court, whether the player is playing offense vs defense, whether the player has the ball vs not having the ball, the player's location on the court/field at any given time, specific on-court/field movements at any given time, who the player is guarding on defense, who is guarding the player on offense), streaks (e.g., consecutive points won vs lost; consecutive matches won vs lost; consecutive shots made vs missed), historical animal data (e.g., outcomes that happened which are cross-referenced with what was happening with the athlete's body—i.e., their biological responses—and factors surrounding it such as their heart rate and other heart-based information, body temperature data, distance covered/run data for a given point/game/match, positional data, biological fluid readings, hydration levels, muscle fatigue data, respiration rate data, any relevant baseline data, a player's biological data sets against any given team, who the player guarded in any given game, who guarded the player in any given game, the player's biological readings guarding any given player, the player's biological readings being guarded by any given player, minutes played, court/ground surface, the player's biological readings playing against any given offense or defense, minutes played, on-court locations and movements for any given game, other in-game data), comparative data to similar and dissimilar players in similar and dissimilar situations (e.g., other player stats when guarding or being guarded by a specific player, playing against a specific team) injury data (e.g., including history), recovery data (e.g., sleep data, rehabilitation data), training data (e.g., how the player performed in training in the days or weeks leading up to a game), nutrition data, a player's self-assessment data (e.g., how they're feeling physically, mentally, or emotionally), nutritional data, mental health data, and the like. Examples also include information such as round of competition (e.g., quarterfinal, finals), matchup (e.g., player A vs. player B; team A vs team B), date, time, location (e.g., specific court, arena, field, and the like), country of origin, crowd size, crowd noise levels, country of birth, age, weight, height, number of years associated with the event (e.g., number of years a player has been playing within a specific league), ranking or standing/seeding, height, weight, dominant hand or handedness (e.g., right hand dominant vs left hand dominant), equipment manufacturer, coach, habits, activities, genomic information, genetic information, medical history, family history, medication history, and the like. Examples of contextual data can also include the type of sport, career statistics (e.g., in the case of individual athletes in racquet sports as an example, number of: tournaments played, titles, matches played, matches won, matches lost, games played, games won, games lost, sets, sets won, sets lost, points played, points won, points lost, retirements, and the like). Examples of contextual information can also be scenario-specific. For example, in the sport of tennis, contextual information may be related to when a player is winning 2-0 or 2-1 in sets or losing 1-2 or 0-2 in sets, or time of day the player is playing, the type of event (e.g., big event vs exhibition), or the specific weather conditions the game is played in. Contextual information can also be related to head-to-head match ups. In the sport of squash for example, head-to-head information can be related to the number of head-to-head matches, games, number of times a player has been in a specific scenario vs the other player (e.g., in terms of game score: 3-0, 3-1, 3-2, 2-3, 1-3, 0-3, 2-0, 2-1, 1-2, 0-2, or retired). Examples of contextual information can also include how that player has performed in that particular tournament (e.g., matches played, matches won, games played, games won/lost, sets played, sets won/lost, court time per match, total court time, previous scores and opponents, and the like). Examples of contextual data can also include points won vs. points played, games (e.g., sets) won vs. games played, matches won vs. matches played, any given round rate (e.g., finals win/loss rate or semi-finals win/loss rate; number of times a player makes any given round in any given tournament (e.g., number of times a player makes the semifinals in any given tournament, which may on a yearly or career basis), title win rate (e.g., how many times the player has won this year or any given year or over a career; how many times a player has won that particular tournament), match retirement history, court surface (e.g., hard court vs clay court), and the like. Examples of contextual data can also include data such as environmental temperature data, court/field temperature data, humidity data, location, elevation data, and barometric pressure data, time, elevation data, location-based data, biomechanical-based data, physiological data, other biological data, and the like. It should be appreciated that such examples of contextual data in the context of a sports competition/event are merely exemplary and not exhaustive, and similar types of information can be collected for all sports and events. In the context of non-sporting events, similar types of contextual data and methodologies may be utilized. In another refinement, contextual data in the context of non-sports related events can also include outcome-related information that may or may not provide context to other data.

In some variations, the one or more computing devices (e.g., collecting computing device) may operate as a health monitoring system operable to provide animal data (e.g., signals, readings, computed assets, insights, predictive indicators, reference animal data, other metrics, and the like) for a single targeted individual or a plurality of targeted individuals (e.g., a family in a home, a group of patients in a hospital, an athlete on a sports team, an employee in a company, a participant in a workout class, and the like). The one or more computing devices may have a single point of communication with the one or more targeted individuals (e.g., a single display in which all the users interact with the one or more computing devices) or multiple points of communication (e.g., multiple displays operated by the system, which may include one or more phones, smart watches, mountable head units, smart speakers, tablets, monitors, or other displays which may be operable to interact with the one or more computing devices and operate as an extended display for the one or more computing devices). With the one or more targeted individuals being monitored via one or more sensors simultaneously, the system may be configured to provide all animal data metrics for all targeted individuals, selectable (or subset) animal data metrics for all targeted individuals, all animal data metrics for select targeted individuals, or selectable (or subset) animal data metrics for select targeted individuals. In a refinement, the one or more computing devices may be configured to automatically provide one or more animal data metrics via a display or other communication mechanism (e.g., audio, send the data to another device such as a smart watch, mobile phone, smart speakers, head mountable unit such as a augmented reality display or smart glasses, and the like) based upon one or more actions taken by the one or more targeted individuals (e.g., a targeted individual approaching the display device which triggers the display device to provide their animal data metrics; scanning an object or machine-readable image associated with a targeted individual which in turn triggers the computing device to display their animal data metrics; selecting a targeted individual profile for metrics display on the computing device; verbally communicating to the computing device to provide the one or more metrics; and the like). In a variation, the one or more metrics may be provided via the one or more displays via an alert (e.g., the system sends an alert to an individual's mobile display such as their phone, smart watch, earbuds, head mountable unit, or the like). In a refinement, the system can identify an individual based upon their unique asset, the one or more sensors, their animal data, or a combination thereof and automatically provide the one or more animal data metrics to a display device. In another refinement, the computing device can identify an individual amongst a group of individuals based upon their unique asset, the one or more sensors, their animal data, or a combination thereof, and automatically provide the one or more animal data metrics to a display device. For example, a targeted individual in a group of targeted individuals being monitored simultaneously (e.g., a family being monitored in a home via the same monitoring system) may physically approach the display device. Upon approach, the system may be configured to automatically identify the targeted individual based upon their unique asset, their one or more sensors, their animal data (e.g., facial recognition, voice recognition, fingerprint scan, other animal data), or a combination thereof. Based upon the identification and verification of the targeted individual, the system may be configured to automatically display the individual's animal data and derivatives. This can be advantageous, for example, in situations where an administrator of the system does not want to provide access to animal data for all monitored individuals to all users (e.g., which may be the monitored individuals) accessing the system. In a refinement, the system or individual can determine when to display the data, what content (e.g., animal data) to display, the frequency of the data display, the format of the display, or a combination thereof, for a targeted individual based on one or more defined variables (e.g., proximity of one or more other individuals, location of the individual in relation to the display, and the like). This can be a tunable parameter. For example, the system may be operable to locate the positioning or distance of one or more other sensors that are not a source sensor (e.g., on the person of an non-targeted individual, such as a sensor being worn) in relation to the display device or in relation to the one or more source sensors being utilized (e.g., worn) by the targeted individual and determine the timing for when the animal data should be displayed (e.g., the system senses a sensor on the targeted individual and also senses another sensor on another non-targeted individual in close proximity and within n feet of the targeted individual, so the system decides not to display the data, or change the content of the display until the non-targeted individual is located at a pre-determined distance away from the targeted individual). In another example, the system may be operable to sense when the targeted individual is alone compared to when the targeted individual is with one or more other individuals (e.g., using facial recognition sensors, infrared sensors, proximity sensors, and the like).

In some variations, the system may be utilized to identify and/or verify (e.g., authenticate) the identity of one or more subjects featured via video, virtual/holographic environment, or other environment where the targeted subject is not physically present. This may occur by the system collecting animal data from at least one sensor that is synced with a data capturing system such as a video or visual capturing device or audio capturing device. The system can generate one or more unique assets to validate the identity of the subject based upon their animal data and validate that the video or audio is, in fact, featuring the subject (e.g., and not a deepfake or digitally manipulated video or audio of the subject). In some variations, the system may display or communicate one or more indicators (e.g., on-screen verifier) that represents a verification or authentication that a targeted subject featured in a video has been, in fact, verified by the system to be the actual subject (e.g., in the case of verifying the identity of a human subject being represented by an avatar in a virtual environment, the system can verify that the avatar in the virtual environment is, in fact, being operated by the targeted human subject and not another individual by verifying the identity of the subject representing the avatar via their animal data and/or its one or more derivatives, and providing one or more indicators on-screen—such as a check mark associated with the avatar's profile—or via other communication mechanism to inform other users of this verification). In a refinement, the system can use animal data derived from one or more sensors (e.g., one or more computed assets) to validate the identity of the subject and validate that the video (e.g., including virtual/holographic environment) or audio is, in fact, featuring the subject (e.g., using a metric like heart rate to confirm that the subject does, in fact, have a biological reading and is therefore not a deepfake video of the subject). The system may operate in conjunction with one or more media systems to provide continuous or intermittent verifications of the one or more biological-based signals or readings derived from the subject in the video or audio. In a variation, the system may include a mechanism to identify and/or verify a real biological signal or reading from a subject compared to a digitally-created or altered signal (e.g., a synthetic signal) that may be generated as part of the deepfake process.

The animal data-based identification and recognition system can also be utilized in a variety of other ways, including: (i) as part of healthcare system to identify whether an individual has any given medical condition based on the collection of their animal data; (ii) as part of a verification system for content or media platforms to identify, verify, and/or authenticate the one or more targeted individuals authorized to access one or more streaming services (e.g., as a security layer to prevent password sharing with a streaming service); (iii) as part of an insurance or monetization system to identify the targeted individual, and verify that the one or more sensors are, in fact, collecting data from the targeted individual; (iv) as part of a security-based system to identify, verify, and/or authenticate the one or more targeted individuals, sensors, medical conditions, or biological responses; and the like.

In some variations, the system may not have verified that the animal data is from a targeted individual but may have verified that the animal data is associated with a specific medical condition or biological response. In this example, the system may also associate one or more attributes to the animal data that do not identify the targeted individual but provide context to the animal data such as age, weight, height, previous medical history, and the like. This may also apply to reference animal data whereby the system may associate one or more attributes with animal data that do not specifically identify a person but can identify one or more medical conditions or biological responses. In another variation, in the event the system is unable to verify the one or more individuals, medical conditions, or biological responses in the reference animal data, the system may associate multiple individuals, multiple medical conditions, or multiple biological responses to the same reference animal data, the same one or more digital records, or a combination thereof. For example, the system may not be able to identify whether a specific individual's record is derived from a specific individual but may have n number of individuals that may be the source of the reference animal data (e.g., narrow down from a big subset to a smaller subset). Similarly, the system may not be able to identify a specific medical condition based on the reference animal data but may provide multiple medical conditions that may be associated with the reference animal data. In a refinement, the system can provide negative identification to one or more individuals (e.g., based upon the collected animal data from the one or more source sensors, the system can eliminate one or more reference individuals from being identified as the targeted subject).

In some variations, the system collects reference animal data from one or more computing devices (e.g., third party computing devices) that is associated with one or more reference individuals. In a refinement, the system is operable to verify the association between the one or more reference individuals and the collected reference animal data from one or more computing devices. For example, the system may receive reference animal data from another system that is identified from being derived from a reference individual. To verify this, the system may utilize reference animal data form the reference individual currently in the system and create two or more unique assets—at least one unique asset from the current reference animal data and at least one unique asset from the collected reference animal data from the one or more computing devices—to verify that the reference animal data from one or more computing devices is in fact associated with the correct reference individual.

As described herein, the animal data-based identification and recognition system may be implemented as part of a monetization system (e.g., animal data-based monetization system), insurance or health system (e.g., system for an insurance or health-based company to verify that the animal data being collected is from the targeted individual in order to create, modify, or enhance one or more products or services), sports wagering/integrity system, or any other type of system whereby animal data can be used (and useful) to identify and/or verify an individual, a medical condition, or biological response. In a refinement, upon one or more identifications or verifications, the system is configured to take one or more actions, the one or more actions including at least one of: (1) evaluating, assessing, preventing, or mitigating animal data-based risk; (2) creating, modifying, enhancing, acquiring, offering, or distributing one or more products (e.g., insurance products, sports wagering products, health-based products); (3) evaluating, assessing, or optimizing animal data-based performance for a targeted individual; (4) formulating one or more strategies; (5) mitigating or preventing one or more risks; (6) evaluating, creating, calculating, deriving, modifying, enhancing, or communicating one or more recommendations (e.g., recommending one or more actions based upon the medical condition), predictions, probabilities, odds, or possibilities; (7) creating, modifying, enhancing, or accepting one or more wagers; or (8) a combination thereof. For example, an insurance company may operate an application on computing device 20 to collect sensor-based animal data from targeted individual 16 for the purposes of creating, modifying, or enhancing one or more insurance products, adjusting one or more insurance premiums, providing one or more quotations for one or more products or services, acquiring animal data (e.g., as training data) for consideration for one or more use cases, and the like. The individual may identify themselves to the system as an assumed subject through one or more selection or input options. The system is configured to establish communication with the one or more sensors being utilized by the individual (e.g., worn, accessed) and collects animal data. The system is also configured to collect metadata (e.g., non-sensor based animal data, non-animal data) to provide context to the sensor-based animal data. The system creates at least one unique asset based upon the collected animal data and contextual data. The system also creates, modifies, enhances one or more unique assets from the reference animal data, or gathers one or more previously created unique assets from the reference animal database, based upon (1) the type of animal data being collected by the one or more source sensors; (2) the one or more types of sensors; (3) the one or more operating parameters or characteristics associated with each source sensor; (4) the metadata (e.g., one or more external factors including activity in which data is collected, time, environmental conditions, location, and the like; one or more attributes related to the individual); (5) the types of animal data in the reference animal database and its associated metadata; (6) the sources of animal data in the reference animal database; or (7) a combination thereof. For example, the system may select reference animal data to create the one or more unique assets that matches the sensor-based animal data and metadata in terms of data type (e.g., utilizing only animal data derived from the one or more source sensors), sensor type (e.g., utilizing only data derived from the same or similar sensors), metadata characteristics (utilizing reference animal data that matches the context in which the sensor-based data was captured), and the like. Characteristically, the system is configured to compare the two or more unique assets. The comparison identifies the assumed individual as the targeted individual, enabling the insurance company to associate the animal data with the individual and verify that the sensor-based animal data being collected by the system is derived from the targeted individual. In a refinement, the system may conduct multiple verifications during the data collection period to ensure that the animal data is being collected from the targeted individual. This in turn enables the insurance company to provide an insurance quote to the targeted individual based upon their animal data (e.g., how much an insurance plan would cost based upon their current animal data readings and health condition), adjust one or more premiums for the targeted individual based upon their animal data (e.g., if the animal data from the targeted individual is providing favorable readings, the insurance company may lower the premium; if the if the animal data is providing readings that show one or more health issues with the individual, the insurance company may raise the premium), create one or more insurance products for the targeted individual based upon their animal data (e.g., customized insurance products based upon the targeted individual's animal data readings), and the like.

In the previously described variations, the at least one source sensor is oftentimes associated with an individual prior to identification/verification and gathering animal data. Additionally, reference animal data is oftentimes available, thus enabling one or more unique assets to be created, modified, or enhanced. However, in some cases, the at least one source sensor may not be initially associated with an individual and not gathering animal data from the outset (e.g., the at least one source sensor may be powered on to communicate with one or more computing devices or other sensors but is not gathering data from the individual). In other cases, the at least one source sensor may be initially associated with a targeted individual but not gathering animal data from the outset. In other cases, the at least one source sensor may be initially associated with a targeted individual and gathering animal data from the outset (e.g., providing data to a collecting computing device) but no reference animal data from the targeted individual is available. Such variations can make it difficult to ensure that (1) the one or more source sensors are associated with the targeted individual (e.g., on the body of the correct/desired targeted individual, set up to collect data from the correct/desired targeted individual), (2) the one or more sensors are (in fact) collecting data from the targeted individual, and (3) the identity of the individual from whom the animal data is being derived is confirmed as the targeted individual.

While the previously described subject matter details multiple variations of a system which uses animal data collected from one or more sensors to create one or more unique assets to determine the identification of individual, one or more medical conditions or one or more biological responses, the system can also be configured to provide one or more solutions for the one or more other variations. Specifically, the system may be implemented to verify under a variety of conditions: (1) that the one or more source sensors are correctly associated with the desired targeted individual (e.g., on the body of the correct targeted individual, set up to collect data from the desired targeted individual), (2) the one or more source sensors are (in fact) collecting data from the desired targeted individual, or (3) the identity of the targeted individual (e.g., confirming the stated identity of the targeted individual). Such variations of the system may utilize two or more sensors, one or more of which are primary sensors used as the one or more source sensors to gather animal data from a targeted individual, and one or more of which are secondary sensors used, at least in part, to make one or more verifications related to the at least one primary sensor and/or the targeted individual. In a refinement, the one or more secondary sensors are also source sensors. These variations of the system may be implemented as part of an animal data-based animal data-based identification and recognition system, or as part of a sensor authentication and verification system, enabling one or more verifications that the at least one primary sensor is gathering data from, or associated with, the correct targeted individual, as well as verification of the targeted individual themselves (e.g., verification of their identity).

In one embodiment, a sensor authentication and verification system is comprised of at least one primary source sensor operable to gather animal data from one or more targeted individuals and provide the animal data to a collecting computing device, the at least one primary source sensor being further operable to receive one or more signals (e.g., receive command signals or instructions to take or perform an action) wherein the one or more signals and animal data are transmitted electronically. The collecting computing device is operable to: (1) gather information (e.g., animal data, including characteristics/attributes; non-animal data) related to the targeted individual (e.g., inputted or selected by the targeted individual or other user; gathered from the targeted individual via one or more source sensors or from another computing device), with the information including one or more identifiable characteristics (e.g., attributes) of the targeted individual; (2) send one or more signals to, and receive information from, the at least one primary source sensor; and (3) gather information from the one or more targeted individuals and the at least one primary source sensor via one or more secondary sensors. Both the primary and secondary sensors can be biosensors that collect animal data. In some variations, the one or more primary and secondary sensors can also collect non-animal data. In a refinement, the at least one primary source sensor is operable to send one or more signals (e.g., send a command to another sensor or computing device).

The collecting computing device establishes communication with the at least one primary sensor and sends one or more signals to the at least one primary sensor to take one or more actions. The collecting computing device also establishes communication with the one or more secondary sensors and sends one or more signals to collect information from, or related to, the at least one primary sensor, the targeted individual, or both. In a refinement, the collecting computing device sends one or more signals to the one or more secondary sensors, the one or more signals initiating the one or more secondary sensors to send one or more signals to the at least one primary sensor. The one or more actions taken by the at least one primary sensor are captured (e.g., identified, observed) by the one or more computing devices, the one or more secondary sensors, or both, verifying the at least one primary source sensor's association with the one or more targeted individuals. In a variation, the collecting computing device is configured to take one or more coordinated actions (e.g., processing steps) based upon the one or more actions taken by the at least one primary source sensor and at least a portion of the gathered information from the one or more secondary sensors to verify the at least one primary source sensor's association with the one or more targeted individuals.

In another variation, the collecting computing device gathers animal data from the one or more secondary sensors from which at least one characteristic of the one or more identifiable characteristics related to the targeted individual is identified using reference animal data (e.g., which may be reference data from the targeted individual, reference data from another one or more subjects, or a combination thereof) to verify the at least one characteristic. In a refinement, one or more signals (e.g., commands) may be provided (e.g., sent) to the at least one primary sensor via the collecting computing device either directly or indirectly (e.g., via the one or more secondary sensors) to initiate animal data collection from the at least one primary sensor, from which at least one characteristic of the one or more identifiable characteristics related to the targeted individual is identified. In another refinement, upon verification of the at least one primary source sensor's association with the one or more targeted individuals and the at least one characteristic, at least a portion of the animal data gathered from the at least one primary source sensor is distributed to one or more computing devices for consideration. In another refinement, at least a portion of the data derived from the one or more secondary source sensors is distributed with animal data derived from the at least one primary source sensor to one or more computing devices for consideration. In another refinement, the collecting computing device is a computing device with at least one integrated, attached, connected, or affixed sensor comprised of one or more sensors. In another refinement, the collecting computing device is comprised of two or more computing devices.

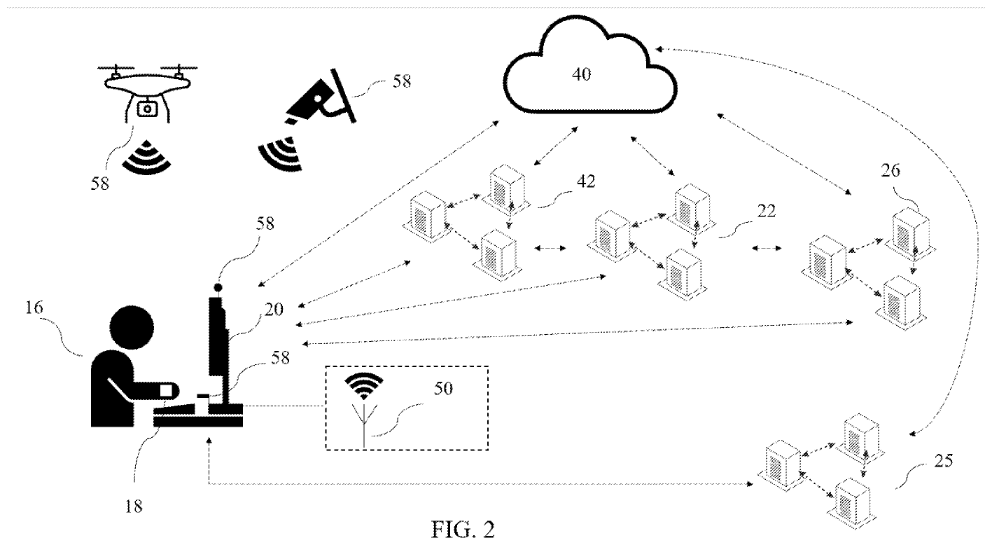

FIG. 2 provides a schematic of a sensor authentication and verification system related to animal data. Moreover, FIG. 2 features multiple embodiments of the invention. In one embodiment, the at least one primary sensor 18 (e.g., which can be wearable, although not required), while operable to collect animal data, is not collecting data—at least initially—from targeted individual 16. Furthermore, the at least one primary sensor 18 may or may not be associated with targeted individual 16 in the system. To verify the association between the at least one primary sensor 18 and targeted individual 16 (e.g., to confirm that the at least one primary sensor 18 is, in fact, the data gathering source sensor for the targeted individual 16; to confirm the at least one primary sensor 18 is ready to collect animal data from the targeted individual 16), targeted individual 16 accesses the system (e.g., logs into the system via one or more data collection programs) via one or more computing devices 20 (which can include hardware transmission subsystem 50) and provides the system with at least one identifiable characteristic (e.g., attribute) of the targeted individual (e.g., age, weight, height, facial characteristics, bodily defects, gender, a medical condition, and the like) that can be verified (e.g., via measurement, observation, gathering) by one or more sensors. In one variation, this can be achieved by a user (e.g., which may be the targeted individual) inputting one or more attributes into the system. In another variation, the system may automatically collect the at least one identifiable characteristic of targeted individual 16 from the one or more secondary sensors 58 (e.g., in one example, secondary sensor 58 may be an optical sensor that captures facial recognition data and identifies or predicts the age, gender, or other characteristics of the targeted individual 16 using one or more AI techniques; secondary sensor 58 may be a biometric fingerprint scanner that identifies targeted individual 16) or via one or more other computing devices once at least one identifiable characteristic (e.g., name) is provided, which may occur via access to another program (e.g., digital identification or health card via an application that provides identifiable characteristics). In a refinement, one or more other computing devices that gather and/or provide one or more identifiable characteristics may include one or more scanning or communication devices that gather and/or provide such information. The system takes one or more actions to establish communication with the at least one primary sensor 18 to have the at least one primary sensor 18 take one or more actions to identify itself to the system (e.g., the system makes sensor 18 ping the system, flash a light, make a sound, vibrate, provide a signal, or take another one or more actions that enable identification of the sensor by the system). In a refinement, the one or more actions may be communicated to the at least one primary sensor 18 by computing device 20 via one or more secondary sensors 58. Characteristically, the at least one primary sensor's one or more actions and characteristically are identifiable by the one or more secondary sensors 58. The system then uses one or more secondary sensors 58 (e.g., which can be a computing device featuring one or more secondary sensors, standalone wireless or wired sensors or wired sensors in communication with the one or more computing devices, and the like) to confirm the at least one primary sensor 18's association with (e.g., which may include its availability to collect animal data from) targeted individual 16 (e.g., the one or more secondary sensors 58 may be an optical sensor that captures visual information to confirm that the at least one primary sensor is on the body of targeted individual 16, the one or more secondary sensors further capturing an action taken by primary sensor 18—e.g., the secondary optical sensor captures the flashing of a light derived from the primary sensor, the secondary optical sensor receives a signal or other form of communication from the primary sensor that enables identification of the primary sensor). In a refinement, the one or more secondary sensors 58 send one or more signals to the at least one primary sensor 18 that initiates one or more actions by the at least one primary sensor 18 that are captured by one or more secondary sensors 58 to verify the at least one primary sensor's association with targeted individual 16. Upon verification of the at least one primary sensor's association with the targeted individual, the system can assign the at least one primary sensor to the targeted individual.

In a refinement, upon verification of the association between the at least one primary sensor and the targeted individual, the system verifies the one or more identifiable characteristics provided by or gathered from the targeted individual. In one variation, the system can gather animal data from the targeted individual and verify the one or more identifiable characteristics by creating one or more unique assets based upon animal data gathered from the one or more source sensors (e.g., primary sensors 18, secondary sensors 58) and evaluating (e.g., comparing) the one or more unique assets with one or more unique assets from one or more subjects derived from reference animal data. The system can identify the targeted individual based upon their animal data, enabling a verification of the one or more identifiable characteristics based upon the information available in the reference animal data database (i.e., via one or more computing devices 25). In another variation, reference animal data for the targeted individual may not be available or exist. In this scenario, the system utilizes reference animal data derived from one or more computing devices 25 from other individuals to evaluate (e.g., compare, cross-reference) the collected animal data from the at least one primary sensor 18, the one or more secondary sensors 58, or a combination thereof with animal data from individuals sharing the at least one characteristic of the provided or gathered one or more identifiable characteristics to verify the one or more identifiable characteristics of the targeted individual (e.g., the system may initiate data collection from the at least one primary sensor or use the one or more secondary sensors to gather at least one biometric authentication data—for example, facial recognition data, DNA sequencing/matching data, fingerprint data, hand geometry data, voice recognition data, keystroke dynamics data including usage patterns on computing devices such as mobile phones, signature recognition data, ear acoustic authentication data, eye vein recognition data, iris recognition data, retinal scan data, finger vein recognition data, footprint and foot dynamics data, body odor recognition data, palm print recognition data, palm vein recognition data, skin reflection data, thermography recognition data, speaker recognition data, gait recognition data, lip motion data—and/or one or more types of physiological, biomechanical (e.g., gait/posture), and/or other observational animal data (e.g., bodily defects, tattoos, skin disorders, hair, and the like) to confirm the one or more characteristics—such as age or a medical condition—provided by the targeted individual without having any historical data on the targeted individual but having historical data on one or more individuals that are similar—via the at least one shared characteristic—to the targeted individual to verify their one or more characteristics). For example, based upon observable information gathered by the one or more secondary sensors 58 (e.g., one or more optical sensors), the system may determine—based upon the reference animal data accessible by the system—that targeted individual 16 is a male between 65-75 years old with a specific type of skin disease. The system can then compare these one or more gathered characteristics with the information provided by targeted individual 16 (e.g., age, sex, and medical condition) to verify the provided information (e.g., the one or more characteristics). One or more of the steps or actions taken by the one or more computing devices or the one or more sensors may occur utilizing one or more artificial intelligence techniques. In a variation, the system verifies the one or more characteristics provided by the targeted individual (or gathered by the system via the one or more secondary sensors or other computing devices) by utilizing one or more artificial intelligence techniques to verify the one or more characteristics with reference animal data. The verification of the one or more characteristics enables verification, at least in part, of the individual.

In a refinement, at least one of the one or more secondary sensors 58 is a biosensor that gathers, or provides information that can be converted into, at least one of the following types of animal data: facial recognition data, eye tracking & recognition data, blood flow data, blood volume data, blood pressure data, biological fluid data, body composition data, biochemical data, pulse data, oxygenation data, core body temperature data, skin temperature data, galvanic skin response data, perspiration data, location data, positional data, audio data, biomechanical data, hydration data, heart-based data, neurological data, genetic data, genomic data, skeletal data, muscle data, respiratory data, kinesthetic data, ear acoustic authentication data, finger vein recognition data, fingerprint recognition data, footprint and foot dynamics data, hand geometry data, body odor recognition data, palm print recognition data, palm vein recognition data, skin reflection data, thermography recognition data, keystroke dynamics data (e.g., including usage patterns on computing devices such as mobile phones), signature recognition data, speaker recognition data, voice recognition data, gait recognition data, lip motion data, medical condition data, biological response data, or characteristic/attribute data. In another refinement, at least one of the one or more secondary sensors 58 is operable to collect information related to the one or more primary sensors 18 to verify their association with the targeted subject.

In a variation, the at least one primary sensor 18 is associated with targeted individual 16 but the at least one primary sensor 18 is not collecting animal data from the targeted individual. In this scenario, the one or more identifiable characteristics that the system has collected related to the targeted individual from one or more other individuals (e.g., including groups of individuals) can enable a more refined search with the reference animal data to more accurately verify the one or more characteristics provided by, or related to, the targeted individual. In this regard, verification can be characterized by at least one of: a percentage match, possibility, probability, prediction, confidence indicator (e.g., degree of confidence), score (e.g., accuracy score, precision score, and the like), or likelihood (e.g., 78% likelihood that the targeted individual is a specific known subject). Characteristically, a verification can include a positive verification (e.g., 100% match) or partial positive verification, meaning the verification is not absolute (e.g., n % match that is less than 100%). In the event there is limited reference animal data available, the system may still be operable to make one or more identifications and/or verifications based upon data collection from the one or more secondary sensors.

In another variation, the at least one primary sensor is associated with the targeted individual and collecting animal data from the targeted individual but there is no reference animal data specific to the targeted subject. In this scenario, at least one unique asset can be created, modified, or enhanced using at least a portion of the targeted individual's animal data and one or more characteristics related to the targeted subject. The at least one unique asset can be compared with at least one unique asset created from the reference animal data that is created, modified, or enhanced using at least one or more of the same parameters based upon animal data (e.g., ECG and voice data for an individual of a specific age, weight, and medical history) derived from one or more other individuals that share at least one characteristic with the targeted individual to verify, at least in part, the one or more characteristics provided by or related to the targeted individual, as well as the association between the targeted individual and the at least one primary sensor.

In another variation, the system makes a plurality of verifications. For example, the system may make an additional one or more verifications after the association between the individual and primary sensor are identified/verified to ensure the at least one primary sensor is collecting animal data from the desired targeted individual in a future time period. This feature is particularly advantageous when animal data is streaming from the at least one primary sensor (e.g., continuously or intermittently), enabling continuous verification of the association between the targeted individual and the at least one primary sensor. The frequency of the one or more verifications is a tunable parameter. In a refinement, the verification of the association between the targeted individual and the at least one primary sensor occurs utilizing one or more secondary sensors. In another refinement, when the at least one primary sensor generates one or more readings or signals determined by the system to be abnormal or uncharacteristic for the targeted individual based upon the collected data and reference animal data, the system initiates the one or more secondary sensors to (1) confirm the association between the one or more sensors and the targeted individual, (2) gather contextual information related to the at least one primary sensor and the targeted individual to determine one or more causes of the one or more abnormal readings or signals, or both. In some cases, upon the one or more secondary sensors gathering information, one or more steps related to identification and/or verification of the at least one primary sensor and/or the targeted individual may be initiated.

In another variation, in the event the one or more computing devices 20 have not associated targeted individual 16 with the at least one primary sensor 18 or are seeking verification of association, the one or more computing devices 20 may provide one or more instructions via the display for targeted subject 16 to take an action, such as exhibit a biological response (e.g., activity such as standing up and down, jumping, moving side to side, accelerate breathing rate). Computing device 20 may then send one or more commands to the at least one primary sensor 18 to initiate animal data collection. In a refinement, the one or more commands can include to start collecting data. The system may utilize reference animal data (e.g., derived from one or more computing devices 25) to evaluate the animal data collected from the primary source sensor (e.g., xyz data that provides mobility/biomechanical data, evaluate breathing patterns) with the reference animal data based upon the one or more instructions provided via the display in order to identify that the at least one primary sensor is gathering data from an individual exhibiting those characteristics and enable an initial association between the targeted subject and at least one primary sensor. At this point, the sensor could have been utilized by another individual exhibiting the same biological response as the targeted subject, thereby enabling the system to make an incorrect association between subject and sensor. Therefore, the system initiates the at least one secondary sensor 58 to gather information from which one or more calculations, computations, measurements, derivations, extractions, extrapolations, simulations, creations, combinations, modifications, enhancements, estimations, evaluations, inferences, establishments, determinations, conversions, deductions, or observations are made to verify the targeted individual's action (e.g., did the targeted individual exhibit the biological response; was there another individual in proximity that exhibited the same biological response; did that individual utilize the same primary sensor), verify the one or more provided or gathered identifiable characteristics of the targeted individual (e.g., the secondary sensor verifies that the targeted individual is a 40-45 year old male with a birth defect on the left arm), verify that the at least one primary sensor is gathering data from the targeted individual, or a combination thereof. In a refinement, the one or more computing devices or secondary sensors may send one or more commands to the at least one primary sensor to take one or more actions while collecting animal data to enable the at least one primary sensor to identify itself as the at least one primary sensor collecting animal data from the targeted individual.

In another embodiment, a sensor authentication and verification system includes one or more primary source sensors 18 operable to gather animal data wherein the animal data is transmitted electronically. A collecting computing device 20 is operable to send one or more commands to the one or more primary source sensors 18. The collecting computing device 20 is further operable to gather information from or related to a targeted individual 16, with the information including (1) at least one identifiable characteristic/attribute related to the targeted individual (e.g., inputted or selected by the targeted individual or other user; gathered from the targeted individual via one or more source sensors or another computing device), and (2) animal data from two or more sensors, at least one of which is a primary source sensor 18 and at least one of which is a secondary source sensor 58. In some variations, the system may not be operable initially to gather animal data from the at least one primary source sensor, or may not have initially identified the primary source sensor as being associated with the targeted individual (e.g., amongst a group of sensors), or may not have verified that the at least one primary source sensor is ready to collect animal data from the targeted individual (e.g., it may be unclear whether the sensor is ready to collect data from the targeted individual versus another individual). The collecting computing device sends one or more commands to at least one primary source sensor to perform one or more actions (e.g., collect animal data, perform a function that enables identification of the sensor), the collecting computing device further receiving animal data, non-animal data (e.g., information about the at least one primary sensor), or a combination thereof from one or more secondary source sensors. Characteristically, the at least one primary source sensor's performance of the one or more actions and the collecting computing device's gathering of animal data from the one or more secondary source sensors occurs simultaneously or in succession (e.g., the one or more actions from the primary source sensor are captured and recorded by the secondary source sensor which is then provided back the collecting computing device; animal data is collected by both the primary and secondary sensors simultaneously and evaluated by the system to determine the primary source sensor's association with the targeted individual). In one variation, the collecting computing device sends one or more commands to at least one primary source sensor to perform one or more actions, the one or more actions occurring while the one or more secondary source sensors gather information related to the one or more actions simultaneously (e.g., which may include animal data, non-animal data, or a combination thereof), the one or more secondary source sensors further providing the gathered information to the collecting computing device. The collecting computing device receives information from the at least one primary source sensor and the one or more secondary source sensors related to the one or more actions taken by the at least one primary source sensor. The collecting computing device takes one or more processing steps (e.g., syncing data via time stamps, other processing steps) and evaluates (e.g., analyzes) the one or more actions with the received information (e.g., animal data, non-animal) from the one or more secondary source sensors to verify the origin of the animal data, the origin including at least one primary source sensor, the targeted individual, or both. Additionally, the collecting computing device takes one or more processing steps utilizing reference animal data (e.g., via one or more computing devices 25) and at least a portion of the animal data collected from the at least one primary source sensor, the one or more secondary source sensors, or a combination thereof to verify the at least one characteristic/attribute related to the targeted individual (e.g., the targeted individual provides to the system that they are 70 years old; the system verifies that the targeted individual is, in fact, 70 years old based upon an evaluation of the animal data collected from the one or more sensors—for example, an evaluation of their vitals derived from the one or more sensors and evaluation of their facial recognition data, hair color, and posture). The verification of the at least one characteristic/attribute of the targeted individual and the origin of the animal data enables the system to verify the association between the at least one primary source sensor and the targeted individual. In a refinement, the collecting computing device is a computing device with at least one integrated, attached, or affixed at least one sensor comprised of one or more sensors. In another refinement, the collecting computing device is comprised of two or more computing devices. In another refinement, at least a portion of the verified animal data is distributed by collecting computing device 20 to another one or more computing devices (e.g., one or more computing devices 26 or 42) for consideration, which may occur directly, via cloud 40, via intermediary server 22, or a combination thereof. In another refinement, data derived from the one or more secondary source sensors 58 is distributed with animal data derived from the at least one primary source sensor 18 to one or more computing devices for consideration.

In another embodiment, a sensor authentication and verification system includes one or more source sensors operable to gather continuous or intermittent animal data wherein the animal data is transmitted electronically. A collecting device is operable to send and receive one or more commands to and from one or more primary source sensors to confirm the identity of the one or more primary source sensors. The collecting computing device is further operable to gather animal data from two or more sensors, with at least one sensor gathering physiological data from the targeted individual and at least one sensor gathering biometric authentication data. The collecting computing device takes one or more coordinated processing steps with at least a portion of the animal data derived from two or more sensors to verify the origin of the animal data, the origin including at least one primary source sensor. In a refinement, at least a portion of the verified animal data is distributed to another one or more computing devices for consideration. In another refinement, at least a portion of the verifying animal data Is distributed with the verified animal data to another one or more computing devices for consideration. In another refinement, biometric authentication data includes at least one of: facial recognition data, DNA matching data, fingerprint data, hand geometry data, voice recognition data, keystroke dynamics data—including usage patterns on computing devices such as mobile phones, signature recognition data, ear acoustic authentication data, eye vein recognition data, iris recognition data, retinal scan data, finger vein recognition data, footprint and foot dynamics data, body odor recognition data, palm print recognition data, palm vein recognition data, skin reflection data, thermography recognition data, speaker recognition data, gait recognition data, lip motion data, medical condition data, biological response data, or characteristic/attribute data.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. An animal data-based identification and recognition system comprising:
one or more source sensors that gather animal data from an assumed or unknown subject wherein the animal data is transmitted electronically;
one or more computing devices configured to collect the animal data from the one or more source sensors, wherein;
the one or more computing devices are also configured to gather reference animal data related to a targeted subject, a targeted medical condition, or a targeted biological response;
the one or more computing devices are also configured to create, modify, or enhance at least one unique asset related to the targeted subject, the targeted medical condition, or the targeted biological response based upon the reference animal data;
the one or more computing devices are configured to perform a comparison by comparing the at least one created, modified, or enhanced unique asset with at least a portion of the animal data derived from the one or more source sensors, or its one or more derivatives, from the assumed or unknown subject; and
the comparison between the at least one created, modified, or an enhanced unique asset and the animal data derived from the one or more source sensors, or one or more derivatives thereof, identifies the targeted subject, the targeted medical condition, or the targeted biological response.

2. The animal data-based identification and recognition system of claim 1 wherein the comparison to identify the targeted subject, the targeted medical condition, or the targeted biological response occurs between two or more unique assets, at least one of which is a created, modified, or the enhanced unique asset from the animal data derived from the one or more source sensors.

3. The animal data-based identification and recognition system of claim 2 wherein the two or more unique assets identify the targeted subject, one or more medical conditions, or one or more biological responses.

4. The animal data-based identification and recognition system of claim 2 wherein the two or more unique assets identify the targeted subject and one or more medical conditions, biological responses, or a combination thereof.

5. The animal data-based identification and recognition system of claim 1 wherein an identification is characterized by at least one of a percentage match, possibility, probability, prediction, confidence indicator, score, or likelihood.

6. The animal data-based identification and recognition system in claim 1 wherein the assumed subject is a known or presumed subject.

7. The animal data-based identification and recognition system of claim 1 wherein upon identification of the targeted subject, the targeted medical condition, or the targeted biological response by the one or more computing devices, the one or more computing devices make one or more verifications.

8. The animal data-based identification and recognition system of claim 7 wherein the one or more computing devices verify an identity of the targeted individual, the targeted medical condition, or the targeted biological response.

9. The animal data-based identification and recognition system of claim 7 wherein the one or more computing devices verify an association between the targeted individual and the one or more source sensors.

10. The animal data-based identification and recognition system of claim 7 wherein the one or more computing devices verify that the one or more source sensors are collecting data from the targeted individual.

11. The animal data-based identification and recognition system of claim 7 wherein the one or more computing devices verify the one or more tags associated with the targeted individual, the one or more source sensors, the animal data, one or more medical conditions, one or more biological responses, or a combination thereof.

12. The animal data-based identification and recognition system of claim 7 wherein the one or more computing devices verify an association between the targeted individual and the animal data from the one or more source sensors.

13. The animal data-based identification and recognition system of claim 12 wherein upon verification, at least a portion of the animal data from the verified subject is distributed by the one or more computing devices to one or more other computing devices for consideration.

14. The animal data-based identification and recognition system of claim 13 wherein the animal data is distributed as part of an animal data monetization system.

15. The animal data-based identification and recognition system of claim 7 wherein a plurality of verifications occur based upon new animal data entering the animal data-based identification and recognition system via the one or more computing devices.

16. The animal data-based identification and recognition system of claim 7 wherein the one or more computing devices generate one or more alerts based upon one or more identifications or verifications.

17. The animal data-based identification and recognition system of claim 1 wherein upon identification of the targeted subject, the targeted medical condition, or the targeted biological response by the animal data-based identification and recognition system, the animal data-based identification and recognition system associates at least a portion of the animal data derived from the one or more source sensors, or the one or more derivatives thereof, with the targeted subject, the targeted medical condition, or the targeted biological response.

18. The animal data-based identification and recognition system of claim 1 wherein upon identification of the targeted subject, the targeted medical condition, or the targeted biological response by the one or more computing devices, the one or more computing devices create, modify, assign, or a combination thereof, one or more tags.

19. The animal data-based identification and recognition system of claim 1 wherein the comparison between the at least one created, modified, or the enhanced unique asset and the animal data derived from the one or more source sensors, or one or more derivatives thereof, verifies an origin of the animal data derived from the one or more source sensors, or one or more derivatives thereof.

20. The animal data-based identification and recognition system of claim 19 wherein the origin is the targeted subject.

21. The animal data-based identification and recognition system of claim 19 wherein the origin is the one or more source sensors.

22. The animal data-based identification and recognition system of claim 1 wherein the animal data is human data.

23. The animal data-based identification and recognition system of claim 1 wherein the at least one unique asset includes at least a portion of animal data.

24. The animal data-based identification and recognition system of claim 23 wherein the at least one unique asset includes at least a portion of non-animal data.

25. The animal data-based identification and recognition system of claim 1 wherein the at least one unique asset is one or more digital signatures, identifiers, patterns, trends, features, measurements, outliers, abnormalities, anomalies, characteristics, computed assets, insights, predictive indicators, or a combination thereof.

26. The animal data-based identification and recognition system of claim 1 wherein the at least one unique asset uses animal data derived from two or more source sensors to create, modify, or enhance the at least one unique asset.

27. The animal data-based identification and recognition system of claim 1 wherein the at least one unique asset uses two or more types of animal data to create, modify, or enhance the at least one unique asset.

28. The animal data-based identification and recognition system of claim 27 wherein the at least one unique asset includes at least a portion of non-animal data.

29. The animal data-based identification and recognition system of claim 27 wherein the at least one unique asset uses two or more types of animal data derived from the same source sensor to create, modify, or enhance the at least one unique asset.

30. The animal data-based identification and recognition system of claim 27 wherein the at least one unique asset uses two or more types of animal data derived from two or more source sensors to create, modify, or enhance the at least one unique asset.

31. The animal data-based identification and recognition system in claim 1 wherein the one or more computing devices dynamically create, modify, or enhance the at least one unique asset based upon the animal data collected by the one or more source sensors.

32. The animal data-based identification and recognition system in claim 1 wherein the one or more computing devices dynamically create, modify, or enhance the at least one unique asset based upon new reference animal data collected by the animal data-based identification and recognition system.

33. The animal data-based identification and recognition system of claim 1 wherein at least one of the one or more source sensors is a biosensor that gathers physiological, biometric, chemical, biomechanical, location, environmental, genetic, genomic, or other biological data from one or more targeted individuals.

34. The animal data-based identification and recognition system of claim 33 wherein the one or more source sensors include one or more biosensors that gather, or provide information that can be converted into, an animal data type selected from the group consisting of facial recognition data, eye tracking & recognition data, blood flow data, blood volume data, blood pressure data, biological fluid data, body composition data, biochemical data, pulse data, oxygenation data, core body temperature data, skin temperature data, galvanic skin response data, perspiration data, location data, positional data, audio data, biomechanical data, hydration data, heart-based data, neurological data, genetic data, genomic data, skeletal data, muscle data, respiratory data, kinesthetic data, ear acoustic authentication data, finger vein recognition data, fingerprint recognition data, footprint and foot dynamics data, hand geometry data, body odor recognition data, palm print recognition data, palm vein recognition data, skin reflection data, thermography recognition data, keystroke dynamics data, signature recognition data, speaker recognition data, voice recognition data, gait recognition data, lip motion data, or a combination thereof.

35. The animal data-based identification and recognition system of claim 34 wherein the at least one unique asset is derived from at least a portion of animal data gathered from the one or more biosensors.

36. The animal data-based identification and recognition system of claim 34 wherein the at least one unique asset is derived from two or more types of animal data gathered from the one or more biosensors.

37. The animal data-based identification and recognition system of claim 36 wherein the at least one unique asset is derived from animal data gathered from two or more biosensors.

38. The animal data-based identification and recognition system of claim 36 wherein the at least one unique asset includes at least a portion of non-animal data.

39. The animal data-based identification and recognition system of claim 36 wherein the at least one unique asset incorporates at least one of or any combination of: name, age, weight, height, eye color, skin color, hair color, birthdate, race, reference identification, country of origin, area of origin, ethnicity, current residence, addresses, phone number, gender, data quality assessment, information gathered from medication history, medical history, medical records, medical conditions, traits, health risks, inherited conditions, drug responses, DNA sequences, protein sequences and structures, drug/prescription records, allergies, family history, health history, blood analysis, physical shape, manually-inputted personal data, historical personal data, the one or more activities the targeted individual is engaged in while the animal data is collected, ambient temperature data related to the animal data, humidity data related to the animal data, barometric pressure data related to the animal data, elevation data related to the animal data, one or more associated groups, one or more nutritional habits, one or more activity habits, one or more health habits, one or more social habits, education records, criminal records, financial information, social data, employment history, marital history, relatives or kin history, relatives or kin medical history, relatives or kin health history, manually inputted personal data, historical personal data, or individual-generated data.

40. The animal data-based identification and recognition system in claim 33 wherein the biosensor is affixed to, are in contact with, or send one or more electronic communications in relation to or derived from, one or more targeted individuals including one or more of a targeted subject's body, eyeball, vital organ, muscle, hair, veins, biological fluid, blood vessels, tissue, or skeletal system, embedded in the one or more targeted individuals, lodged or implanted in one or more targeted individuals, ingested by the one or more targeted individuals, integrated to comprise at least a portion of the one or more targeted individuals, or integrated into or as part of, affixed to, or embedded within, a fabric, textile, cloth, material, fixture, object, or apparatus that contacts or is in communication with one or more targeted individuals, either directly or via one or more intermediaries.

41. The animal data-based identification and recognition system of claim 1 wherein at least one sensor of the one or more source sensors captures two or more types of animal data.

42. The animal data-based identification and recognition system of claim 1 wherein at least one sensor of the one or more source sensors is comprised of two or more sensors.

43. The animal data-based identification and recognition system of claim 1 wherein at least a portion of the animal data from an identified targeted subject or one or more derivatives thereof is distributed by the one or more computing devices to one or more other computing devices for consideration.

44. The animal data-based identification and recognition system of claim 1 wherein at least a portion of the animal data is distributed to one or more computing devices for consideration.

45. The animal data-based identification and recognition system of claim 44 wherein the animal data is distributed as part of an animal data consideration system.

46. The animal data-based identification and recognition system of claim 1 wherein the animal data includes metadata that incorporates one or more attributes related to targeted individual.

47. The animal data-based identification and recognition system of claim 1 wherein the reference animal data includes previously collected animal data.

48. The animal data-based identification and recognition system of claim 47 wherein at least a portion of previously collected animal data is derived from one or more sensors.

49. The animal data-based identification and recognition system of claim 47 wherein the reference animal data includes at least a portion of non-animal data.

50. The animal data-based identification and recognition system of claim 1 wherein the reference animal data includes animal data that is derived directly from the targeted individual, indirectly from the targeted individual, or a combination thereof.

51. The animal data-based identification and recognition system of claim 1 wherein the reference animal data includes data that is not derived directly or indirectly from the targeted individual but shares at least one attribute with the targeted individual, medical condition, or biological response.

52. The animal data-based identification and recognition system of claim 51 wherein the at least one attribute includes at least one of or any combination of: name, age, weight, height, eye color, hair color, skin color, birthdate, race, reference identification, country of origin, area of origin, ethnicity, current residence, addresses, phone number, gender, data quality assessment, information gathered from medication history, medical history, medical records, medical conditions, traits, health risks, inherited conditions, drug responses, DNA sequences, protein sequences and structures, drug/prescription records, allergies, family history, health history, blood analysis, physical shape, manually-inputted personal data, historical personal data, activities, ambient temperature data related to the animal data, humidity data related to the animal data, barometric pressure data related to the animal data, elevation data related to the animal data, one or more associated groups, one or more nutritional habits, one or more activity habits, one or more health habits, one or more social habits, education records, criminal records, financial information, social data, employment history, marital history, relatives or kin history, relatives or kin medical history, relatives or kin health history, manually inputted personal data, historical personal data, or individual-generated data.

53. The animal data-based identification and recognition system of claim 1 wherein creation, modification, or enhancement of the at least one unique asset occurs utilizing at least a portion of artificial data.

54. The animal data-based identification and recognition system of claim 53 wherein the artificial data is generated utilizing one or more artificial intelligence techniques.

55. The animal data-based identification and recognition system of claim 1 wherein creation, modification, or enhancement of the animal data or one or more derivatives thereof utilizes at least a portion of artificial data.

56. The animal data-based identification and recognition system of claim 55 wherein the artificial data is generated utilizing one or more artificial intelligence techniques.

57. The animal data-based identification and recognition system of claim 1 wherein the at least one unique asset or derivative of the animal data is created, modified, or enhanced utilizing one or more artificial intelligence techniques.

58. The animal data-based identification and recognition system of claim 57 wherein the one or more artificial intelligence techniques includes execution of one or more trained neural networks.

59. The animal data-based identification and recognition system of claim 58 wherein the one or more trained neural networks utilized to generate the at least one unique asset consists of one or more of the following types of neural networks: Feedforward, Perceptron, Deep Feedforward, Radial Basis Network, Gated Recurrent Unit, Autoencoder (AE), Variational AE, Denoising AE, Sparse AE, Markov Chain, Hopfield Network, Boltzmann Machine, Restricted BM, Deep Belief Network, Deep Convolutional Network, Deconvolutional Network, Deep Convolutional Inverse Graphics Network, Liquid State Machine, Extreme Learning Machine, Echo State Network, Deep Residual Network, Kohenen Network, Support Vector Machine, Neural Turing Machine, Group Method of Data Handling, Probabilistic, Time delay, Convolutional, Deep Stacking Network, General Regression Neural Network, Self-Organizing Map, Learning Vector Quantization, Simple Recurrent, Reservoir Computing, Echo State, Bi-Directional, Hierarchal, Stochastic, Genetic Scale, Modular, Committee of Machines, Associative, Physical, Instantaneously Trained, Spiking, Regulatory Feedback, Neocognitron, Compound Hierarchical-Deep Models, Deep Predictive Coding Network, Multilayer Kernel Machine, Dynamic, Cascading, Neuro-Fuzzy, Compositional Pattern-Producing, Memory Networks, One-shot Associative Memory, Hierarchical Temporal Memory, Holographic Associative Memory, Semantic Hashing, Pointer Networks, Encoder-Decoder Network, Recurrent Neural Network, Long Short-Term Memory Recurrent Neural Network, or Generative Adversarial Network.

60. The animal data-based identification and recognition system of claim 1 wherein gathered animal data from the one or more source sensors or one or more derivatives thereof are compared against the at least one unique asset by the one or more computing devices when executing one or more artificial intelligence techniques to identify the targeted subject, a medical condition, or a biological response.

61. The animal data-based identification and recognition system of claim 1 wherein the comparison between the at least one unique asset and gathered animal data or one or more derivatives thereof occurs once, intermittently, or regularly to verify the targeted individual, the targeted medical condition, or the targeted biological response.

62. The animal data-based identification and recognition system of claim 1 wherein the comparison between the at least one unique asset and gathered animal data or one or more derivatives thereof identifies multiple medical conditions or biological responses.

63. The animal data-based identification and recognition system of claim 1 wherein the comparison between the at least one unique asset and gathered animal data or one or more derivatives thereof identifies multiple targeted subjects.

64. The animal data-based identification and recognition system of claim 1 wherein the at least one unique asset is created, modified, or enhanced from two or more types of animal data that are captured across one or more time periods and one or more activities.

65. The animal data-based identification and recognition system of claim 64 wherein the at least one unique asset is created, modified, or enhanced using two or more types of animal data, collected across two or more time periods, collected when the targeted subject is engaged in one or more activities, or a combination thereof.

66. The animal data-based identification and recognition system of claim 1 wherein the at least one unique asset is created, modified, or enhanced using one or more artificial intelligence techniques that produce one or more biological representations of the targeted individual to understand one or more biological functions or processes of the targeted individual based upon their animal data to create, modify, or enhance the at least one unique asset.

67. The animal data-based identification and recognition system of claim 1 wherein a biological response is an activity, biological state, or medical event.

68. The animal data-based identification and recognition system of claim 1 wherein the one or more computing devices create, modify, or enhance the at least one unique asset from animal data that is both reference animal data and animal data gathered by the one or more source sensors from the targeted subject.

69. The animal data-based identification and recognition system of claim 1 wherein two or more unique assets are created that enable one or more targeted individuals, medical conditions, or biological responses to be identified in two or more ways.

70. The animal data-based identification and recognition system of claim 1 wherein once the animal data is verified and included as part of the reference animal data, one or more tags are created related to the targeted subject, medical condition, biological response, or a combination thereof.

71. The animal data-based identification and recognition system of claim 1 wherein the reference animal data is gathered from one or more other external sources.

72. The animal data-based identification and recognition system of claim 71 wherein the reference animal data is gathered from one or more computing devices and has attached metadata that enables the reference animal data to be associated with one or more subjects, medical conditions, biological responses, or a combination thereof.

73. An animal data-based identification and recognition system comprising:
one or more computing devices that gather reference animal data derived from, at least in part, one or more sensors wherein the one or more computing devices are operable to create, modify, or enhance at least one unique asset from the reference animal data for one or more known subjects that identify each of the one or more known subjects;
one or more source sensors that gather animal data from a targeted subject wherein the animal data is transmitted electronically;
a collecting computing device that is configured to (1) gather the animal data from the targeted subject via the one or more source sensors, (2) create, modify, or enhance at least one unique asset from at least a portion of the animal data derived from the targeted subject via the one or more source sensors for identifying the targeted subject as a known subject, and the collecting computing device is configured to either (i) gather the at least one created, modified, or enhanced unique asset derived from the reference animal data for the one or more known subjects, or (ii) provide the at least one unique asset derived from the targeted subject via the one or more source sensors, at least in part, to the one or more computing devices, wherein;
the collecting computing device or the one or more computing devices are configured to perform a comparison by comparing the at least one created, modified, or the enhanced unique asset from the one or more known subjects with the at least one created, modified, or the enhanced unique asset from the targeted subject; and
the comparison between two or more unique assets enables the collecting computing device or the one or more computing devices to identify the targeted subject as a known subject.

74. The animal data-based identification and recognition system of claim 73 wherein the one or more computing devices include the collecting computing device.

75. The animal data-based identification and recognition system of claim 73 wherein the collecting computing device is configured to the reference animal data.

76. The animal data-based identification and recognition system of claim 73 wherein at least a portion of the animal data from an identified targeted subject or its one or more derivatives is distributed by one or more computing devices to one or more other computing devices for consideration.

77. An animal data-based identification and recognition system comprising:
One or more computing devices that gather reference animal data derived from, at least in part, one or more sensors wherein the one or more computing devices are operable to create, modify, or enhance at least one unique asset for one or more known medical conditions or biological responses from the reference animal data that identify each of the one or more known medical conditions or biological responses;
one or more source sensors that gather animal data from a targeted subject wherein the animal data is transmitted electronically;
a collecting computing device configured to (1) gather the animal data from the targeted subject via the one or more source sensors, (2) create, modify, or enhance at least one unique asset from at least a portion of the animal data derived from the one or more source sensors for identifying one or more medical conditions or biological responses associated with the targeted subject, and the collecting computing device is further configured to either (i) gather the at least one created, modified, or enhanced unique asset derived from the reference animal data for the one or more known medical conditions or known biological responses, or (ii) provide the at least one unique asset derived from the targeted subject via the one or more source sensors, at least in part, to the one or more computing devices, wherein;
the collecting computing device or the one or more computing devices are configured to perform a comparison by comparing the at least one created, modified, or the enhanced unique asset for the one or more known medical conditions or biological responses with the at least one unique asset from the targeted subject; and
the comparison between two or more unique assets enables the collecting computing device or the one or more computing devices to identify one or more of the known medical conditions or biological responses associated with the targeted subject.

78. The animal data-based identification and recognition system of claim 77 wherein the one or more computing devices include the collecting computing device.

79. The animal data-based identification and recognition system of claim 77 wherein the collecting computing device is configured to source the reference animal data.

80. The animal data-based identification and recognition system of claim 77 wherein at least a portion of the animal data or its one or more derivatives from the identified one or more medical conditions or biological responses is distributed by one or more computing devices to one or more other computing devices for consideration.
